(19) United States
(12) Patent Application Publication
(10) Pub. No.: US 2025/0296976 A1
(43) Pub. Date: Sep. 25, 2025

(54) COMPOSITIONS AND METHODS COMPRISING ENGINEERED CHIMERIC ANTIGEN RECEPTOR AND MODULATOR OF CAR

(71) Applicant: AUTOLUS LIMITED, London (GB)

(72) Inventors: Martin Pulé, London (GB); Vijay Peddareddigari, London (GB); Christian Itin, London (GB)

(21) Appl. No.: 19/073,904

(22) Filed: Mar. 7, 2025

Related U.S. Application Data
(62) Division of application No. 17/436,673, filed on Sep. 7, 2021.

(30) Foreign Application Priority Data
Mar. 8, 2019 (GB) 1903237.4
Oct. 2, 2019 (GB) 1914216.5
Nov. 5, 2019 (GB) 1916077.9

(51) Int. Cl. C07K 14/725; A61K 40/11; A61K 40/31; A61K 40/42; A61K 48/00; C07K 14/71; C07K 14/715; C12N 15/86

(52) U.S. Cl. CPC C07K 14/7051; A61K 40/11; A61K 40/31; A61K 40/4211; A61K 40/4212; A61K 40/4258; A61K 40/4276; A61K 48/005; C07K 14/71; C07K 14/715; C12N 15/86; A61K 2239/11; A61K 2239/31; A61K 2239/38; A61K 2239/47; C07K 2319/03; C12N 2740/16043; C12N 2800/40

(57) ABSTRACT

There is provided method for making a cell composition which comprises step of transducing a population of cells with a mixture of at least two viral vectors, wherein at least one vector comprises a nucleic acid sequence which encodes a chimeric antigen receptor (CAR); and wherein at least one vector comprises a nucleic acid encoding an activity modulator which modulates the activity of the CAR, of a cell expressing the CAR, or of a target cell. There is also provided a cell composition made by such a method and its use in the treatment of diseases such as cancer.

### CROSS-REFERENCE TO RELATED APPLICATIONS

This is a Divisional of U.S. application Ser. No. 17/436,673, filed Sep. 7, 2021, which is a U.S. National Phase of International Application No. PCT/GB2020/050535, filed Mar. 6, 2020, which claims priority to Great Britain Application No. 1903237.4, filed Mar. 8, 2019, Great Britain Application No. 1914216.5, filed Oct. 2, 2019 and Great Britain Application No. 1916077.9 filed Nov. 5, 2019.

### INCORPORATION BY REFERENCE OF MATERIAL SUBMITTED ELECTRONICALLY

A Sequence Listing is incorporated herein by reference as part of the disclosure. The sequence listing was submitted as a text file named “56191B_SubSeqlisting.xml”, which was created on Apr. 11, 2025 and is 96,462 bytes in size.

### FIELD OF THE INVENTION

The present invention relates viral vector compositions, their use to transduce cells and cell compositions made by such methods.

### BACKGROUND TO THE INVENTION

Tumour heterogeneity describes the observation that different tumour cells can show distinct morphological and phenotypic profiles, including cellular morphology, gene expression, metabolism, motility, proliferation, and metastatic potential.

Heterogeneity occurs between patients, between tumours (inter-tumour heterogeneity) and within tumours (intra-tumour heterogeneity). Multiple types of heterogeneity have been observed between tumour cells, stemming from both genetic and non-genetic variability.

Heterogeneity between tumour cells can be further increased due to heterogeneity in the tumour microenvironment. Regional differences in the tumour (e.g. availability of oxygen) impose different selective pressures on tumour cells, leading to a wider spectrum of dominant subclones in different spatial regions of the tumour. The influence of microenvironment on clonal dominance is also a likely reason for the heterogeneity between primary and metastatic tumours seen in many patients, as well as the inter-tumour heterogeneity observed between patients with the same tumour type.

The heterogeneity of cancer cells introduces significant challenges in designing effective treatment strategies.

For example, heterogenic tumours may exhibit different sensitivities to cytotoxic drugs among different clonal populations. This is attributed to clonal interactions that may inhibit or alter therapeutic efficacy.

Drug administration in heterogenic tumours will seldom kill all tumour cells. The initial heterogenic tumour population may bottleneck, such that few drug resistant cells (if any) will survive. This allows resistant tumour populations to replicate and grow a new tumour through the branching evolution mechanism (see above). The resulting repopulated tumour is heterogenic and resistant to the initial drug therapy used. The repopulated tumour may also return in a more aggressive manner.

### Chimeric Antigen Receptors (CARs)

Chimeric antigen receptors are proteins which graft the specificity of, for example, a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals (see FIG. 1A).

The most common form of these molecules are fusions of single-chain variable fragments (scFv) derived from monoclonal antibodies which recognize a target antigen, fused via a spacer and a trans-membrane domain to a signalling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

Successful CAR treatment depends on expression of the target antigen by tumour cells. In heterogenic tumours, in particular solid cancers, antigen expression is heterogeneous, and it may not be possible to find a single target antigen expressed by all cancer cells.

Moreover, emerging data from CAR T-cell trials in B-cell malignancies demonstrate that a common mechanism of resistance to this class of therapeutics is the emergence of tumours with loss or downregulation of the target antigen. Antigen loss or antigen-low escape is likely to emerge as an even greater barrier to success in solid tumours, which manifest greater heterogeneity in target antigen expression. Potential approaches to overcome this challenge include engineering CAR T cells to achieve multi-specificity and to respond to lower levels of target antigen and more efficient induction of natural antitumor immune responses as a result of CAR-induced inflammation.

Clinical studies of CAR T-cells have established that CAR T-cell engraftment, expansion and persistence are a pre-requisite for clinical activity, particularly sustained responses. A key reason for poor persistence of CAR-T cells in vivo, particularly CAR-T cells for the treatment of solid cancers, is that the cells struggle to overcome the hostile microenvironment of the tumour. In particular, CAR T-cells may fail to engraft and expand within a solid cancer tumour bed.

CAR T-cell persistence and activity can be enhanced by administration of cytokines, or by engineering the CAR T-cell to secrete or express cytokine, toxins or other factors. However, these approaches have limitations: systemic administration of cytokines can be toxic; constitutive production of cytokines may lead to uncontrolled proliferation and transformation.

There is thus a need for alternative CAR treatment approaches which address the problems commonly encountered with CAR-T cell therapy, particularly bearing in mind the heterogeneity between patients, and between tumour cells and tumour cell sites within the same patient.

### DESCRIPTION OF THE FIGURES

FIG. 1—Schematic diagram showing a classical chimeric antigen receptors (a) Basic schema of a chimeric antigen receptor; (b) First generation receptors; (c) Second generation receptors; (d) Third generation receptors.

FIG. 2—Schematic diagram illustrating the mechanism of a) T-cell activation and b) T-cell inhibition in vivo

FIG. 3—Schematic diagram illustrating the JAK-STAT signaling pathway (activated by α-interferon).

FIG. 4—Different binding domain formats of chimeric antigen receptors (a) Fab CAR format; (b) dAb CAR format; (c) scFv CAR format

FIG. 5—Schematic diagram illustrating the difference in transduced cell compositions obtained by transduction with a single vector co-expressing two genes (A); and a mixture of two vectors, each expressing a single gene (B). When cells are transduced with a single vector having a bicistronic cassette, every cell which is successfully transduced will express both transgenes at a stoichiometry of approximately 1:1 (A). However, when cells are transduced with multiple vectors, a much more heterogeneous population is obtained: transduced cells may express the first transgene alone, the second transgene alone or both transgenes. In cells expressing both transgenes the relative level of expression of gene A and gene B is completely variable (B).

FIG. 6—Scatter plot showing that when T cells are transduced with a mixture of vectors (A and B), a proportion of cells are left untransduced; a proportion of cells are transduced with vector A alone; a proportion of cells are transduced with vector B alone; and a proportion of cells are transduced with vectors A and B. In this study, cells are transduced with a mixture of vectors, one of which expresses an anti-CD19 CAR and one of which expresses a CD22 CAR.

FIG. 7—Schematic diagram illustrating the molecules expressed by the vectors used in the dual vector composition described in Example 3. Vector 1 expresses a CAR with an antigen-binding domain which binds GD2 (GD2 CAR), a constitutively active cytokine receptor (CCR) and a sort/suicide gene (RQR8). Vector 2 expresses the same GD2 CAR, a dominant negative SHP-2 (ΔSHP2); a dominant negative transforming growth factor (TGF)βII receptor (ΔTGFbRII) and the same sort/suicide gene (RQR8).

FIG. 8—Investigating the capacity of single and dual transduced T cell populations to kill GD2-expressing (SupT1 GD2) and non-expressing (SupT1 NT) target cells.

FIG. 9—Investigating the proliferation of single and dual transduced T cell populations following culture in cytokine-free complete cell culture media for 7 days without further antigen stimulus.

FIG. 10—Investigating the capacity of single and dual transduced T cell populations to kill GD2-expressing (SupT1 GD2) and non-expressing (SupT1 NT) target cells in the presence or absence of TGFβ.

FIG. 11—Investigating cytokine production (IFNγ) from single and dual transduced T cell populations following co-culture with GD2-expressing (SupT1 GD2) and non-expressing (SupT1 NT) target cells in the presence or absence of TGFβ.

FIGS. 12A-12D—Results of an in vivo assay investigating the anti-tumour activity of T cells transduced with the dual vector composition by intravenous administration in an established neuroblastoma xenograft model in NSG mice. 1×10⁶ CHLA-255 Fluc cells were injected i.v. into female NSG mice. Xenografts were left to establish for 15 days until stable engraftment was detectable by BLI. CAR-T cells were made either by transducing cells with a single vector expressing a GD2 CAR (GD2 CAR) or by transducing cells with the dual vector composition described in Example 3 and Illustrated in FIG. 7 (GD2 CAR+IL7 CCR/GD2 CAR+dSHP2+dTGFbRII). CAR T-cells were administered i.v. at a dose of 3×10⁶ CAR T-cells/mouse. Quantitated bioluminescent signal of CHLA-255 Fluc was plotted over time as total flux (photons/s) FIG. 12A. Graph showing fluorescent signal over time for mice receiving CAR-T cells expressing GD2 CAR alone (GD2 CAR); untransduced T cells (NT) or buffer alone (PBS). FIG. 12B Ventral images of mice obtained on days −1, 2, 7, 10 and 14 following administration of CAR-T cells expressing GD2 CAR alone (GD2 CAR); untransduced T cells (NT) or buffer alone (PBS) FIG. 12C Graph showing fluorescent signal over time for mice receiving cells transduced with the dual vector composition described in Example 3 and Illustrated in FIG. 7 (GD2 CAR+IL7 CCR/GD2 CAR+dSHP2+dTGFbRII); untransduced T cells (NT) or buffer alone (PBS). FIG. 12D Ventral images of mice obtained on days −1, 2, 7, 10 and 14 following administration of cells transduced with the dual vector composition described in Example 3 and Illustrated in FIG. 7 (GD2 CAR+IL7 CCR/GD2 CAR+dSHP2+dTGFbRII); untransduced T cells (NT) or buffer alone (PBS).

FIGS. 13A-13B—FIG. 13A Schematic diagram illustrating the molecules expressed by the vectors used in the triple vector composition “AUTO7” described in Example 6.
Vector A expresses truncated SHP2 (dSHP2); the safety switch RQR8; an anti-PSMA CAR based on a novel humanized binder 7A12 (7A12-28z); and dominant negative TGFβRII (dnTβRII).
Vector B expresses a constitutively active IL-7 receptor (CCR_IL7).
Vector C expresses the RapaCasp9 suicide gene (RapaCasp9); CD19 (dCD19) and an IL-12 module (flexiIL-12). “2A” is an co-expression sequence based on the FMDV 2A peptide.

FIG. 13B An alternative arrangement for the triple vector composition. Components are as indicated for FIG. 13A above. dNGFR is truncated Nerve Growth Factor Receptor which is used as a marker protein.

FIG. 14—Results of a FACS-based killing (FBK) assay investigating the capacity of single, double and triple transduced T cells to kill PSMA-expressing target cells. A: FBK after 24 h of incubation using cytofluorimetry analysis to show survival of target cells. B: Secretion of IL-2 and IFNγ by CAR T-cells measured by collecting supernatant at 24 hr from the co-cultures described and detecting by ELISA.
AUTO7 was investigated as product of a single transduction using the vector A. (“AUTO7/A”), or double transduction using vectors A and B (“AUTO7/AB”), or triple transduction using vectors A, B and C (“AUTO7/ABC”). AUTO7 was tested against a second generation CAR developed using the same anti PSMA binder 7A12 (“Parental”)
SupT1 target cells were engineered to express human PSMA antigen at different levels (SupT1-PSMAhigh, SupT1-PSMAlow) were used as target cells. Non-engineered SupT1 cells (SupT1-NT) were used as a negative control. CAR T-cells were co-cultured with target cells at 1:2 effector to target ratios.

FIGS. 15A-15B—Results of a FACS-based killing (FBK) assay investigating the capacity of single, double and triple transduced T cells to kill PSMA-expressing target cells following culture in cytokine-free complete cell culture media. After 7 days culture in “starvation conditions” CAR T-cells were co-cultured with SupT1-PSMAhigh and SupT1-PSMAlow targets cells (or SupT1-NT cells as negative control). at 1:2 and 1:8 effector to target ratios. FIG. 15A: FBK after 24 h of incubation using cytofluorimetry analysis to show survival of target cells. FIG. 15B: Secretion of IL-2 and IFNγ by CAR T-cells measured by collecting supernatant at 24 hr from the co-cultures described and detecting by ELISA.

FIG. 16—Results of a FACS-based killing (FBK) assay investigating the capacity of single, double and triple transduced T cells to kill PSMA-expressing target cells in the presence or absence of TGFβ. CAR T-cells were co-cultured with SupT1-PSMAhigh and SupT1-PSMAlow targets for 7 days at ratio 1:2 and 1:8 (E:T) either in the presence or absence of 10 ng/ml TGFβ1 (SupT1-NT were used as control). Target cell killing was quantified by FACS and normalised to targets alone.

FIG. 17—Results of a FACS-based killing (FBK) assay investigating the capacity of single, double and triple transduced T cells to kill PSMA-expressing target cells following repeated restimulation with target cells. Transduced T-cells were co-cultured with SupT1-PSMAhigh or SupT1-PSMAlow target cells at 1:1 ratio (E:T). Every 7 days CAR T-cells were re-stimulated with 5×10⁴ SupT1 cells. Target cell killing was quantified by FACS before each new re-stimulation.

FIGS. 18A-18B—In vivo assay investigating the anti-tumour activity of T cells transduced with the triple vector composition by intravenous administration in a prostate cancer xenograft model in NSG mice. 5×10⁶ PSMA positive PC3 human cell lines were injected in the flank of female NSG mice. Xenografts were left to establish for 3 weeks until stable engraftment was detectable by palpation and calliper measurement. CAR T-cells were administered i.v. at a dose of 1×10⁶ CAR T-cells/mouse. Calliper measurement was taken 2/3 times a week. FIG. 18A: Data for mice receiving cells made by single transduction using the vector A. (“AUTO7/A”), double transduction using vectors A and B (“AUTO7/AB”), triple transduction using vectors A, B and C (“AUTO7/ABC”); or a second generation CAR developed using the same anti PSMA binder 7A12 (“Parental”); FIG. 18B: Summary of data shown in FIG. 18A.

### SUMMARY OF ASPECTS OF THE INVENTION

The present inventors have developed a combinatorial approach to address the issue of tumour cell and microenvironment heterogeneity to CAR therapies.

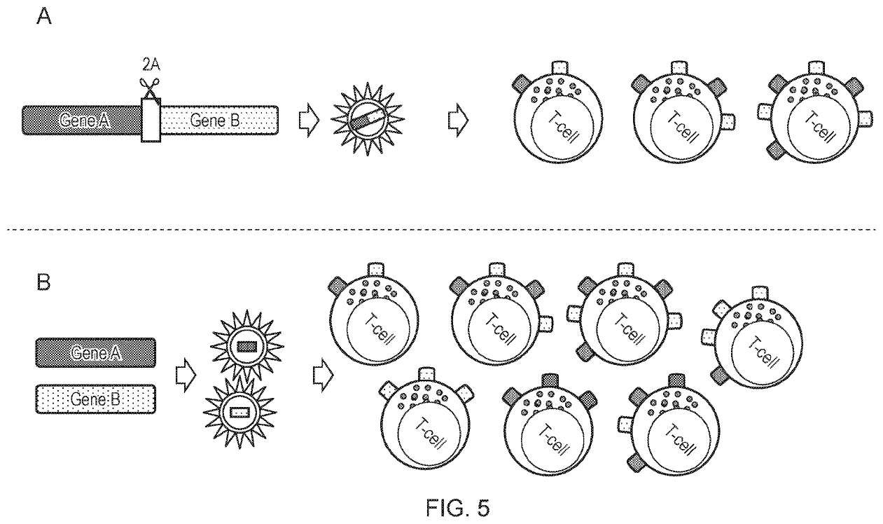

When cells are transduced with multiple vectors simultaneously, the resulting product will be a mixture of cells which are singly and combinatorially transduced. For example, if cells are transduced with two vectors, one comprising transgene A and one comprising transgene B, the transduced cells will be a mixture of cells expressing A alone; B alone; and cell expressing both A and B (FIG. 5B). For cells transduced with three vectors each comprising a transgene, the resulting transduced cells will be a mixture of: A alone; B alone; C alone; A and B; A and C; B and C; and cells expressing A, B and C.

The present invention involves using such a mixture as a therapeutic CAR-T-cell product. The use of a combinatorial product gives in-built flexibility which enhances the product's capacity to adapt to differences in target cells and in tumour microenvironment.

For example, the vectors may encode a combination of different CARs, which may vary in e.g. their antigen binding domains and/or costimulatory domain(s). Alternatively or in addition, one or more of the vectors may encode an activity modulator which modulates the activity of the CAR, of a cell expressing the CAR, or of a target cell. When the combinatorial CAR T-cell composition is administered in vivo, the cells will migrate to different tumour sites within the body. Whichever sub-population of CAR-T cells expressing a particular combination of CAR(s) and activity modulator(s) is best equipped to survive, persist and kill target cells at that location will have a selective advantage over the other sub-populations in the product and will win out. In this way the CAR-T cell product can adapt to tumour heterogeneity between patients and between sites in the same patient.

The method can also be used to establish which combination of vectors is optimal for generating CAR-T cells for the treatment a particular disease or disease subtype by analysing a patient to see which subpopulation of CAR-T cells in the patient shows the best persistence and/or activity.

Thus, in a first aspect, the present invention provides a method for making a cell composition which comprises step of transducing a population of cells with a mixture of at least two viral vectors, wherein at least one vector comprises a nucleic acid sequence which encodes a chimeric antigen receptor (CAR).

The method of the invention may equally be applied to cells expressing engineered T-cell receptors. Any and all of the aspects and embodiments described below are also applicable to engineered TCR-expressing cells.

The mixture may comprise two, three, four, five or more viral vectors.

Two or more viral vectors in the mixture may each comprise a CAR-encoding nucleic acid sequence. The first CAR and second CAR may have different antigen-binding domains and/or different spacers and/or different endodomains.

The CAR encoding nucleic acid of one or more viral vector(s) may encode two or more CARs. For example, the nucleic acid may encode a CAR logic gate, such as an OR gate.

The present invention provides method for making a cell composition which comprises step of transducing a population of cells with a mixture of at least two viral vectors, wherein at least one vector comprises a nucleic acid sequence which encodes a chimeric antigen receptor (CAR); and wherein at least one vector comprises a nucleic acid encoding an activity modulator which modulates the activity of the CAR, of a cell expressing the CAR, or of a target cell.

The technology of the invention, insofar as it relates to the expression of activity modulator(s) applies equally to cells for adoptive cell therapy which do not express as CAR or engineered TCR, such as tumour infiltrating lymphocytes (TILs). Any and all of the aspects and embodiments described below insofar as they relate to the expression of activity modulator(s), are also applicable generally to therapeutic T cells such as TILs.

One or more viral vectors in the mixture may comprise a nucleic acid sequence encoding both a CAR and an activity modulator, so that a cell transduced with this vector co-expresses the CAR and the activity modulator.

An activity modulator which modulates the activity of the CAR may affect the balance between phosphorylation and dephosphorylation at the CAR-expressing cell:target cell synapse. For example, the activity modulator may comprise a kinase domain capable of phosphorylating Immunoreceptor tyrosine-based activation motifs (ITAMs) in the CAR endodomain.

Alternatively the activity modulator may be capable of recruiting a kinase to be brought into proximity with the CAR, where it can phosphorylate ITAMs in the CAR endodomain.

An activity modulator which modulates the activity of CAR-expressing cell may be an intracellular molecule or may be expressed at the cell surface.

In vivo, membrane-bound immunoinhibitory receptors such as CTLA4, PD-1, LAG-3, 2B4 or BTLA 1 inhibit T cell activation. The activity modulator may block or affect this inhibitory pathway.

The activity modulator may be an agent, such as an antibody, which binds to an inhibitory immunoreceptor or binds to a ligand for an inhibitory immunoreceptor.

An activity modulator which blocks or reduces the inhibition mediated by inhibitory immunoreceptors such as CTLA4, PD-1, LAG-3, 2B4 or BTLA 1 may tip the balance of phosphorylation:dephosphorylation at the T-cell:target cell synapse in favour of phosphorylation of ITAMs, leading to T-cell activation. For example, the activity modulator may block or reduce the phosphorylation of ITIMs in the endodomain of inhibitory receptor(s) or may block or reduce the dephosphorylation of ITAMs in the CAR signalling domain by proteins such as SHP-1 and SHP-2.

The activity modulator may be a dominant negative SHP-1 or SHP-2.

For example, the activity modulator may be a truncated protein which comprises an SH2 domain from a protein which binds a phosphorylated immunoreceptor tyrosine-based inhibition motif (ITIM), such as SHP-1 or SHP-2, but lacks a phosphatase domain.

The activity modulator may be a cytokine or chemokine such as IL12, flexiIL-12, GM-CSF, IL7, IL15, IL21, IL2 or CCL19.

Alternatively the activity modulator may have an effect of a cytokine/chemokine signalling pathway in the CAR-expressing cell.

For example, the activity modulator may be a chimeric cytokine receptor which comprises a cytokine receptor endodomain. The exodomain may be derived from a different cytokine-receptor, or may not be from a cytokine receptor. The exodomain may bind a ligand, for example a tumour antigen or secreted factor. Presence of the ligand may cause two chains of a cytokine receptor endodomain to associate, leading to cytokine signalling.

The activity modulator may be a constitutively active chimeric cytokine receptor. The activity modulator may comprise two chains which dimerise, either spontaneously or in the presence of an agent (a chemical inducer of dimerization or CID) bringing together two cytokine receptor endodomains.

The activity modulator may affect the JAK/STAT cytokine signalling pathway. The activity modulator may comprise an inducible or constitutively active Signal Transducer and Activator of Transcription (STAT) or Janus kinase (JAK).

The activity modulator may be or comprise an adhesion molecule or a transcription factor. The transcription factor may prevent or reduce differentiation and/or exhaustion of the CAR-expressing cell.

The activity modulator of the present invention may modulate TGFβ signalling.

For example, the activity modulator may block or reduce TGFβ binding to TGFβ receptor; it may compete with TGFβ or TGFβR for binding to TGFβR or TGFβ; alternatively it may modulate the downstream TGFβ signalling for example via SMADs. The activity modulator may be a dominant negative TGFβ receptor.

The activity modulator of the present invention may provide co-stimulatory signal to the T-cell.

For example, the activity modulatory may be a TNF receptor, a chimeric TNF receptor or a TNF receptor ligand.

The activity modulator may modulate the activity of the target cell, for example, a tumour cell.

The agent may be a toxin, a pro-drug or a pro-drug activating compound.

The activity modulator may be an enzyme which is capable of synthesising a small molecule when expressed or expressed in combination in a cell. The expression of such an enzyme or combination of enzymes in a CAR-expressing cell can confer on that cell the capacity to synthesise a small molecule, such as a small molecule which is toxic to a tumour cell.

Alternatively, the activity modulator may be an enzyme which is secreted by the CAR-expressing cell. The activity modulator be one or more enzymes which, when secreted or expressed at the cell surface, causes depletion of a molecule extracellular to the engineered cell which is:
(i) required by a tumour cell for survival, proliferation, metastasis or chemoresistance, and/or
(ii) detrimental to the survival, proliferation or activity of the engineered cell.

The enzyme(s) may cause the depletion of for example an amino acid or amino acid metabolite, a nucleobase (such as a nucleoside or nucleotide) or a lipid.

In the method of the invention, the mixture of viral vectors may comprise at least one vector which comprises a nucleic acid sequence which encodes a dominant negative SHP-1 or SHP-2; and at least one vector which comprises a nucleic acid sequence which encodes a dominant negative transforming growth factor (TGF)β receptor.

The mixture of viral vectors may comprise two, three, four, five or six viral vectors, at least one of which comprises a nucleic acid sequence encoding a CAR; and at least one of which comprises a nucleic acid sequence encoding an activity modulator.

The method may involve the following steps:
(i) transducing a population of cells with a mixture of at least two viral vectors; and
(ii) selecting CAR-expressing cells from the transduced cell population from step (i).

Alternatively, where each of the viral vectors in the mixture comprises a nucleic acid sequence encoding a CAR, it may not be necessary to select or purify CAR-expressing cells from the transduced cell population.

In a second aspect, the present invention provides a viral vector composition. The viral vector composition may comprise a mixture of two of more vectors. The vector composition may be suitable for use in the method of the first aspect of the invention.

The viral vector composition may comprise a first vector and a second vector, both of which comprise a nucleic acid sequence encoding a chimeric antigen receptor (CAR).

The CAR expressed by the first vector may be the same as the CAR expressed by the second vector. For example, the CAR expressed by the first vector may have the same antigen-binding domain as the CAR expressed by the second vector.

The first vector and/or second vector may also express an activity modulator which modulates the activity of the CAR, of a cell expressing the CAR, or of a target cell. Where the first vector and second vector both express an activity modulator, the first vector and second vector may express a different activity modulator or a different combination of activity modulators.

For example, the first vector and second vector may express one or more activity modulator(s) selected from: a dominant negative SHP-1 or SHP-2; a dominant negative transforming growth factor (TGF)β receptor; and a constitutively active chimeric cytokine receptor.

In one arrangement, the first vector may comprise a nucleic acid sequence encoding a dominant negative SHP-1 or SHP-2 and a nucleic acid sequence encoding a dominant negative transforming growth factor (TGF)β receptor; and the second vector may comprise a nucleic acid sequence encoding a constitutively active chimeric cytokine receptor.

Where the first and second vectors encode the same CAR, the CAR may have an antigen-binding domain which binds disialoganglioside (GD2).

The first vector and/or the second vector may comprise a nucleic acid sequence encoding a suicide gene.

The present invention also provides a cell composition made by a method according of the invention or made by transducing a cell with a vector composition of the invention ex vivo.

In a third aspect, the present invention provides a cell composition made by a method according to the first aspect of the invention or made by transducing a population of cells with a viral vector composition of the second aspect of the invention.

In a fourth aspect, there is provided a method for treating a disease in a subject which comprises the step of administering a cell composition according to the third aspect of the invention to the subject.

In a fifth aspect there is provided a cell composition according to the third aspect of the invention for use in treating and/or preventing a disease.

In a sixth aspect, there is provided the use of a cell composition according to third aspect of the invention in the manufacture of a medicament for treating and/or preventing a disease.

In a seventh aspect, there is provided a method for determining the optimal combination of components for a CAR-expressing cell to treat a disease, which comprises the following steps:
(i) administering a cell composition according to the second aspect of the invention to a subject having the disease;
(ii) monitoring the patient or samples from the patient to determine which sub-population of cells in the cell composition show the greatest level of engraftment and/or proliferation; and
(iii) analysing the phenotype/genotype of the cells in the sub-population to ascertain the CAR(s) and/or activity modulator(s) expressed by those cells.

### Further Aspects

The present invention also provides additional aspects which are summarised in the following numbered paragraphs:
1. A nucleic acid construct which comprises a nucleic acid sequence encoding a dominant negative SHP-2 and a nucleic acid sequence encoding a dominant negative TGFβ receptor.
2. A nucleic acid construct according to paragraph 1, which has the structure:
dnSHP-coexpr-dnTGFβR, or
dnTGFβR-coexpr-dnSHP
   in which:
dnSHP is a nucleic acid sequence encoding dominant negative SHP-2
“coexpr” is a nucleic acid sequences enabling coexpression of the two polypeptides as separate entities
“dnTGFβR” is a nucleic acid sequence encoding a dominant negative TGFβ receptor.
3. A nucleic acid construct according to paragraph 1 which also comprises a nucleic acid sequence encoding a CAR.
4. A nucleic acid construct according to paragraph 3, which has the structure:
CAR-coexpr1-dnSHP-coexpr2-dnTGFβR
CAR-coexpr1-dnTGFβR-coexpr2-dnSHP
dnTGFβR-coexpr1-CAR-coexpr2-dnSHP
dnTGFβR-coexpr1-dnSHP-coexpr2-CAR
dnSHP-coexpr1-dnTGFβR-coexpr2-CAR or
dnSHP-coexpr1-CAR-coexpr2-dnTGFβR
   in which:
dnSHP is a nucleic acid sequence encoding dominant negative SHP-2
“coexpr1” and “coexpr2” which may be the same or different, are nucleic acid sequences enabling coexpression of the three polypeptides as separate entities
“dnTGFβR” is a nucleic acid sequence encoding a dominant negative TGFβ receptor; and
“CAR” is a nucleic acid sequence encoding a chimeric antigen receptor.
5. A nucleic acid construct according to paragraph 4, which has the structure: dnSHP-coexpr1-CAR-coexpr2-dnTGFβR
6. A nucleic acid construct according to any of paragraphs 3 to 5, wherein the CAR binds one of the following target antigens: CD19, CD22, BCMA, PSMA, CD79, GD2 or FCRL5.
7. A nucleic acid construct according to paragraph 3, which comprises a bicistronic nucleic acid sequence encoding two CARs.
8. A nucleic acid construct according to paragraph 7, which has the structure:
dnSHP-coexpr1-CAR1-coexpr2-CAR2-coexpr3-dnTGFβR
   in which:
“dnSHP” is a nucleic acid sequence encoding dominant negative SHP-2
“coexpr1”, “coexpr2” and “coexpr3” which may be the same or different, are nucleic acid sequences enabling coexpression of the four polypeptides as separate entities;
“CAR1” is a nucleic acid sequence encoding a first chimeric antigen receptor;
“CAR2” is a nucleic acid sequence encoding a second chimeric antigen receptor; and
“dnTGFβR” is a nucleic acid sequence encoding a dominant negative TGFβ receptor.
9. A nucleic acid construct according to paragraph 7 or 8, wherein one CAR binds CD19 and the other CAR binds CD22.
10. A nucleic acid construct according to paragraph 6, wherein the CAR binds CD19 and has an antigen-binding domain which comprises
   a) a heavy chain variable region (VH) having complementarity determining regions (CDRs) with the following sequences:
   CDR1—GYAFSSS (SEQ ID No. 1);
   CDR2—YPGDED (SEQ ID No. 2)
   CDR3—SLLYGDYLDY (SEQ ID No. 3); and
   b) a light chain variable region (VL) having CDRs with the following sequences:
   CDR1—SASSSVSYMH (SEQ ID No. 4);
   CDR2—DTSKLAS (SEQ ID No. 5)
   CDR3—QQWNINPLT (SEQ ID No. 6).
11. A nucleic acid construct according to paragraph 10, wherein the antigen binding domain comprises a VH domain having the sequence shown as SEQ ID No. 7 and a VL domain having the sequence shown as SEQ ID No 8.
12. A nucleic acid construct according to any preceding paragraph which also comprises a nucleic acid sequence encoding a suicide gene.
13. A vector comprising a nucleic acid construct according to any preceding paragraph.
14. A kit of vectors comprising a first vector according to paragraph 13 and a second vector comprising a nucleic acid sequence which encodes a chimeric antigen receptor (CAR) or activity modulator.
15. A kit of vectors comprising a first vector which comprises a nucleic acid sequence encoding a dominant negative SHP-2 and a second vector which comprises a nucleic acid sequence encoding a dominant negative TGFβ receptor.
16. A kit of vectors according to paragraph 15, in which the first vector comprises a nucleic acid sequence encoding a first CAR and the second vector comprises a nucleic acid sequence encoding a second CAR.
17. A kit of vectors according to paragraph 16, wherein the first and second CARs have the same target antigen.
18. A kit of vectors according to paragraph 16, wherein the first and second CARs are the same.
19. A kit of vectors comprising a first vector according to paragraph 12, and a second vector encoding a chimeric cytokine receptor (CCR).
20. A kit of vectors according to paragraph 19, in which the first vector comprises a nucleic acid sequence encoding a first CAR and the second vector comprises a nucleic acid sequence encoding a second CAR.
21. A kit of vectors according to paragraph 20, wherein the first and second CARs have the same target antigen.
22. A kit of vectors according to paragraph 20, wherein the first and second CARs are the same.
23. A kit of vectors according to any of paragraphs 14 to 22, in which the first vector comprises a nucleic acid sequence encoding a first suicide gene and the second vector comprises a nucleic acid sequence encoding a second suicide gene.
24. A kit of vectors according to paragraph 23, wherein the first and second suicide genes are triggered by the same molecule.
25. A kit of vectors according to paragraph 23, wherein the first and second suicide genes are triggered by the different molecules.
26. A kit of vectors according to any of paragraphs 23 to 25, in which the vectors have the following structure:
Vector 1: CAR1-coexpr1-SG1-coepr2-dSHP2-coexpr3-dnTGFβR
Vector 2: CAR2-coexpr4-SG2-coepr5-CCR
   in which:
“CAR1” is a nucleic acid sequence encoding a first chimeric antigen receptor;
“coexpr1”, “coexpr2”, “coexpr3”, “coexpr4”, “co-expr5” which may be the same or different, are nucleic acid sequences enabling coexpression of the seven polypeptides as separate entities;
“SG1” is a nucleic acid sequence encoding a first suicide gene;
“dnSHP” is a nucleic acid sequence encoding dominant negative SHP-2;
“dnTGFβR” is nucleic acid sequence encoding a dominant negative TGFβ receptor;
“CAR2” is a nucleic acid sequence encoding a second chimeric antigen receptor which may or may not be the same as CAR1;
“SG2” is a nucleic acid sequence encoding a first suicide gene which may or may not be the same as SG1; and
“CCR” is a nucleic acid sequence encoding a chimeric cytokine receptor.
27. A kit of vectors according to any of paragraphs 14 to 26, which also comprises a third vector comprising a nucleic acid sequence encoding a cytokine.
28. A kit of vectors according to paragraph 27, wherein the cytokine is IL-12 or Flexi-IL12
29. A kit of vectors according to paragraph 28, in which the vectors have the following structure:
Vector 1: dnSHP2-coexpr1-SG1-coepr2-CAR-coexpr3-dnTGFβR
Vector 2: CCR
Vector 3: SG2-coexpr4-flexiIL 12
   in which:
“dnSHP” is a nucleic acid sequence encoding dominant negative SHP-2;
“coexpr1”, “coexpr2”, “coexpr3” and “coexpr4”, which may be the same or different, are nucleic acid sequences enabling coexpression of the six polypeptides on Vectors 1 and 3 as separate entities;
“SG1” is a nucleic acid sequence encoding a first suicide gene;
“CAR” is a nucleic acid sequence encoding a chimeric antigen receptor;
“dnTGFβR” is nucleic acid sequence encoding a dominant negative TGFβ receptor;
“CCR” is a nucleic acid sequence encoding a chimeric cytokine receptor;
“SG2” is a nucleic acid sequence encoding a first suicide gene which may or may not be the same as SG1; and
“flexiIL12” is a nucleic acid sequence encoding flexi-IL-12.
30. A vector composition comprising a mixture of: a vector according to paragraph 13 and at least one other viral vector; first and second vectors as defined in any of paragraphs 14 to 26; or first, second and third vectors as defined in any of paragraphs 27 to 29.
31. A method for making a cell composition which comprises step of transducing a population of cells with a vector according to paragraph 13, a kit of vectors according to any of paragraphs 14 to 29, or a vector composition according to paragraph 30.
32. A cell which co-expresses dominant negative SHP-2 and dominant negative TGFβ receptor.
33. A cell according to paragraph 32, which also expresses one or more chimeric antigen receptor(s) (CAR(s)).
34. A cell according to paragraph 33, wherein the CAR(s) is/are as defined in any of paragraphs 6 to 11.
35. A cell composition made by a method according to paragraph 31 or comprising a plurality of cells according to any of paragraphs 32 to 34.
36. A method for treating and/or preventing a disease, which comprises the step of administering a cell composition according to paragraph 35 to a subject.
37. A method according to paragraph 36, wherein the disease is a cancer.
38. A cell composition according to paragraph 35 for use in treating and/or preventing a disease.
39 The use of a cell according to any of paragraphs 32 to 34 in the manufacture of a medicament for treating and/or preventing a disease.
40. A nucleic acid construct which comprises a nucleic acid sequence encoding a dominant negative TGFβ receptor; a nucleic acid sequence encoding a IL7; and a nucleic acid sequence encoding CCL19
41. A nucleic acid construct according to paragraph 40, which has the structure:
dnTGFβR-coexpr1-IL7-coexpr2-CCL19;
dnTGFβR-coexpr1-CCL19-coexpr2-IL7;
IL7-coexpr1-CCL19-coexpr2-dnTGFβR;
IL7-coexpr1-dnTGFβR-CCL19-coexpr2;
CCL19-coexpr1-IL7-coexpr2-dnTGFβR; or
CCL19-coexpr1-dnTGFβR-coexpr2-IL7
   in which:
“dnTGFβR” is a nucleic acid sequence encoding a dominant negative TGFβ receptor;
“IL7” is a nucleic acid sequence encoding IL7
“CCL19” is a nucleic acid sequence encoding CCL19
“coexpr1” and “coexpr2”, which may be the same or different, are nucleic acid sequences enabling coexpression of the three polypeptides as separate entities.
42. A nucleic acid construct according to paragraph 40 which also comprises a nucleic acid sequence encoding a CAR.
43. A nucleic acid construct according to paragraph 3, which has the structure:
CAR-coexpr1-dnTGFβR-coexpr2-IL7-coexpr3-CCL19;
   in which:
dnTGFβR” is a nucleic acid sequence encoding a dominant negative TGFβ receptor;
“IL7” is a nucleic acid sequence encoding IL7
“CCL19” is a nucleic acid sequence encoding CCL19
“CAR” is a nucleic acid sequence encoding a chimeric antigen receptor
“coexpr1”, “coexpr2” and “coexpr3”, which may be the same or different, are nucleic acid sequences enabling coexpression of the four polypeptides as separate entities.
44. A nucleic acid construct according to any of paragraphs 3 to 5, wherein the CAR binds GD2.
45. A vector comprising a nucleic acid construct according to any of paragraphs 40 to 44.
46. A kit of vectors comprising a first vector which comprises a nucleic acid sequence encoding IL7 and a nucleic acid sequence encoding CCL19; and a second vector which comprises a nucleic acid sequence encoding a dominant negative TGFβ receptor.
47. A kit of vectors according to paragraph 46, in which both vectors also comprise a nucleic acid sequence encoding a chimeric antigen receptor (CAR).
48. A kit of vectors according to paragraph 47, wherein the CAR encoded by the first vector is the same as the CAR encoded by the second vector.
49. A kit of vectors according to paragraph 48, wherein the CAR binds GD2.
50. A method for making a cell composition which comprises step of transducing a population of cells with a vector according to paragraph 45 or a kit of vectors according to any of paragraphs 46 to 49.
51. A cell which co-expresses dominant negative TGFβ receptor, IL7 and CCL19.
52. A cell according to paragraph 51, which also expresses one or more chimeric antigen receptor(s) (CAR(s)).
53. A cell composition made by a method according to paragraph 46 or comprising a plurality of cells according to paragraph 51 or 52.
54. A method for treating and/or preventing a disease, which comprises the step of administering a cell composition according to paragraph 49 to a subject.
55. A method according to paragraph 54, wherein the disease is a cancer.
56. A cell composition according to paragraph 53 for use in treating and/or preventing a disease.
57. The use of a cell according to paragraph 51 or 52 in the manufacture of a medicament for treating and/or preventing a disease.

In the above paragraphs and the below claims, polypeptide-encoding elements of a nucleic acid construct or vector, such as “dnSHP”, “dnTGFβR”, “IL7”, “CCL19” and “CAR” may be in any order in the construct.

The following detailed description, as it relates to nucleic acid and polypeptide sequences, polypeptide components, vectors, cells methods etc applies equally to the aspects laid out in the above paragraphs as to the aspects of the invention in the claims.

### DETAILED DESCRIPTION

The present invention relates to a method for making a cell composition which comprises step of transducing a population of cells with a mixture of at least two viral vectors.

The viral vectors may, for example, be retroviral vectors or lentiviral vectors.

Retroviruses are double stranded RNA enveloped viruses mainly characterized by the ability to “reverse-transcribe” their genome from RNA to DNA. Virions measure 100-120 nm in diameter and contain a dimeric genome of identical positive RNA strands complexed with the nucleocapsid proteins. The genome is enclosed in a proteic capsid that also contains enzymatic proteins, namely the reverse transcriptase, the integrase and proteases, required for viral infection. The matrix proteins form a layer outside the capsid core that interacts with the envelope, a lipid bilayer derived from the host cellular membrane, which surrounds the viral core particle. Anchored on this bilayer, are the viral envelope glycoproteins responsible for recognizing specific receptors on the host cell and initiating the infection process. Envelope proteins are formed by two subunits, the transmembrane (TM) that anchors the protein into the lipid membrane and the surface (SU) which binds to the cellular receptors.

Based on the genome structure, retroviruses are classified into simple retroviruses, such as MLV and murine leukemia virus; or complex retroviruses, such as HIV and EIAV. Retroviruses encode four genes: gag (group specific antigen), pro (protease), pol (polymerase) and env (envelope). The gag sequence encodes the three main structural proteins: the matrix protein, nucleocapsid proteins, and capsid protein. The pro sequence encodes proteases responsible for cleaving Gag and Gag-Pol during particle assembly, budding and maturation. The pol sequence encodes the enzymes reverse transcriptase and integrase, the former catalyzing the reverse transcription of the viral genome from RNA to DNA during the infection process and the latter responsible for integrating the proviral DNA into the host cell genome. The env sequence encodes for both SU and TM subunits of the envelope glycoprotein. Additionally, retroviral genome presents non-coding cis-acting sequences such as: two LTRs (long terminal repeats), which contain elements required to drive gene expression, reverse transcription and integration into the host cell chromosome; a sequence named packaging signal (w) required for specific packaging of the viral RNA into newly forming virions; and a polypurine tract (PPT) that functions as the site for initiating the positive strand DNA synthesis during reverse transcription. In addition to gag, pro, pol and env, complex retroviruses, such as lentiviruses, have accessory genes including vif, vpr, vpu, nef, tat and rev that regulate viral gene expression, assembly of infectious particles and modulate viral replication in infected cells.

During the process of infection, a retrovirus initially attaches to a specific cell surface receptor. On entry into the susceptible host cell, the retroviral RNA genome is then copied to DNA by the virally encoded reverse transcriptase which is carried inside the parent virus. This DNA is transported to the host cell nucleus where it subsequently integrates into the host genome. At this stage, it is typically referred to as the provirus. The provirus is stable in the host chromosome during cell division and is transcribed like other cellular proteins. The provirus encodes the proteins and packaging machinery required to make more virus, which can leave the cell by a process known as “budding”.

When enveloped viruses, such as retrovirus and lentivirus, bud out of the host cells, they take part of the host cell lipidic membrane. In this way, host-cell derived membrane proteins become part of the retroviral particle. The present invention utilises this process in order to introduce proteins of interest into the envelope of the viral particle.

### Viral Vectors

Retroviruses and lentiviruses may be used as a vector or delivery system for the transfer of a nucleic acid sequence, or a plurality of nucleic acid sequences, to a target cell. The transfer can occur in vitro, ex vivo or in vivo. When used in this fashion, the viruses are typically called viral vectors.

Gamma-retroviral vectors, commonly designated retroviral vectors, were the first viral vector employed in gene therapy clinical trials in 1990 and are still one of the most used. More recently, the interest in lentiviral vectors, derived from complex retroviruses such as the human immunodeficiency virus (HIV), has grown due to their ability to transduce non-dividing cells. The most attractive features of retroviral and lentiviral vectors as gene transfer tools include the capacity for large genetic payload (up to 9 kb), minimal patient immune response, high transducing efficiency in vivo and in vitro, and the ability to permanently modify the genetic content of the target cell, sustaining a long-term expression of the delivered gene.

The retroviral vector can be based on any suitable retrovirus which is able to deliver genetic information to eukaryotic cells. For example, the retroviral vector may be an alpharetroviral vector, a gammaretroviral vector, a lentiviral vector or a spumaretroviral vector. Such vectors have been used extensively in gene therapy treatments and other gene delivery applications.

The viral vector of the present invention may be a retroviral vector, such as a gamma-retroviral vector. The viral vector may be based on human immunodeficiency virus.

The viral vector of the present invention may be a lentiviral vector. The vector may be based on a non-primate lentivirus such as equine infectious anemia virus (EIAV).

### Nucleic Acid Sequences and Constructs

In the mixture of viral vectors used in the method of the present invention, each vector may comprise one or more nucleic acid sequences. For example, one or more of the vectors in the mixture may comprise a nucleic acid construct comprising a plurality of nucleic acid sequences which are co-expressed. The nucleic acid construct may, for example, be bicistronic or tri-cistronic. The nucleic acid construct may comprise 2, 3, 4 or 5 transgenes.

The nucleic acid sequences in the nucleic acid construct may be separated by a “coexpression” sequence which enables the two or more polypeptides, once translated, to be expressed separately in or on the cell.

The coexpression sequence may encode a cleavage site, such that the nucleic acid construct produces comprises two or more polypeptides joined by a cleavage site(s). The cleavage site may be self-cleaving, such that when the polypeptide is produced, it is immediately cleaved into individual polypeptides without the need for any external cleavage activity.

The cleavage site may be any sequence which enables the two or more polypeptides to become separated.

The term “cleavage” is used herein for convenience, but the cleavage site may cause the polypeptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may be a furin cleavage site.

Furin is an enzyme which belongs to the subtilisin-like proprotein convertase family. The members of this family are proprotein convertases that process latent precursor proteins into their biologically active products. Furin is a calcium-dependent serine endoprotease that can efficiently cleave precursor proteins at their paired basic amino acid processing sites. Examples of furin substrates include proparathyroid hormone, transforming growth factor beta 1 precursor, proalbumin, pro-beta-secretase, membrane type-1 matrix metalloproteinase, beta subunit of pro-nerve growth factor and von Willebrand factor. Furin cleaves proteins just downstream of a basic amino acid target sequence (canonically, Arg-X-(Arg/Lys)-Arg′ (SEQ ID No. 58)) and is enriched in the Golgi apparatus.

The cleavage site may be a Tobacco Etch Virus (TEV) cleavage site.

TEV protease is a highly sequence-specific cysteine protease which is chymotrypsin-like proteases. It is very specific for its target cleavage site and is therefore frequently used for the controlled cleavage of fusion proteins both in vitro and in vivo. The consensus TEV cleavage site is ENLYFQ\S (where ‘\’ denotes the cleaved peptide bond) (SEQ ID No. 59). Mammalian cells, such as human cells, do not express TEV protease. Thus in embodiments in which the present nucleic acid construct comprises a TEV cleavage site and is expressed in a mammalian cell-exogenous TEV protease must also expressed in the mammalian cell.

The cleavage site may encode a self-cleaving peptide.

A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al (2001) as above).

The cleavage site may comprise the 2A-like sequence shown as SEQ ID No. 9.

SEQ ID No. 9
RAEGRGSLLTCGDVEENPGP

The present invention provides a nucleic acid construct which comprises a nucleic acid sequence encoding a dominant negative SHP-1 or SHP-2 and a nucleic acid sequence encoding a dominant negative TGFβ receptor.

Dominant negative SHP-1 or SHP-2 and TGFβ receptors are described in more detail below.

The nucleic acid construct may have the structure:
dnSHP-coexpr-dnTGFβR, or
dnTGFβR-coexpr-dnSHP
   in which:
dnSHP is a nucleic acid sequence encoding dominant negative SHP-1 or SHP-2
“coexpr” is a nucleic acid sequences enabling coexpression of the two polypeptides as separate entities
dnTGFβR is a dominant negative TGFβ receptor.

The nucleic acid construct may also comprise a nucleic acid sequence encoding a CAR. In which case the nucleic acid construct may have the structure:
CAR-coexpr1-dnSHP-coexpr2-dnTGFβR
CAR-coexpr1-dnTGFβR-coexpr2-dnSHP
dnTGFβR-coexpr1-CAR-coexpr2-dnSHP
dnTGFβR-coexpr1-dnSHP-coexpr2-CAR
dnSHP-coexpr1-dnTGFβR-coexpr2-CAR or
dnSHP-coexpr1-CAR-coexpr2-dnTGFβR
   in which:
dnSHP is a nucleic acid sequence encoding dominant negative SHP-2
“coexpr1” and “coexpr2” which may be the same or different, are nucleic acid sequences enabling coexpression of the three polypeptides as separate entities dnTGFβR is a dominant negative TGFβ receptor; and
CAR is a nucleic acid sequence encoding a chimeric antigen receptor.

### Suicide Gene

A nucleic acid construct may also comprise a nucleic acid encoding a suicide gene.

Since T-cells engraft and are autonomous, a means of selectively deleting CAR T-cells in patients is desirable. Suicide genes are genetically encodable mechanisms which result in selective destruction of infused T-cells in the face of unacceptable toxicity. The earliest clinical experience with suicide genes is with the Herpes Virus Thymidine Kinase (HSV-TK) which renders T-cells susceptible to Ganciclovir. HSV-TK is a highly effective suicide gene. However, pre-formed immune responses may restrict its use to clinical settings of considerable immunosuppression such as haploidentical stem cell transplantation. Inducible Caspase 9 (iCasp9) is a suicide gene constructed by replacing the activating domain of Caspase 9 with a modified FKBP12. iCasp9 is activated by an otherwise inert small molecular chemical inducer of dimerization (CID). iCasp9 has been recently tested in the setting of haploidentical HSCT and can abort GvHD. Both iCasp9 and HSV-TK are intracellular proteins, so when used as the sole transgene, they have been co-expressed with a marker gene to allow selection of transduced cells.

WO2016/135470 describes a suicide gene which also comprises Caspase 9 but can be induced to dimerise using rapamycin or a rapamycin analog.

This suicide gene, sometimes termed Rapcasp9 or Rapacasp9, has the amino acid sequence shown as SEQ ID No. 80.

### SEQ ID No. 80 (Rapcasp9)

WO2013/153391 describes a marker/suicide gene known as RQR8 which can be detected with the antibody QBEnd10 and expressing cells lysed with the therapeutic antibody Rituximab.

The sort/suicide gene RQR8 has the amino acid sequence shown as SEQ ID No. 79.

(RQR8)
SEQ ID No. 79
CPYSNPSLCSGGGGSELPTQGTFSNVSTNVSPAKPTTTACPYSNPSLCS
GGGGSPAPRPPTPAPTIASQPLSLRPEACRPAAGGAVHTRGLDFACDIY
IWAPLAGTCGVLLLSLVITLYCNHRNRRRVCKCPRPVV

Including a suicide gene in one or more of the vectors in the viral vector composition of the invention enables the selective ablation of a proportion of transduced cells within the subject.

For example, for two vectors A and B, transduced cells will be a mixture of cells transduced with vector A alone, cells transduced with vector B alone, and cells transduced with both vectors A and B. If vector A expresses or co-expresses a suicide gene, activating the suicide gene will cause the deletion of cells transduced with vector A alone, or with vectors A and B, but cells transduced with vector B alone will be spared.

This is particularly useful where one vector in the mixture encodes a potentially dangerous or toxic gene. If a suicide gene is included on the cassette for that vector, then in the event of an unacceptable immunological or toxic event in the patient, cells expressing the gene in question can be selectively deleted by triggering the suicide gene. Cells expressing other vector combinations which do not include the potentially dangerous gene/suicide gene combination are spared and can continue their therapeutic effect.

For example, a suicide gee may be included in a vector which expresses an immunomodulatory cytokine such as IL-12 or a constitutively active cytokine receptor (see below).

### Viral Vector Composition

The present invention provides a viral vector composition which comprises a mixture viral vectors. The composition may be made by simply mixing two of more viral vectors. The composition may comprise between 2 and 10 viral vectors, for example, 2, 3, 4, 5 or 6 viral vectors.

The viral vectors in the mixture may each comprise one or more transgenes. Two or more viral vectors in the composition may overlap in one or more transgenes. For example, two viral vectors in the composition may comprise a nucleic acid sequence encoding the same CAR, but may differ in the presence or type of activity modulator(s) encoded by other nucleic acid sequences.

One or more of the viral vector(s) in the composition may comprises a nucleic acid sequence encoding a dominant negative SHP-1 or SHP-2. One or more viral vector(s) in the composition may comprise a nucleic acid sequence encoding a dominant negative TGFβ receptor. One or more viral vectors in the composition may comprise a nucleic acid sequence encoding a chimeric antigen receptor.

The viral vector composition may comprise a vector which comprises a nucleic acid sequence encoding a dominant negative SHP-1 or SHP-2 and a nucleic acid sequence encoding a dominant negative TGFβ receptor.

The viral vector composition may comprise plurality of vectors, each of which encode different activity modulator(s) or activity modulator combinations.

### Chimeric Antigen Receptor

In the method of the present invention at least one vector in the mixture of viral vectors may comprise a nucleic acid sequence which encodes a chimeric antigen receptor (CAR).

### Chimeric Antigen Receptors (Cars)

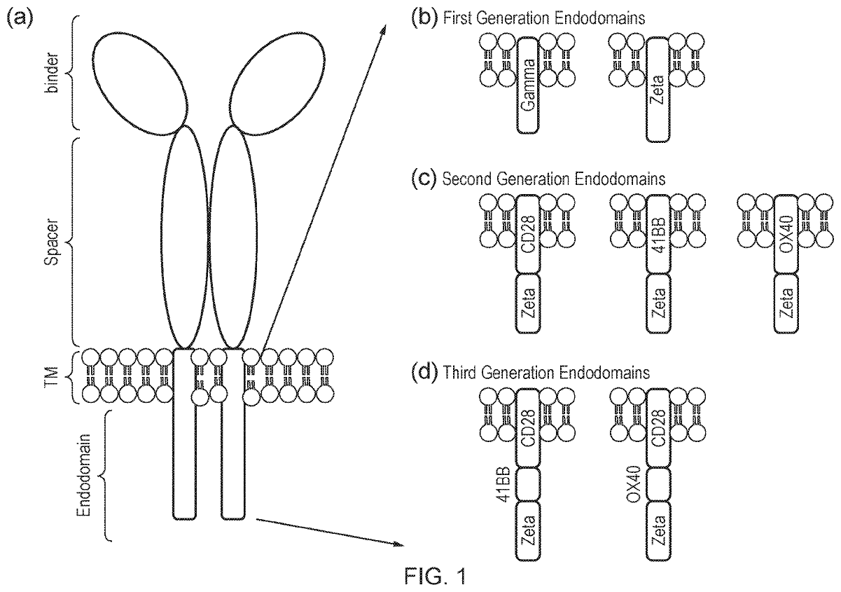

CARs, which are shown schematically in FIG. 1, are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A common spacer domain used is the Fc of IgG1. More compact spacers can suffice e.g. the stalk from CD8a and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal-namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

CAR-encoding nucleic acids may be transferred to T cells using, for example, retroviral or lentiviral vectors to generate cancer-specific T cells for adoptive cell transfer. When the CAR binds the target-antigen, this results in the transmission of an activating signal to the T-cell it is expressed on. Thus, the CAR directs the specificity and cytotoxicity of the T cell towards tumour cells expressing the targeted antigen.

### Tandem Cars (TanCARs)

Bispecific CARs, known as tandem CARs or TanCARs, have been developed to target two or more cancer specific markers simultaneously. In a TanCAR, the extracellular domain comprises two antigen binding specificities in tandem, joined by a linker. The two binding specificities (scFvs) are thus both linked to a single transmembrane portion: one scFv being juxtaposed to the membrane and the other being in a distal position. When a TanCAR binds either or both of the target antigens, this results in the transmission of an activating signal to the cell it is expressed on.

Grada et al (2013, Mol Ther Nucleic Acids 2: e105) describes a TanCAR which includes a CD19-specific scFv, followed by a Gly-Ser linker and then a HER2-specific scFv. The HER2-scFv was in the juxta-membrane position, and the CD19-scFv in the distal position. The TanCAR was shown to induce distinct T cell reactivity against each of the two tumour restricted antigens. This arrangement was chosen because the respective lengths of HER2 (632 aa/125 Å) and CD19 (280 aa, 65 Å) lends itself to that spatial arrangement. It was also known that the HER2 scFv bound the distal-most 4 loops of HER2.

### Antigen Binding Domain

The antigen binding domain is the portion of CAR which recognizes antigen. Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain antibody; an artificial single binder such as a Darpin (designed ankyrin repeat protein); or a single-chain derived from a T-cell receptor.

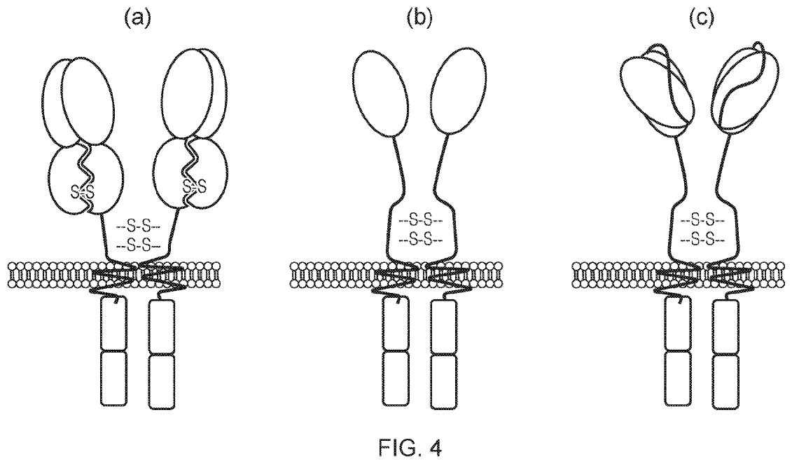

In a classical CAR, the antigen-binding domain comprises: a single-chain variable fragment (scFv) derived from a monoclonal antibody (see FIG. 4c). CARs have also been produced with domain antibody (dAb) or VHH antigen binding domains (see FIG. 4b) or which comprise a Fab fragment of, for example, a monoclonal antibody (see FIG. 4a). A FabCAR comprises two chains: one having an antibody-like light chain variable region (VL) and constant region (CL); and one having a heavy chain variable region (VH) and constant region (CH). One chain also comprises a transmembrane domain and an intracellular signalling domain. Association between the CL and CH causes assembly of the receptor.

The two chains of a Fab CAR may have the general structure:
VH-CH—spacer—transmembrane domain—intracellular signalling domain; and
VL-CL or
VL-CL—spacer—transmembrane domain—intracellular signalling domain; and
VH-CH

For Fab-type chimeric receptors, the antigen binding domain is made up of a VH from one polypeptide chain and a VL from another polypeptide chain.

The polypeptide chains may comprise a linker between the VH/VL domain and the CH/CL domains. The linker may be flexible and serve to spatially separate the VH/VL domain from the CH/CL domain.

The antigen-binding domain of the CAR may bind a tumour associated antigen. Various tumour associated antigens (TAA) are known, for example as shown in the following Table 1.

TABLE 1

| Cancer type | TAA |
|---|---|
| Diffuse Large B-cell Lymphoma | CD19, CD20, CD22 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

The or each CAR may bind one of the following target antigens: CD19, CD22, BCMA, PSMA, GD2, CD79 or FCRL5.

### CD19

An antigen binding domain of a CAR which binds to CD19 may comprise a sequence derived from one of the CD19 binders shown in Table 2.

TABLE 2

| Binder | References |
|---|---|
| HD63 | Pezzutto (Pezzutto, A. et al. J. Immunol. |
| | Baltim. Md 1950 138, 2793-2799 (1987) |
| 4g7 | Meeker et al (Meeker, T. C. et al. Hybridoma 3, |
| | 305-320 (1984) |
| Fmc63 | Nicholson et al (Nicholson, I. C. et al. Mol. |
| | Immunol. 34, 1157-1165 (1997) |
| B43 | Bejcek et al (Bejcek, B. E. et al. Cancer Res. 55, |
| | 2346-2351 (1995) |
| SJ25C1 | Bejcek et al (1995, as above) |
| BLY3 | Bejcek et al (1995, as above) |
| B4, or re-surfaced, | Roguska et al (Roguska, M. A. et al. Protein |
| or humanized B4 | Eng. 9, 895-904 (1996) |
| HB12b, optimized | Kansas et al (Kansas, G. S. & Tedder, T. F. J. |
| and humanized | Immunol. Baltim. Md 1950 147, 4094-4102 (1991); |
| | Yazawa et al (Yazawa et al Proc. Natl. Acad. Sci. |
| | U.S.A. 102, 15178-15183 (2005); Herbst et al |
| | (Herbst, R. et al. J. Pharmacol. Exp. Ther. 335, |
| | 213-222 (2010) |

Alternatively a CAR which binds CD19 may have an antigen-binding domain which comprises:

a) a heavy chain variable region (VH) having
complementarity determining regions (CDRs) with
the following sequences:
CDR1
(SEQ ID No. 1)
GYAFSSS;
CDR2
(SEQ ID No. 2)
YPGDED;
CDR3
(SEQ ID No. 3)
SLLYGDYLDY;
and
b) a light chain variable region (VL) having
CDRs with the following sequences:
CDR1
(SEQ ID No. 4)
SASSSVSYMH;
CDR2
(SEQ ID No. 5)
DTSKLAS
CDR3
(SEQ ID No. 6)
QQWNINPLT.

The antigen binding domain may comprise a VH domain having the sequence shown as SEQ ID No. 7 and a VL domain having the sequence shown as SEQ ID No 8.

VH sequence
SEQ ID No. 7
QVQLQQSGPELVKPGASVKISCKASGYAFSSSWMNWVKQRPGKGLEWIG
RIYPGDEDTNYSGKFKDKATLTADKSSTTAYMQLSSLTSEDSAVYFCAR
SLLYGDYLDYWGQGTTLTVSS
VL sequence
SEQ ID No 8
QIVLTQSPAIMSASPGEKVTMTCSASSSVSYMHWYQQKSGTSPKRWIYD
TSKLASGVPDRFSGSGSGTSYFLTINNMEAEDAATYYCQQWNINPLTFG
AGTKLELKR

### CD22

A CAR which binds to CD22 may have an antigen domain derived from m971, HA22 or BL22 as described by Haso et al. (Blood; 2013; 121 (7)).

Alternatively, a CAR which binds CD22 may have an antigen binding domain as described in United Kingdom application No. 1809773.3, such as one which comprises:

a) a heavy chain variable region (VH) having
complementarity determining regions (CDRs) with
the following sequences:
CDR1
(SEQ ID No. 10)
NFAMA
CDR2
(SEQ ID No. 11)
SISTGGGNTYYRDSVKG
CDR3Q
(SEQ ID No. 12)
RNYYDGSYDYEGYTMDA;
and
b) a light chain variable region (VL) having
complementarity determining regions (CDRs) with
the following sequences:
CDR1
(SEQ ID No. 13)
RSSQDIGNYLT
CDR2
(SEQ ID No. 14)
GAIKLED
CDR3
(SEQ ID No. 15)
LQSIQYP

The antigen binding domain of a CD22 CAR may comprise a VH domain having the sequence shown as SEQ ID No. 16; and a VL domain having the sequence shown as SEQ ID No. 17.

SEQ ID No. 16
EVQLVESGGGLVQPGRSLKLSCAASGFTFSNFAMAWVRQPPTKGLEWVA
SISTGGGNTYYRDSVKGRFTISRDDAKNTQYLQMDSLRSEDTATYYCAR
QRNYYDGSYDYEGYTMDAWGQGTSVTVSS
SEQ ID No. 17
DIQMTQSPSSLSASLGDRVTITCRSSQDIGNYLTWFQQKVGRSPRRMIY
GAIKLEDGVPSRFSGSRSGSDYSLTISSLESEDVADYQCLQSIQYPFTF
GSGTKLEIK

### BCMA

A number of BCMA-targeted CARs are in clinical development, including bb2121, LCAR-B38M, MCARH171, JCARH125, P-BCMA-101, FCARH143, bb21217 and CT053.

WO2015/052538 describes a BCMA targeted CAR in which the antigen-binding domain is derived from a proliferation inducing ligand (APRIL) which is a natural ligand for BCMA.

UK Patent application No. 1815775.0 describes the VH and VL domains for 14 BCMA binding domains and their use in CARs.

### PSMA

T-cells expressing CARs specific for prostate-specific membrane antigen (PSMA) are currently in clinical trial for the treatment of prostate cancer (Junhans et al (2016) Prostate 76:1257-1270).

### GD2

CARs have been developed which bind disialoganglioside (GD2) a sialic acid-containing glycosphingolipid. Such CARs may, for example, be based on the GD2 binder 14g2a, or huK666 as described in WO2015/132604.

A CAR which binds GD2 may have an antigen-binding domain which comprises:

a) a heavy chain variable region (VH) having
complementarity determining regions (CDRs) with
the following sequences:
CDR1
(SEQ ID No. 71)
SYNIH;
CDR2
(SEQ ID No. 72)
VIWAGGSTNYNSALMS
CDR3
(SEQ ID No. 73)
RSDDYSWFAY;
and
b) a light chain variable region (VL) having
CDRs with the following sequences:
CDR1
(SEQ ID No. 74)
RASSSVSSSYLH;
CDR2
(SEQ ID No. 75)
STSNLAS
CDR3
(SEQ ID No. 76)
QQYSGYPIT.

The GD2 binding domain may comprise a VH domain having the sequence shown as SEQ ID No. 77; and/or a VL domain having the sequence shown as SEQ ID No 78.

(Humanised KM666 VH sequence)
SEQ ID No. 77
QVQLQESGPGLVKPSQTLSITCTVSGFSLASYNIHWVRQPPGKGLEWLG
VIWAGGSTNYNSALMSRLTISKDNSKNQVFLKMSSLTAADTAVYYCAKR
SDDYSWFAYWGQGTLVTVSS
(Humanised KM666 VH sequence)
SEQ ID No. 78
ENQMTQSPSSLSASVGDRVTMTCRASSSVSSSYLHWYQQKSGKAPKVWI
YSTSNLASGVPSRFSGSGSGTDYTLTISSLQPEDFATYYCQQYSGYPIT
FGQGTKVEIK

### FCRL5

Commercially available monoclonal antibodies against FcRL5 are known, such as CD307e (ThermoFisher) and REA391 (Miltenyi Biotec).

WO2016090337 describes several scFv-type antigen-binding domains which bind FcRL5.

UK Patent application No. 1815775.0 describes anti-FCRL5 CARs.

### CD79

A number of anti-CD79 antibodies have been previously described, such as JCB117, SN8, CB3.1, 2F2 (Polatuzumab).

United Kingdom application No. 1807870.9 describes various CD79 CARs.

Where the composition of viral vectors includes more than one vector comprising a nucleic acid sequence encoding a CAR, the CARs may have different antigen binding domains. The CARs may recognise different antigens, or the CARs may bind the same antigen but have different antigen-binding domains. CARs which bind the same antigen but have different antigen-binding domains may bind to different epitopes of the antigen and/or may have different affinities and/or on or off rates.

The affinity of a CAR for the target antigen and/or its on and off rate, can affect the capacity of a CAR to kill target cells. For example, it is reported in US 2018/0064785 that a CAR derived from an antibody with a fast on-rate and a fast off-rate allows a CAR T-cell to better serially kill target cells. By giving a patient a CAR-T cell composition which comprises a plurality of CARs against the target antigen, the CAR with the antigen-binding domain best suited to kill target cells in the patient or at a particular site in the patient will receive activation/survival/proliferation signals and will prevail. The composition of the invention gives flexibility in this regard and even allows CAR-T cell subpopulations having different CARs to “win out” at different sites within the same patient.

### Intracellular T Cell Signaling Domain (Endodomain)

The CAR may comprise or associate with an activating endodomain: the signal-transmission portion of the CAR. After antigen recognition, receptors cluster and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signaling may be needed. For example, chimeric CD28 and OX40 can be used with CD3-Zeta to transmit a proliferative/survival signal, or all three can be used together.

The endodomain of the CAR may comprise the CD28 endodomain and OX40 and CD3-Zeta endodomain.

The endodomain may comprise:
(i) an ITAM-containing endodomain, such as the endodomain from CD3 zeta; and/or
(ii) a co-stimulatory domain, such as the endodomain from CD28; and/or
(iii) a domain which transmits a survival signal, for example a TNF receptor family endodomain such as OX-40 or 4-1BB.

An endodomain which contains an ITAM motif can act as an activation endodomain in this invention. Several proteins are known to contain endodomains with one or more ITAM motifs. Examples of such proteins include the CD3 epsilon chain, the CD3 gamma chain and the CD3 delta chain to name a few. The ITAM motif can be easily recognized as a tyrosine separated from a leucine or isoleucine by any two other amino acids, giving the signature YxxL/I (SeQ ID NO. 60). Typically, but not always, two of these motifs are separated by between 6 and 8 amino acids in the tail of the molecule (YxxL/Ix (6-8) YxxL/I). Hence, one skilled in the art can readily find existing proteins which contain one or more ITAM to transmit an activation signal. Further, given the motif is simple and a complex secondary structure is not required, one skilled in the art can design polypeptides containing artificial ITAMs to transmit an activation signal (see WO 2000/063372, which relates to synthetic signalling molecules).

A number of systems have been described in which the antigen recognition portion of the CAR is on a separate molecule from the signal transmission portion, such as those described in WO015/150771; WO2016/124930 and WO2016/030691. One or more of the viral vectors used in the method of the invention may encode such a “split CAR”. Alternatively one vector may comprise a nucleic acid sequence encoding the antigen recognition portion and one vector may comprise a nucleic acid sequence encoding the intracellular signalling domain.

Where the composition of viral vectors includes more than one vector comprising a nucleic acid sequence encoding a CAR, the CARs may have different endodomains or different endodomain combinations. For example, one CAR may be a second generation CAR and one CAR may be a third generation CAR. Alternatively, both CARs may be a second generation CAR but may have different co-stimulatory domains. For example, different second generation CAR signalling domains include: 41BB-CD3ζ; OX40-CD3ζ and CD28-CD3ζ.

### Signal Peptide

One or more nucleic acid sequences in the vector composition may encode a signal peptide so that when the CAR or activity modulator is expressed inside a cell, the nascent protein is directed to the endoplasmic reticulum and subsequently to the cell surface, where it is expressed (or secreted).

The core of the signal peptide may contain a long stretch of hydrophobic amino acids that tends to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

A CAR may have the general formula:

Signal peptide−antigen binding domain−spacer domain−transmembrane domain−intracellular T cell signaling domain (endodomain).

### Spacer

The CAR may comprise a spacer sequence to connect the antigen binding domain with the transmembrane domain and spatially separate the antigen binding domain from the endodomain. A flexible spacer allows to the antigen binding domain to orient in different directions to enable antigen binding.

The spacer sequence may, for example, comprise an IgG1 Fc region, an IgG1 hinge or a CD8 stalk, or a combination thereof. The spacer may alternatively comprise an alternative sequence which has similar length and/or domain spacing properties as an IgG1 Fc region, an IgG1 hinge or a CD8 stalk.

Where the composition of viral vectors includes more than one vector comprising a nucleic acid sequence encoding a CAR, the CARs may have different spacers.

### Or Gates

A cell composition of the present invention may comprise two or more CARs. This may be as a result of transduction with two or more vectors, each comprising a nucleic acid sequence encoding a CAR; or it may be as a result of transduction with a single vector which comprises a nucleic acid construct encoding two or more CARs.

A CAR may be used in a combination with one or more other activatory or inhibitory chimeric antigen receptors. For example, they may be used in combination with one or more other CARs in a “logic-gate”, a CAR combination which, when expressed by a cell, such as a T cell, are capable of detecting a particular pattern of expression of at least two target antigens. If the at least two target antigens are arbitrarily denoted as antigen A and antigen B, the three possible options are as follows:
“OR GATE”—T cell triggers when either antigen A or antigen B is present on the target cell
“AND GATE”—T cell triggers only when both antigens A and B are present on the target cell
“AND NOT GATE”—T cell triggers if antigen A is present alone on the target cell, but not if both antigens A and B are present on the target cell

Engineered T cells expressing these CAR combinations can be tailored to be exquisitely specific for cancer cells, based on their particular expression (or lack of expression) of two or more markers.

Such “Logic Gates” are described, for example, in WO2015/075469, WO2015/075470 and WO2015/075470.

An “OR Gate” comprises two or more activatory CARs each directed to a distinct target antigen expressed by a target cell. The advantage of an OR gate is that the effective targetable antigen is increased on the target cell, as it is effectively antigen A+antigen B. This is especially important for antigens expressed at variable or low density on the target cell, as the level of a single antigen may be below the threshold needed for effective targeting by a CAR-T cell. Also, it avoids the phenomenon of antigen escape. For example, some lymphomas and leukemias become CD19 negative after CD19 targeting: using an OR gate which targets CD19 in combination with another antigen provides a “back-up” antigen, should this occur. The “back up” antigen may be CD22, as described in WO2016/102965.

### Activity Modulator

In the method of the present invention at least one vector in the mixture of viral vectors may comprise a nucleic acid sequence which encodes an activity modulator. When this is the case, at least a proportion of the transduced cells in the CAR-expressing cell composition of the invention will express one or more activity modulator(s). An activity modulator is a molecule made by the CAR-expressing cell which modulates the activity of the CAR, of a cell expressing the CAR, or of a target cell.

An activity modulator may be an intracellular molecule, expressed at the cell surface, or secreted by the CAR-expressing cell.

### Modulating the Activity of the Car

### 1. Enhancing ITAM Phosphorylation

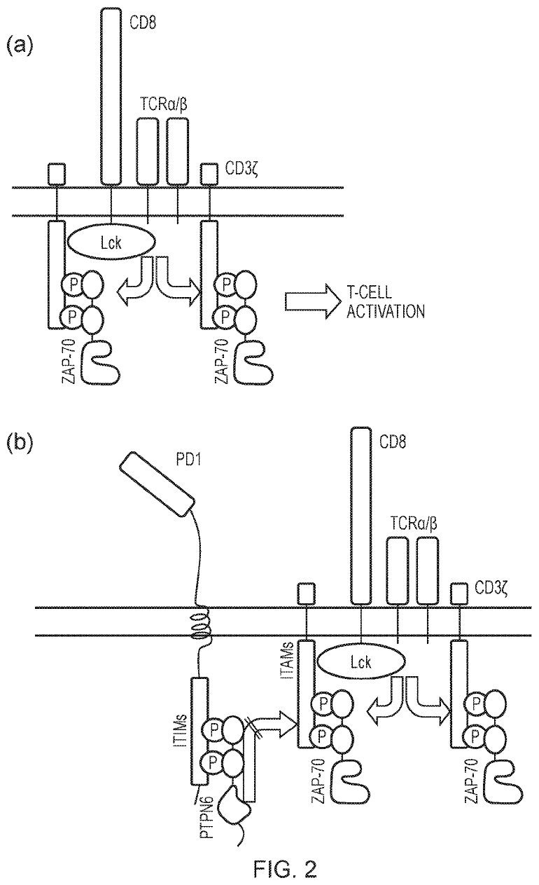

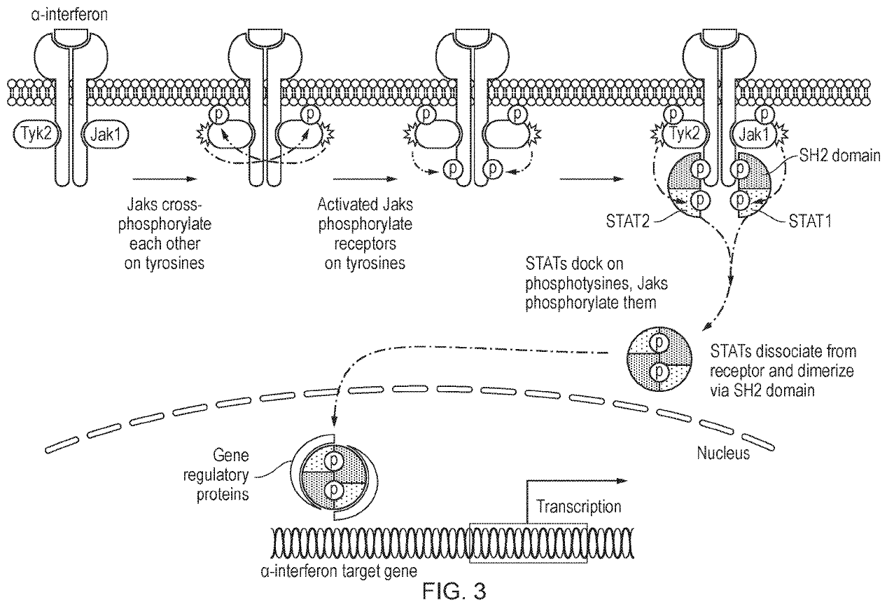

During T cell activation in vivo (illustrated schematically in FIG. 2a), antigen recognition by the T-cell receptor (TCR) results in phosphorylation of Immunoreceptor tyrosine-based activation motifs (ITAMs) on CD3ζ. Phosphorylated ITAMs are recognized by the ZAP70 SH2 domains, leading to T cell activation.

T-cell activation uses kinetic segregation to convert antigen recognition by a TCR into downstream activation signals. Briefly: at the ground state, the signalling components on the T-cell membrane are in dynamic homeostasis whereby dephosphorylated ITAMs are favoured over phosphorylated ITAMs. This is due to greater activity of the transmembrane CD45/CD148 phosphatases over membrane-tethered kinases such as Ick. When a T-cell engages a target cell through a T-cell receptor (or CAR) recognition of cognate antigen, tight immunological synapses form. This close juxtapositioning of the T-cell and target membranes excludes CD45/CD148 due to their large ectodomains which cannot fit into the synapse. Segregation of a high concentration of T-cell receptor associated ITAMs and kinases in the synapse, in the absence of phosphatases, leads to a state whereby phosphorylated ITAMs are favoured. ZAP70 recognizes a threshold of phosphorylated ITAMs and propagates a T-cell activation signal.

The process is essentially the same during CAR-mediated T-cell activation. An activating CAR comprises one or more ITAM(s) in its intracellular signalling domain, usually because the signalling domain comprises the endodomain of CD3ζ. Antigen recognition by the CAR results in phosphorylation of the ITAM(s) in the CAR signalling domain, causing T-cell activation.

As illustrated schematically in FIG. 2b, inhibitory immune-receptors such as PD1 cause the dephosphorylation of phosphorylated ITAMs. PD1 has ITIMs in its endodomain which are recognized by the SH2 domains of molecules such as PTPN6 (SHP-1) and SHP-2. Upon recognition, PTPN6 is recruited to the juxta-membrane region and its phosphatase domain subsequently de-phosphorylates ITAM domains inhibiting immune activation.

An activity modulator capable of modulating the activity of the CAR may be capable of directly or indirectly phosphorylating the ITAM(s) in the CAR signalling domain.

### 1.1 Providing or Recruiting Kinase

For example, the activity modulator may be a membrane targeted molecule which either comprises a kinase domain or is capable of recruiting a separate molecule comprising a kinase domain to the vicinity of the CAR. WO2018/193231 describes various molecules having such a “phosphorylation amplifying endodomain”.

An activity modulator capable of directly phosphorylating ITAMs may comprise a tyrosine kinase domain, such as a kinase domain of a Src family kinase, examples of which include Fyn, Src, Lck or a derivative thereof such as Lck (Y505F). The tyrosine kinase domains of Fyn, Src, Lck and Lck (Y505) are shown below as SEQ ID Nos. 18-21 respectively.

Tyrosine kinase domain Fyn (SEQ ID NO: 18)
LQLIKRLGNGQFGEVWMGTWNGNTKVAIKTLKPGTMSPESFLEEAQIMK
KLKHDKLVQLYAVVSEEPIYIVTEYMNKGSLLDFLKDGEGRALKLPNLV
DMAAQVAAGMAYIERMNYIHRDLRSANILVGNGLICKIADFGLARLIED
NEYTARQGAKFPIKWTAPERALYGRFTIKSDVWSFGILLTELVTKGRVP
YPGMNNREVLEQVERGYRMPCPQDCPISLHELMIHCWKKDPEERPTFEY
LQSFLEDYF
Tyrosine kinase domain of Src (SEQ ID NO: 19)
LRLEVKLGQGCFGEVWMGTWNGTTRVAIKTLKPGTMSPEAFLQEAQVMK
KLRHEKLVQLYAVVSEEPIYIVTEYMSKGSLLDFLKGETGKYLRLPQLV
DMAAQIASGMAYVERMNYVHRDLRAANILVGENLVCKVADFGLARLIED
NEYTARQGAKFPIKWTAPEAALYGRFTIKSDVWSFGILLTELTTKGRVP
YPGMVNREVLDQVERGYRMPCPPECPESLHDLMCQCWRKEPEERPTFEY
LQAFLEDYF
Tyrosine kinase domain of Lck (SEQ ID NO: 20):
LKLVERLGAGQFGEVWMGYYNGHTKVAVKSLKQGSMSPDAFLAEANLMK
QLQHQRLVRLYAVVTQEPIYIITEYMENGSLVDFLKTPSGIKLTINKLL
DMAAQIAEGMAFIEERNYIHRDLRAANILVSDTLSCKIADFGLARLIED
NEYTAREGAKFPIKWTAPEAINYGTFTIKSDVWSFGILLTEIVTHGRIP
YPGMTNPEVIQNLERGYRMVRPDNCPEELYQLMRLCWKERPEDRPTFDY
LRSVLEDFF
Tyrosine kinase domain of Lck_Y505F
(SEQ ID NO: 21)
LKLVERLGAGQFGEVWMGYYNGHTKVAVRSLKQGSMSPDAFLAEANLMK
QLQHQRLVRLYAVVTQEPIYIITEYMENGSLVDFLKTPSGIKLTINKLL
DMAAQIAEGMAFIEERNYIHRDLRAANILVSDTLSCKIADFGLARLIED
NEYTAREGAKFPIKWTAPEAINYGTFTIKSDVWSFGILLTEIVTHGRIP
YPGMTNPEVIQNLERGYRMVRPDNCPEELYQLMRLCWKERPEDRPTFDY
LRSVLEDFF

An activity modulator capable of indirectly phosphorylating ITAMs may comprise the intracellular domain of CD4 or CD8 coreceptor.

As mentioned above, during T-cell activation, the ITAMs of CD3 (or the CAR) are phosphorylated by a Lck and then bound by ZAP70. After ZAP70 binds to CD3, co-receptors CD4 or CD8 become associated with the TCR/CD3 complex and bind to the major compatibility complex (MHC). CD4/CD8 co-receptor association with the complex stabilises the TCR-MHC peptide (MHCp) interaction and the recruited/free Lck continues the phosphorylation of CD3 elements, ZAP70, as well as many other downstream targets.

An activity modulator comprising the cytoplasmic tail of CD4 and CD8 will amplify the signal generated by the CAR by recruiting Lck, which is essential for activating many molecular components of the signaling cascade of an activated T cell. The sequences of the intracellular domain of human CD4 and CD8 are shown below as SEQ ID No. 22 and 23

Cytoplasmic tail of CD4
(SEQ ID NO: 22)
CVRCRHRRRQAERMSQIKRLLSEKKTCQCPHRFQKTCSPI
Cytoplasmic tail of CD8
(SEQ ID NO: 23)
LYCNHRNRRRVCKCPRPVVKSGDKPSLSARYV

An activity modulator which modulated the activity of the CAR may be membrane-tethered. In this respect, such an activity modulator may comprise a transmembrane domain or a myristoylation sequence.

### Modulating the Activity of the Car-T Cell

### 1. Checkpoint Inhibition

An activity modulator capable of modulating the activity of the CAR-expressing cell may block or reduce the inhibition of CAR-mediated T-cell activation mediated by inhibitory immunoreceptors such as CTLA4, PD-1, LAG-3, 2B4 or BTLA 1 (as mentioned above and illustrated schematically in FIG. 2b).

The activity modulator may be an agent, such as an antibody, which binds to an inhibitory immunoreceptor or binds to a ligand for an inhibitory immunoreceptor. The activity modulator may bind to CTLA4, PD-1, LAG-3, 2B4 or BTLA 1, or bind to a ligand for CTLA4, PD-1, LAG-3, 2B4 or BTLA 1.

### PD-1/PD-L1

In the cancer disease state, the interaction of PD-L1 on the tumour cells with PD-1 on a T-cell reduces T-cell activation, as described above, thus hampering the immune system in its efforts to attack the tumour cells. Use of an inhibitor that blocks the interaction of PD-L1 with the PD-1 receptor can prevent the cancer from evading the immune system in this way. Several PD-1 and PD-L1 inhibitors are being trialed within the clinic for use in advanced melanoma, non-small cell lung cancer, renal cell carcinoma, bladder cancer and Hodgkin lymphoma, amongst other cancer types. Some such inhibitors are now approved, including the PD1 inhibitors Nivolumab and Pembrolizumab and the PD-L1 inhibitors Atezolizumab, Avelumab and Durvalumab.

### CTLA4

CTLA4 is a member of the immunoglobulin superfamily that is expressed by activated T cells and transmits an inhibitory signal to T cells. CTLA4 is homologous to the T-cell co-stimulatory protein, CD28, and both molecules bind to CD80 and CD86, also called B7-1 and B7-2 respectively, on antigen-presenting cells. CTLA-4 binds CD80 and CD86 with greater affinity and avidity than CD28 thus enabling it to outcompete CD28 for its ligands. CTLA4 transmits an inhibitory signal to T cells, whereas CD28 transmits a stimulatory signal.

Antagonistic antibodies against CTLA4 include ipilimumab and tremelimumab.

### LAG-3

Lymphocyte-activation gene 3, also known as LAG-3 and CD223, is an immune checkpoint receptor with diverse biologic effects on T-cell function.

Antibodies to LAG3 include relatlimab, which currently in phase 1 clinical testing and a number of others in preclinical development. LAG-3 may be a better checkpoint inhibitor target than CTLA-4 or PD-1 since antibodies to these two checkpoints only activate effector T cells, and do not inhibit Treg activity, whereas an antagonist LAG-3 antibody can both activate T effector cells (by downregulating the LAG-3 inhibiting signal into pre-activated LAG-3+ cells) and inhibit induced (i.e. antigen-specific) Treg suppressive activity. Combination therapies are also ongoing involving LAG-3 antibodies and CTLA-4 or PD-1 antibodies.

### 1.2 Dominant Negative SHP

An activity modulator which blocks or reduces the inhibition mediated by inhibitory immunoreceptors such as CTLA4, PD-1, LAG-3, 2B4 or BTLA 1 may tip the balance of phosphorylation:dephosphorylation at the T-cell:target cell synapse in favour of phosphorylation of ITAMs, leading to T-cell activation. For example, the activity modulator may block or reduce the phosphorylation of ITIMs in the endodomain of inhibitory receptor(s) or may block or reduce the dephosphorylation of ITAMs in the CAR signalling domain by proteins such as SHP-1 and SHP-2.

WO2016/193696 describes various different types of protein capable of modulating the balance of phosphorylation:dephosphorylation at the T-cell:target cell synapse. For example, the activity modulator may comprise a truncated form of SHP-1 or SHP-2 which comprises one or both SH2 domains, but lacks the phosphatase domain. When expressed in a CAR-T cell, these molecules act as dominant negative versions of wild-type SHP-1 and SHP-2 and compete with the endogenous molecule for binding to phosphorylated ITIMs.

The activity modulator may be a truncated protein which comprises an SH2 domain from a protein which binds a phosphorylated immunoreceptor tyrosine-based inhibition motif (ITIM) but lacks a phosphatase domain. The truncated protein may comprise one or both SHP-1 SH2 domain(s) but lack the SHP-1 phosphatase domain. Alternatively the truncated protein may comprise one or both SHP-2 SH2 domain(s) but lack the SHP-2 phosphatase domain.

### SHP-1

Src homology region 2 domain-containing phosphatase-1 (SHP-1) is a member of the protein tyrosine phosphatase family. It is also known as PTPN6.

The N-terminal region of SHP-1 contains two tandem SH2 domains which mediate the interaction of SHP-1 and its substrates. The C-terminal region contains a tyrosine-protein phosphatase domain.

SHP-1 is capable of binding to, and propagating signals from, a number of inhibitory immune receptors or ITIM containing receptors. Examples of such receptors include, but are not limited to, PD1, PDCD1, BTLA4, LILRB1, LAIR1, CTLA4, KIR2DL1, KIR2DL4, KIR2DL5, KIR3DL1 and KIR3DL3.

Human SHP-1 protein has the UniProtKB accession number P29350.

An activity modulator may comprise or consist of the SHP-1 tandem SH2 domain which is shown below as SEQ ID NO: 24.

SHP-1 SH2 complete domain
(SEQ ID NO: 24)
MVRWFHRDLSGLDAETLLKGRGVHGSFLARPSRKNQGDFSLSVRVGDQVT
HIRIQNSGDFYDLYGGEKFATLTELVEYYTQQQGVLQDRDGTIIHLKYPL
NCSDPTSERWYHGHMSGGQAETLLQAKGEPWTFLVRESLSQPGDFVLSVL
SDQPKAGPGSPLRVTHIKVMCEGGRYTVGGLETFDSLTDLVEHFKKTGIE
EASGAFVYLRQPYY

SHP-1 has two SH2 domains at the N-terminal end of the sequence, at residues 4-100 and 110-213. An activity modulator may comprise one or both of the sequences shown as SEQ ID No. 25 and 26.

SHP-1 SH2 1
(SEQ ID NO: 25)
WFHRDLSGLDAETLLKGRGVHGSFLARPSRKNQGDFSLSVRVGDQVTHIR
IQNSGDFYDLYGGEKFATLTELVEYYTQQQGVLQDRDGTIIHLKYPL
SHP-2 SH2 2
(SEQ ID NO. 26)
WYHGHMSGGQAETLLQAKGEPWTFLVRESLSQPGDFVLSVLSDQPKAGPG
SPLRVTHIKVMCEGGRYTVGGLETFDSLTDLVEHFKKTGIEEASGAFVYL
RQPY

The activity modulator may comprise a variant of SEQ ID NO: 24, 25 or 26 having at least 80, 85, 90, 95, 98 or 99% sequence identity, provided that the variant sequence is a SH2 domain sequence has the required properties. In other words, the variant sequence should be capable of binding to the phosphorylated tyrosine residues in the cytoplasmic tail of at least one of PD1, PDCD1, BTLA4, LILRB1, LAIR1, CTLA4, KIR2DL1, KIR2DL4, KIR2DL5, KIR3DL1 or KIR3DL3 which allow the recruitment of SHP-1.

### SHP-2

SHP-2, also known as PTPN11, PTP-1D and PTP-2C is a member of the protein tyrosine phosphatase (PTP) family. Like PTPN6, SHP-2 has a domain structure that consists of two tandem SH2 domains in its N-terminus followed by a protein tyrosine phosphatase (PTP) domain. In the inactive state, the N-terminal SH2 domain binds the PTP domain and blocks access of potential substrates to the active site. Thus, SHP-2 is auto-inhibited. Upon binding to target phospho-tyrosyl residues, the N-terminal SH2 domain is released from the PTP domain, catalytically activating the enzyme by relieving the auto-inhibition.

Human SHP-2 has the UniProtKB accession number P35235-1.

An activity modulator may comprise or consist of the SHP-1 tandem SH2 domain which is shown below as SEQ ID NO: 29. SHP-1 has two SH2 domains at the N-terminal end of the sequence, at residues 6-102 and 112-216. An activity modulator may comprise one or both of the sequences shown as SEQ ID No. 27 and 28.

SHP-2 first SH2 domain
(SEQ ID NO: 27)
WFHPNITGVEAENLLLTRGVDGSFLARPSKSNPGDFTLSVRRNGAVTHIK
IQNTGDYYDLYGGEKFATLAELVQYYMEHHGQLKEKNGDVIELKYPL
SHP-2 second SH2 domain
(SEQ ID No. 28)
WFHGHLSGKEAEKLLTEKGKHGSFLVRESQSHPGDFVLSVRTGDDKGESN
DGKSKVTHVMIRCQELKYDVGGGERFDSLTDLVEHYKKNPMVETLGTVLQ
LKQPL
SHP-2 both SH2 domains
(SEQ ID NO. 29)
WFHPNITGVEAENLLLTRGVDGSFLARPSKSNPGDFTLSVRRNGAVTHIK
IQNTGDYYDLYGGEKFATLAELVQYYMEHHGQLKEKNGDVIELKYPLNCA
DPTSERWFHGHLSGKEAEKLLTEKGKHGSFLVRESQSHPGDFVLSVRTGD
DKGESNDGKSKVTHVMIRCQELKYDVGGGERFDSLTDLVEHYKKNPMVET
LGTVLQLKQPL

The activity modulator may comprise a variant of SEQ ID NO: 27, 28 or 29 having at least 80, 85, 90, 95, 98 or 99% sequence identity, provided that the variant sequence is a SH2 domain sequence has the required properties. In other words, the variant sequence should be capable of binding to the phosphorylated tyrosine residues in the cytoplasmic tail of at least one of PD1, PDCD1, BTLA4, LILRB1, LAIR1, CTLA4, KIR2DL1, KIR2DL4, KIR2DL5, KIR3DL1 or KIR3DL3 which allow the recruitment of SHP-2.

### 3. Cytokines and Cytokine Signalling

An activity modulator may be a cytokine or chemokine. A cytokine may modulate the activity of the CAR-expressing cell and/or modulate the tumour microenvironment.

The activity modulator may be a cytokine or chemokine be selected from: IL12, flexiIL 12, GM-CSF, IL7, IL15, IL21, IL2 and CCL19. In particular, the agent may be IL-7 or IL-12.

IL-7 is a cytokine important for B and T cell development. IL-7 stimulates the differentiation of multipotent (pluripotent) hematopoietic stem cells into lymphoid progenitor cells and stimulates proliferation of all cells in the lymphoid lineage (B cells, T cells and NK cells.

II-7 and the hepatocyte growth factor (HGF) form a heterodimer that functions as a pre-pro-B cell growth-stimulating factor. This cytokine is found to be a cofactor for V(D)J rearrangement of the T cell receptor beta (TCRβ) during early T cell development. The amino acid sequence of human II-7 is available from UniProt (Accession No. P13232)

Interleukin 12 (IL-12) is a potent immunomodulatory cytokine of particular interest for modulating the tumour microenvironment redirecting the immune response against cancer. IL-12 is systemically toxic therefore methods for producing IL-12 locally are of interest. PCT/GB2018/052204 describes a construct where production of an immunomodulatory cytokine such as IL-12 is under the control of a promoter which is activated in the presence of an environmental metabolite, such as kynurenine. Selective production of IL-12 in the presence of an metabolite such as kynurenine enables local production of IL-12 by the CAR- or TCR-expressing cell, only when it is present in the tumour microenvironment.

Alternatively, the immunomodulatory cytokine may be placed downstream of a frame-slip motif or a translational readthrough motif. This provides a means of controlling cytokine expression and reducing the level of expression of cytokine relative to the CAR.

A frame-slip motif (FSM) may comprise a repeat of uracil, thymine or guanine bases, such as the sequence UUUUUUU (SEQ ID No. 61).

A frame-slip motif may also comprise a stop codon. For example, a FSM may comprise one of the following sequences:

(SEQ ID NO. 62)
UUUUUUUGA
(SEQ ID NO. 63)
UUUUUUUAG
(SEQ ID NO. 64)
UUUUUUUAA.

A translational readthrough motif (TRM) may comprise the sequence STOP-CUAG or STOP-CAAUUA, in which “STOP” is a stop codon. For example, a translational readthrough motif may comprise one of the following sequences:

(SEQ ID No. 65)
UGA-CUAG
(SEQ ID No. 66)
UAG-CUAG
(SEQ ID No. 67)
UAA-CUAG
(SEQ ID No. 68)
UGA-CAAUUA
(SEQ ID No. 69)
UAG-CAAUUA
(SEQ ID NO. 70)
UAA-CAAUUA

IL-12 is a heterodimeric cytokine encoded by two separate genes, IL-12A (p35) and IL-12B (p40). The active heterodimer (referred to as ‘p70’), is formed following protein synthesis. The activity modulator may be “flexi-IL12”, which is a fusion between the IL-12a and IL-12ß subunits, joined by a linker. A suitable flexi-IL-12 sequence is shown below as SEQ ID No. 81.

SEQ ID No. 81
(a flexi-IL-12 sequence)
MWIWELKKDVYVVELDWYPDAPGEMVVLTCDTPEEDGITWTLDQSSEVLG
SGKTLTIQVKEFGDAGQYTCHKGGEVLSHSLLLLHKKEDGIWSTDILKDQ
KEPKNKTFLRCEAKNYSGRFTCWWLTTISTDLTFSVKSSRGSSDPQGVTC
GAATLSAERVRGDNKEYEYSVECQEDSACPAAEESLPIEVMVDAVHKLKY
ENYTSSFFIRDIIKPDPPKNLQLKPLKNSRQVEVSWEYPDTWSTPHSYFS
LTFCVQVQGKSKREKKDRVFTDKTSATVICRKNASISVRAQDRYYSSSWS
EWASVPCSGGGGSGGGGSGGGGSRNLPLATPDPGMFPCLHHSQNLLRAVS
NMLQKARQTLEFYPCTSEEIDHEDITKDKTSTVEACLPLELTKNESCLNS
RETSFITNGSCLASRKTSFMMALCLSSIYEDSKMYQVEFKTMNAKLLMDP
KRQIFLDQNMLAVIDELMQALNFNSETVPQKSSLEEPDFYKTKIKLCILL
HAFRIRAVTIDRVMSYLNAS

In SEQ ID No. 81, the serine-glycine linker is in bold and underlined.

The activity modulator is the present invention may be a cytokine which is selectively expressed depending the presence of an environmental metabolite in the microenvironment of the cell. The environmental metabolite may activate the aryl hydrocarbon receptor (AHR). The environmental metabolite may be a tryptophan metabolite such as is kynurenine.

Alternatively, the agent may affect the expression or activity of a cytokine or chemokine. For example, the agent may be a dominant negative version of a cytokine or chemokine. A dominant negative version may, for example, be a mutated or truncated version of the cytokine/chemokine which binds to the receptor and competes with the wild-type cytokine/chemokine but does not trigger cytokine/chemokine signalling.

For example, the agent may be a dominant negative version of a cytokine receptor or chemokine receptor. A dominant negative version may, for example, be a mutated or truncated version of the cytokine/chemokine receptor which binds to the cytokine blocking its binding to the wild-type cytokine/chemokine receptor.

Alternatively, the agent may be an antibody or antibody fragment which blocks or otherwise modulates a cytokine or chemokine signalling pathway.

The activity modulator may be a chimeric cytokine receptor which comprises a cytokine receptor endodomain.

The activity modulator may comprise the exodomain from an immunoinhibitory cytokine, such as IL-4, fused to the endodomain from a cytokine such as IL-7 which enhances T-cell proliferation (Leen et al (2014) Mol. Ther. 22:1211-1220).

The activity modulator may be a chemokine such as CCL19. Chemokine (C—C motif) ligand 19 (CCL19) is a small cytokine belonging to the CC chemokine family that is also known as EBI1 ligand chemokine (ELC) and macrophage inflammatory protein-3-beta (MIP-3-beta). CCL19 elicits its effects on its target cells by binding to the chemokine receptor chemokine receptor CCR7. It attracts certain cells of the immune system, including dendritic cells and antigen-engaged B cells and CCR7+ central-memory T-Cells. The amino acid sequence for human CCL19 is available from UniProt (Accession number Q99731).

### 3.1 Chimeric Cytokine Receptors

Alternatively, the activity modulator may comprise a non-cytokine receptor exodomain. WO2017/029512 describes chimeric cytokine receptors (CCR) comprising: an exodomain which binds to a ligand selected from a tumour secreted factor, a chemokine and a cell-surface antigen; and a cytokine receptor endodomain.

The chimeric cytokine receptor may comprise two polypeptides:
(i) a first polypeptide which comprises:
   (a) a first antigen-binding domain which binds a first epitope of the ligand
   (b) a first chain of the cytokine receptor endodomain; and
(ii) a second polypeptide which comprises:
   (a) a second antigen-binding domain which binds a second epitope of the ligand (b) a second chain of the cytokine-receptor endodomain.

Alternatively the chimeric cytokine receptor which comprises two polypeptides:
(i) a first polypeptide which comprises:
   (a) a heavy chain variable domain (VH)
   (b) a first chain of the cytokine receptor endodomain; and
(ii) a second polypeptide which comprises:
   (a) a light chain variable domain (VL)
   (b) a second chain of the cytokine-receptor endodomain.

For example, the cytokine receptor endodomain may comprise:
(i) IL-2 receptor β-chain endodomain
(ii) IL-7 receptor α-chain endodomain;
(iii) IL-15 receptor α-chain endodomain; or
(iv) common γ-chain receptor endodomain.

The cytokine receptor endodomain may comprise (i), (ii) or (iii); and (iv).

The cytokine receptor endodomain may comprise the α-chain endodomain and the β-chain endodomain from granulocyte-macrophage colony-stimulating factor receptor (GMCSF-R)

The ligand may be a tumour secreted factor, for example a tumour secreted factor selected from: prostate-specific antigen (PSA), carcinoembryonic antigen (CEA), vascular endothelial growth factor (VEGF) and CA125.

The ligand may be a chemokine, for example a chemokine selected from chemokine selected from: CXCL12, CCL2, CCL4, CCL5 and CCL22.

The ligand may be a cell-surface molecule, such as a transmembrane protein. The ligand may be, for example, CD22.

### Constitutively Active Chimeric Cytokine Receptors

The activity modulator may be a constitutively active chimeric cytokine receptor. The activity modulator may comprise two chains which dimerise, either spontaneously or in the presence of an agent (a chemical inducer of dimerization or CID) bringing together two cytokine receptor endodomains.

The activity modulator may therefore comprise a dimerization domain; and a cytokine receptor endodomain.

Dimerisation may occur spontaneously, in which case the chimeric transmembrane protein will be constitutively active. Alternatively, dimerization may occur only in the presence of a chemical inducer of dimerization (CID) in which case the transmembrane protein only causes cytokine-type signalling in the presence of the CID.

Suitable dimerization domains and CIDs are described in WO2015/150771, the contents of which are hereby incorporated by reference.

For example, one dimerization domain may comprise the rapamycin binding domain of FK-binding protein 12 (FKBP12), the other may comprise the FKBP12-Rapamycin Binding (FRB) domain of mTOR; and the CID may be rapamycin or a derivative thereof.

One dimerization domain may comprise the FK506 (Tacrolimus) binding domain of FK-binding protein 12 (FKBP12) and the other dimerization domain may comprise the cyclosporin binding domain of cyclophilin A; and the CID may be an FK506/cyclosporin fusion or a derivative thereof.

One dimerization domain may comprise an oestrogen-binding domain (EBD) and the other dimerization domain may comprise a streptavidin binding domain; and the CID may be an estrone/biotin fusion protein or a derivative thereof.

One dimerization domain may comprise a glucocorticoid-binding domain (GBD) and the other dimerization domain may comprise a dihydrofolate reductase (DHFR) binding domain; and the CID may be a dexamethasone/methotrexate fusion protein or a derivative thereof.

One dimerization domain may comprise an 06-alkylguanine-DNA alkyltransferase (AGT) binding domain and the other dimerization domain may comprise a dihydrofolate reductase (DHFR) binding domain; and the CID may be an O6-benzylguanine derivative/methotrexate fusion protein or a derivative thereof.

One dimerization domain may comprise a retinoic acid receptor domain and the other dimerization domain may comprise an ecdysone receptor domain; and the CID may be RSL1 or a derivative thereof.

Where the dimerization domain spontaneously heterodimerizes, it may be based on the dimerization domain of an antibody. In particular it may comprise the dimerization portion of a heavy chain constant domain (CH) and a light chain constant domain (CL). The “dimerization portion” of a constant domain is the part of the sequence which forms the inter-chain disulphide bond.

The chimeric cytokine receptor may comprise the Fab portion of an antibody as exodomain. In this respect, the chimeric antigen may comprise two polypeptides:
(i) a first polypeptide which comprises:
   (a) a heavy chain constant domain (CH)
   (b) a first chain of the cytokine receptor endodomain; and
(ii) a second polypeptide which comprises:
   (a) a light chain constant domain (CL)
   (b) a second chain of the cytokine-receptor endodomain.

The cytokine receptor endodomain may comprise:
(i) IL-2 receptor β-chain endodomain
(ii) IL-7 receptor α-chain endodomain; or
(iii) IL-15 receptor α-chain endodomain; and/or
(iv) common γ-chain receptor endodomain.

The cytokine receptor endodomain may comprise the α-chain endodomain and the β-chain endodomain from granulocyte-macrophage colony-stimulating factor receptor (GMCSF-R)

A constitutively active CCR having an IL-2, IL-7 or GM-CSF receptor endodomain may have one of the following structures:
Fab_CCR_IL2: HuLightKappa-IL2RgTM-IL2RgEndo-2A-HuCH1-IL2bTM-IL2RbENDO Fab_CCR_IL7: HuLightKappa-IL2RgTM-IL2RgEndo-2A-HuCH1-IL7RaTM-IL7RaENDO

### Fab_CCR_GMCSF:

HuLightKappa-GMCSFRbTM-GMCSFRbEndo-2A-HuCH1-GMCSFRaTM-GMCSFRaENDO
   In which:
HuLightKappa is a human light kappa chain
IL2RgTM is a transmembrane domain from human IL2R common gamma chain
IL2RgEndo is an endodomain derived from human IL2R common gamma chain
2A is a sequence enabling the co-expression of the two polypeptides, which may be a self cleaving peptide such as a 2A peptide
HuCH1 is a human CH1
IL2bTM is a transmembrane domain from human IL-2R beta
IL2RbENDO is an endodomain from human IL2R beta
IL7RaTM is a transmembrane domain from human IL-7R alpha
IL7RaENDO is an endodomain from human IL-7R alpha
GMCSFRb™ is a transmembrane domain from Human GM-CSFR common beta chain
GMCSFRbEndo is an endodomain from GM-CSFR common beta chain
GMCSFRaTM is a transmembrane domain from Human GF-CSFR alpha
GMCSFRaENDO is an endodomain Derived from Human GM-CSFR alpha

The sequences for the components for making a constitutively active cytokine receptor as shown below as SEQ ID NO. 30 to 43.

SEQ ID No. 30
(Human Light Kappa Chain)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC
SEQ ID No. 31
(Human Hinge)
EPKSCDKTHTCPPCPKDPK
SEQ ID No. 32
(Human CH1)
STKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRV
SEQ ID No. 33
(Transmembrane domain from human IL2R common gamma
chain):
VVISVGSMGLIISLLCVYFWL
SEQ ID No. 34
(Transmembrane domain from human IL-2R beta)
IPWLGHLLVGLSGAFGFIILVYLLI
SEQ ID No. 36
(Transmembrane domain from human IL-7R alpha)
PILLTISILSFFSVALLVILACVLW
SEQ ID No. 37
(Transmembrane domain from Human GF-CSFR alpha)
NLGSVYIYVLLIVGTLVCGIVLGFLF
SEQ ID No. 38
Transmembrane domain from Human GM-CSFR common
beta chain)
VLALIVIFLTIAVLLAL
SEQ ID No. 39
(Endodomain from human IL2R common gamma chain)
ERTMPRIPTLKNLEDLVTEYHGNFSAWSGVSKGLAESLQPDYSERLCLVS
EIPPKGGALGEGPGASPCNQHSPYWAPPCYTLKPET
SEQ ID No. 40
(Endodomain from human IL-2R beta)
NCRNTGPWLKKVLKCNTPDPSKFFSQLSSEHGGDVQKWLSSPFPSSSFSP
GGLAPEISPLEVLERDKVTQLLLQQDKVPEPASLSSNHSLTSCFTNQGYF
FFHLPDALEIEACQVYFTYDPYSEEDPDEGVAGAPTGSSPQPLQPLSGED
DAYCTFPSRDDLLLFSPSLLGGPSPPSTAPGGSGAGEERMPPSLQERVPR
DWDPQPLGPPTPGVPDLVDFQPPPELVLREAGEEVPDAGPREGVSFPWSR
PPGQGEFRALNARLPLNTDAYLSLQELQGQDPTHLV
SEQ ID No. 41
(Endodomain from human IL-7R alpha)
KKRIKPIVWPSLPDHKKTLEHLCKKPRKNLNVSFNPESFLDCQIHRVDDI
QARDEVEGFLQDTFPQQLEESEKQRLGGDVQSPNCPSEDVVITPESFGRD
SSLTCLAGNVSACDAPILSSSRSLDCRESGKNGPHVYQDLLLSLGTTNST
LPPPFSLQSGILTLNPVAQGQPILTSLGSNQEEAYVTMSSFYQNEQ
SEQ ID No. 42
(Endodomain Derived from Human GM-CSFR alpha)
KRFLRIQRLFPPVPQIKDKLNDNHEVEDEIIWEEFTPEEGKGYREEVLTV
KEIT
SEQ ID No. 43
(Endodomain from GM-CSFR common beta chain)
RFCGIYGYRLRRKWEEKIPNPSKSHLFQNGSAELWPPGSMSAFTSGSPPH
QGPWGSRFPELEGVFPVGFGDSEVSPLTIEDPKHVCDPPSGPDTTPAASD
LPTEQPPSPQPGPPAASHTPEKQASSFDFNGPYLGPPHSRSLPDILGQPE
PPQEGGSQKSPPPGSLEYLCLPAGGQVQLVPLAQAMGPGQAVEVERRPSQ
GAAGSPSLESGGGPAPPALGPRVGGQDQKDSPVAIPMSSGDTEDPGVASG
YVSSADLVFTPNSGASSVSLVPSLGLPSDQTPSLCPGLASGPPGAPGPVK
SGFEGYVELPPIEGRSPRSPRNNPVPPEAKSPVLNPGERPADVSPTSPQP
EGLLVLQQVGDYCFLPGLGPGPLSLRSKPSSPGPGPEIKNLDQAFQVKKP
PGQAVPQVPVIQLFKALKQQDYLSLPPWEVNKPGEVC

The activity modulator may comprise a variant of one or more of SEQ ID NO: 30 to 43 having at least 80, 85, 90, 95, 98 or 99% sequence identity, provided that the variant sequence has the required properties. For example, a variant CH or CL sequence should retain the capacity to dimerise with a CL/CH containing-chain. A variant chain from a cytokine receptor endodomain should retain the capacity to trigger cytokine-mediated signalling when coupled with the reciprocal chain for that cytokine receptor.

### 3.3 JAK/STAT

Signal Transducer and Activator of Transcription (STAT) molecules are a family of transcription factors that are involved in cytokine-mediated signal transduction. STAT transcription factors are recruited to the cytoplasmic region of cell surface receptors and are activated via phosphorylation. Once activated, they dimerize to form an activated STAT molecule comprising a first polypeptide and a second polypeptide, and translocate into the cell nucleus where they influence gene expression. They play a role in regulating cell growth processes and cell differentiation. The JAK-STAT signalling pathway consists of three main components: (1) a receptor which penetrates the cell membrane (2) Janus kinase (JAK), which is bound to the receptor and (3) Signal Transducer and Activator of Transcription (STAT), which carries the signal into the nucleus and DNA (see FIG. 3).

It is possible to enhance engraftment and persistence of CAR-expressing cells by including in the cell a constitutively active or inducible active JAK or STAT molecule. International Patent Application No. PCT/GB2018/052583 describes various alternative arrangements for Constitutively active STAT molecules (FIG. 4 of International Patent Application No. PCT/GB2018/052583) and inducible STAT molecules (FIG. 5 of International Patent Application No. PCT/GB2018/052583).

As described in International Patent Application No. PCT/GB2018/052583, a constitutively active JAK molecule may be made by expressing two JAK polypeptides which spontaneously dimerise or are linked by a linker, as described below for constitutively active STAT molecules. Alternatively, constitutively active JAK may be expressed which comprises a gain-of-function mutation.

The activity modulator may be a constitutively active or an inducible Signal Transducer and Activator of Transcription (STAT) molecule.

The STAT molecule of the cell may a first STAT polypeptide comprising a first dimerizing domain (DD) and a second STAT polypeptide comprising a second DD, which binds to the first DD.

The first and second DDs of the STAT molecule of the cell may, for example, comprise leucine zipper domains; or a heavy chain constant region and a light chain constant region.

An inducible STAT molecule of the cell may be inducibly active in the presence of an agent which causes dimerization of the first DD and second DD of the STAT molecule, thereby inducing activation of the STAT molecule. For example, the first DD may comprise FRB, the second DD may comprise FKBP12 and the agent may be rapamycin.

Alternatively, the STAT molecule of the cell may be inducibly inactive in the presence of an agent which causes dissociation of the first DD and second DD of the STAT molecule, thereby inducing non-activation of the STAT molecule. The first DD may comprise TetRB and the second DD may comprise TiP and the agent may be tetracycline, doxycycline or minocycline.

A constitutively active STAT may comprise a Gain of Function (GOF) mutation or may comprise a first STAT polypeptide and a second STAT polypeptide linked by a linker sequence.

The cell may comprise a membrane-tethering molecule comprising a tethering domain and a first binding domain (BD), and a constitutively active STAT molecule which comprises a second BD which binds specifically to the first BD. Binding of the first and second BD may be disrupted by the presence of an agent, such that in the presence of the agent the constitutively active STAT molecule dissociates from the membrane-tethering molecule, so that the constitutively active STAT molecule is free to translocate to the nucleus.

The first and second DD of the STAT molecule of the cell; or the first BD of the membrane-tethering molecule of the cell and second BD of the STAT molecule of the cell may comprise a Tet Repressor Protein (TetR) and a Transcription Inducing Peptide (TiP), respectively; and the agent may be tetracycline, doxycycline or minocycline.

The cell may comprise a) a CAR and a constitutively active STAT molecule joined by a STAT release domain and b) a STAT release molecule which releases the constitutively active STAT molecule from the CAR at the STAT release domain only upon recognition of a target antigen specific to the CAR, such that upon release, the constitutively active STAT molecule is free to translocate to the nucleus.

The STAT release molecule may comprise a CAR targeting domain, for example which binds to a phosphorylated immunoreceptors tyrosine based activation motif (ITAM). For example, the CAR targeting domain may comprise one or more ZAP70 SH2 domains.

The STAT release domain of the cell of the present invention may comprise a protease cleavage site, and the STAT release molecule of the cell may comprise a protease domain, such that upon recognition of a target antigen of the CAR, the protease domain cleaves at the protease cleavage site, releasing the STAT molecule.

### 4. Adhesion Molecule

Cell adhesion molecules (CAMs) are proteins located on the cell surface involved in binding with other cells or with the extracellular matrix (ECM) in cell adhesion.

These proteins are typically transmembrane receptors and are composed of three domains: an intracellular domain that interacts with the cytoskeleton, a transmembrane domain, and an extracellular domain that interacts either with other CAMs of the same kind (homophilic binding) or with other CAMs or the extracellular matrix (heterophilic binding).

Most CAMs belong to four protein families: Ig (immunoglobulin) superfamily (IgSF CAMs), the integrins, the cadherins, and the selectins.

The activity modulator of the present invention may be or comprise an adhesion molecule which modulates CAR- or TCR-expressing cell activity.

### 5. Transcription Factor

The agent of the invention may be or comprise a transcription factor which modulates activity of the CAR- or TCR-expressing cell.

A transcription factor is a protein which controls the rate of transcription of genetic information from DNA to messenger RNA, by binding to a specific DNA sequence and regulate the expression of a gene which comprises or is adjacent to that sequence.

Transcription factors work by promoting (as an activator), or blocking (as a repressor) the recruitment of RNA polymerase.

Transcription factors contain at least one DNA-binding domain (DBD), which attaches to either an enhancer or promoter region of DNA. Depending on the transcription factor, the transcription of the adjacent gene is either up- or down-regulated. Transcription factors also contain a trans-activating domain (TAD), which has binding sites for other proteins such as transcription coregulators.

Transcription factors use a variety of mechanisms for the regulation of gene expression, including stabilizing or blocking the binding of RNA polymerase to DNA, or catalyzing the acetylation or deacetylation of histone proteins. The transcription factor may have histone acetyltransferase (HAT) activity, which acetylates histone proteins, weakening the association of DNA with histones and making the DNA more accessible to transcription, thereby up-regulating transcription. Alternatively the transcription factor may have histone deacetylase (HDAC) activity, which deacetylates histone proteins, strengthening the association of DNA with histones and making the DNA less accessible to transcription, thereby down-regulating transcription. Another mechanism by which they may function is by recruiting coactivator or corepressor proteins to the transcription factor DNA complex.

The transcription may be constitutively active or conditionally active, i.e. requiring activation.

The transcription factor may be naturally occurring or artificial.

### 5.1 Transcriptional Reprogramming

In order for a CAR-T cell to be effective, it is important that it persists and expands in vivo and resists overly rapid differentiation and exhaustion. CAR T-cell persistence and engraftment is related to the proportion of naïve, central memory and T-stem-cell memory T-cells administered.

WO2018/115865 describes a cell which co-expresses a chimeric antigen receptor (CAR) and a transcription factor. Expression of the transcription factor may prevent or reduce differentiation and/or exhaustion of the cell in vitro and/or in vivo. By co-expressing the CAR with a transcription factor in a cell, it is possible to prevent or reduce differentiation and/or exhaustion of the cell. This results in a greater proportion of naïve, central memory and stem-cell memory cells in the cell composition for immunotherapy, and more effective persistence and expansion of the cells in vivo.

The activity modulator of the present invention may be a transcription factor. The transcription factor may prevent or reduce differentiation and/or exhaustion of the cell.

The transcription factor may be an effector memory repressor, such as BLIMP-1

Alternatively, the transcription factor may be a central memory repressor such as BCL6 or Bach2.

The transcription factor may be or comprise Bach2 or a modified version of Bach2 which has reduced or removed capacity to be phosphorylated by ALK. For example, modified Bach2 may comprise a mutation at one or more of the following positions: Ser-535, Ser-509, Ser-520.

The transcription factor may be FOXO1, EOMES, Runx3 or CBF beta.

### 6. Modulating Tgfb Signalling

Engineered cells face hostile microenvironments which limit adoptive immunotherapy. One of the main inhibitory mechanisms within the tumour microenvironment is transforming growth factor beta (TGFβ). The TGFβ signalling pathway has a pivotal role in the regulatory signalling that controls a variety of cellular processes. TGFβ play also a central role in T cell homeostasis and control of cellular function. Particularly, TGFβ signalling is linked to an immuno-depressed state of the T-cells, with reduced proliferation and activation. TGFβ expression is associated with the immunosuppressive microenvironment of tumour.

A variety of cancerous tumour cells are known to produce TGFβ directly. In addition to the TGFβ production by cancerous cells, TGFβ can be produced by the wide variety of non-cancerous cells present at the tumour site such as tumour-associated T cells, natural killer (NK) cells, macrophages, epithelial cells and stromal cells.

The transforming growth factor beta receptors are a superfamily of serine/threonine kinase receptors. These receptors bind members of the TGFβ superfamily of growth factor and cytokine signalling proteins. There are five type II receptors (which are activatory receptors) and seven type I receptors (which are signalling propagating receptors).

Auxiliary co-receptors (also known as type III receptors) also exist. Each subfamily of the TGFβ superfamily of ligands binds to type I and type II receptors.

The three transforming growth factors have many activities. TGFβ1 and 2 are implicated in cancer, where they may stimulate the cancer stem cell, increase fibrosis/desmoplastic reactions and suppress immune recognition of the tumour.

TGFβ1, 2 and 3 signal via binding to receptors TβRII and then association to TβRI and in the case of TGFβ2 also to TβRIII. This leads to subsequent signalling through SMADs via TβRI.

TGFβs are typically secreted in the pre-pro-form. The “pre” is the N-terminal signal peptide which is cleaved off upon entry into the endoplasmic reticulum (ER). The “pro” is cleaved in the ER but remains covalently linked and forms a cage around the TGFβ called the Latency Associated Peptide (LAP). The cage opens in response to various proteases including thrombin and metalloproteases amongst others. The C-terminal region becomes the mature TGFβ molecule following its release from the pro-region by proteolytic cleavage. The mature TGFβ protein dimerizes to produce an active homodimer.

The TGFβ homodimer interacts with a LAP derived form the N-terminal region of the TGFβ gene product, forming a complex called Small Latent Complex (SLC). This complex remains in the cell until it is bound by another protein, an extracellular matrix (ECM) protein called Latent TGFβ binding protein (LTBP) which together forms a complex called the large latent complex (LLC). LLC is secreted to the ECM. TGFβ is released from this complex to a biologically active form by several classes of proteases including metalloproteases and thrombin.

The activity modulator of the present invention may modulate TGFβ signalling.

For example, the activity modulator may block or reduce TGFβ binding to TGFβ receptor; it may compete with TGFβ or TGFβR for binding to TGFβR or TGFβ; alternatively it may modulate the downstream TGFβ signalling for example via SMADs.

The activity modulator may be an agent such an antibody which binds TGFβ or TGFβ receptor.

Fresolimumab is a immunomodulatory antibody which blocks TGFβ1-3. Fresolimumab has been tested in metastatic melanoma and high-grade glioma. This showed some effectiveness in the enhancement of a tumour-specific immune response but failed to eradicate the tumour. Other antibodies which bind TGFβ include Lerdelimumab and Metelimumab.

Bedinger et al (2016) describes various human monoclonal antibodies that neutralize multiple TGFβ isoforms (MAbs 8 (2): 389-404)

Alternatively the activity modulator may be a recombinant Fc-fusion proteins containing the soluble ectodomain of either TβRII (TβRII-Fc) or the type III receptor, betaglycan. Soluble TβRII and soluble TβRIII (βglycan) function as decoy receptors preventing binding of TGF-β.

### 6.1 Dominant Negative TGFβ

The activity modulator may be a secreted factor which is capable of binding a transforming growth factor beta receptor (TβR) and disrupting its interaction with transforming growth factor beta (TGFβ).
The activity modulator may be a dominant negative TGFβ.
“Dominant negative TGFβ” or dnTGFβ as used herein means that the secreted factor
TGFβ acts antagonistically to the wild-type TGFβ.
The dominant negative TGFβ inhibits signalling induced by wild-type TGFβ and thus neutralise its biological effects.
The activity modulator may be a mutant TGFβ.

The mature protein of wild-type TGFβ2 is shown below as in SEQ ID NO: 44.

(SEQ ID NO: 44)
ALDAAYCFRNVQDNCCLRPLYIDFKRDLGWKWIHEPKGYNANFCAGACPY
LWSSDTQHSRVLSLYNTINPEASASPCCVSQDLEPLTILYYIGKTPKIEQ
LSNMIVKSCKCS.

A mutant TGFβ may comprise one or more mutations at amino acid residues W30, W32, L101, L51, Q67 and Y6 when the amino acid number is determined by alignment with SEQ ID NO: 44 and wherein:
amino acid residue 30 is mutated to N,R,K,D,Q,L,S,P,V,I,G,C,T,A or E; and/or
amino acid residue 32 is mutated to A; and/or
amino acid residue 101 is mutated to A, E; and/or
amino acid residue 51 is mutated to Q,W,Y,A; and/or
amino acid residue 67 is mutated to H, F, Y, W, Y; and/or
amino acid residue 6 is mutated to A or a variant thereof.

Alternatively the activity modulator may comprise a truncated TGFβ polypeptide such as monomeric TGFβ. Kim et al (2017) describe an engineered TGFβ monomer that functions as a dominant negative to block TGFβ signalling (J. Biol. Chem. doi: 10.1074/jbc.M116.768754).

Truncated TGFβ may lack the heel helix α3, a structural motif essential for binding the TGFβ type I receptor (TβRI) but dispensable for binding TβRII.

The amino acid sequence of a TGFβ monomer is set forth in SEQ ID NO: 45. SEQ ID NO: 45 comprises a signal peptide and a latency associated peptide (LAP).

(SEQ ID NO: 45)
MHYCVLSAFLILHLVTVALSLSTCSTLDMDQFMRKRIEAIRGQILSKLKL
TSPPEDYPEPEEVPPEVISIYNSTRDLLQEKASRRAAACERERSDEEYYA
KEVYKIDMPPFFPSENAIPPTFYRPYFRIVRFDVSAMEKNASNLVKAEFR
VFRLQNPKARVPEQRIELYQILKSKDLTSPTQRYIDSKVVKTRAEGEWLS
FDVTDAVHEWLHHKDRNLGFKISLHCPCCTFVPSNNYIIPNKSEELEARF
AGIDGTSTYTSGDQKTIKSTRKKNSGKTPHLLLMLLPSYRLESQQTNRRK
KRALDAAYCFRNVQDNCCLRPLYIDFRKDLGWKWIHEPKGYNANFCAGAC
PYRASKSPSCVSQDLEPLTIVYYVGRKPKVEQLSNMIVKSCKCS.

The activity modulator may have an amino acid sequence set forth in SEQ ID NO: 45 or a variant thereof. The variant TGFβ monomer may have at least 80%, 85%, 90%, 95%, 98% or 99% sequence identity to SEQ ID NO: 45, provided that the polypeptide provides a monomer which is capable of binding a transforming growth factor beta receptor (TβR) and disrupting its interaction with transforming growth factor beta (TGFβ).

### 6.2 Dominant Negative TGFβ Receptor

The active TGFβ receptor (TβR) is a hetero-tetramer, composed by two TGFβ receptor I (TβRI) and two TGFβ receptor II (TβRII). TGFβ1 is secreted in a latent form and is activated by multiple mechanisms. Once activated it forms a complex with the TβRII TβRI that phosphorylates and activates TβRI.

The activity modulator may be a dominant negative TGFβ receptor. A dominant negative TGFβ receptor may lack the kinase domain.

For example, the activity modulator may comprise or consist of the sequence shown as SEQ ID No. 46, which is a monomeric version of TGF receptor II

SEQ ID No. 46
(dn TGFβ RII)
TIPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKCI
MKEKKKPGETFFMCSCSSDECNDNIIFSEEYNTSNPDLLLVIFQVTGISL
LPPLGVAISVIIIFYCYRVNRQQKLSS

A dominant-negative TGF-βRII (dnTGF-βRII) has been reported to enhance PSMA targeted CAR-T cell proliferation, cytokine secretion, resistance to exhaustion, long-term in vivo persistence, and the induction of tumour eradication in aggressive human prostate cancer mouse models (Kloss et al (2018) Mol. Ther. 26:1855-1866).

### 6.3 SMADs

As mentioned above, the active TGFβ receptor (TβR) is a hetero-tetramer, composed by two TGFβ receptor I (TβRI) and two TGFβ receptor II (TβRII). Signaling is initiated when activated TGF-β binds to transforming growth factor-β receptor-2 (TβRII) with high affinity. This binding requires the participation of the transforming growth factor-β receptor-3 (TβRIII), also known as βglycan, which causes a conformational change in TβRII that facilitates ligand-receptor binding TGF-β receptor-1/ALK-5 (TβR1), a serine/threonine kinase, is then recruited to the TGF-β/TβRII complex and initiates signaling by phosphorylating SMAD2 and SMAD3, which belong to the receptor-regulated family of SMAD proteins. Phosphorylated SMAD2 and SMAD3 combine to form a heteromeric complex with SMAD4 that translocates to the cell nucleus to interact with various transcriptional factors that ultimately leads to the cellular response

SMAD proteins are intracellular transcription factors for conveying extracellular signal from membrane to the nucleus upon the activation of TGFβ. Three types of SMAD have been identified: a receptor-regulated SMAD s (R-SMAD) including SMAD 2 and SMAD 3 and a common-mediator SMAD (co-SMAD) including SMAD 4 only. Finally, an inhibitory SMAD s (I-SMAD) including SMAD 6 and SMAD 7.

SMAD proteins consist of two globular domains connected by a linker region. The main function of the SMAD N-terminal domain, or “Mad homology 1” (MH1) domain, is to bind DNA. The C-terminal domain, or MH2 domain, mediates protein-protein interaction with numerous regulator and effector proteins, including the TBR receptors, certain cytoplasmic anchor proteins, lineage-specific DNA-binding cofactors, and chromatin modifiers. In the presence of TGFβ, R-SMAD is phosphorylated by TGFβ receptor. This phosphorylation targets two serine residues in the SMAD C terminus sequence, pSer-X-pSer, creating an acidic tail that drives the formation of SMAD transcriptional complexes. Missense mutation of two conserved amino acid in the N terminal portion of the MH2 domain has been identified in patient with colorectal cancer. These two mutations cause the acquisition of a dominant negative behaviour that counteract the activity of the WT SMAD protein.

The activity modulator may be a SMAD signalling inhibitors such as Galunisertib which has been tested as a monotherapy or in combination with alkylating agents, Lomustine or temozolamide for glioblastoma and other combinations.

Alternatively the activity modulator may be a dominant negative version of the signal transduced SMAD 2 and SMAD 3 and SMAD4 expressing only the MH2 domain. The activity modulator may be: i) MH2, ii) MH2 truncated, missing the last 24 aa and iii) truncated SMAD 2_MH2-linker-SMAD 3_MH2. These dominant negatives compete with the wild type SMAD protein for the receptor-docking domains and for the binding with partner proteins thus reducing or blocking TGFβ signalling.

A dnSMAD may be selected from one or more of SMAD2, SMAD3 and or SMAD4. The dnSMAD lacks a functional MH1 domain.

The MH1 domain is a conserved MAD homology domain of at the N terminus of a SMAD protein. The MH1 domain is capable of DNA binding and negatively regulates the functions of the MH2 domain.

The MH2 domain is a conserved MAD homology domain of at the C terminus of a SMAD protein. The MH2 domain contain a central β-sandwich with a conserved loop-helix is capable of binding phospho-serine residues. The MH2 domain mediates protein: protein interactions with regulator and effector proteins, including the TBR receptors, cytoplasmic anchor proteins, lineage-specific DNA-binding cofactor and chromatin modifiers.

The activity modulator may comprise or consist essentially of or consists of a wild-type MH2 domain from SMAD2, SMAD3 and SMAD4. The amino acids sequences for these MH2 domains are shown below as SEQ ID Nos 47 to 49.

(SEQ ID NO: 47 - MH2 domain of SMAD2)
WCSIAYYELNQRVGETFHASQPSLTVDGFTDPSNSERFOLGLLSNVNRNA
TVEMTRRHIGRGVRLYYIGGEVFAECLSDSAIFVQSPNCNQRYGWHPATV
CKIPPGCNLKIFNNQEFAALLAQSVNQGFEAVYQLTRMCTIRMSFVKGWG
AEYRRQTVTSTPCWIELHLNGPLQWLDKVLTQMGSPSVRCSSMS
(SEQ ID NO: 48 - MH2 domain of SMAD3)
WCSISYYELNQRVGETFHASQPSMTVDGFTDPSNSERFCLGLLSNVNRNA
AVELTRRHIGRGVRLYYIGGEVFAECLSDSAIFVQSPNCNQRYGWHPATV
CKIPPGCNLKIFNNQEFAALLAQSVNQGFEAVYQLTRMCTIRMSFVKGWG
AEYRRQTVTSTPCWIELHLNGPLQWLDKVLTQMGSPSIRCSSVS
(SEQ ID NO: 49 - MH2 domain of SMAD4)
WCSIAYFEMDVQVGETFKVPSSCPIVTVDGYVDPSGGDRFCLGQLSNVHR
TEAIERARLHIGKGVQLECKGEGDVWVRCLSDHAVFVQSYYLDREAGRAP
GDAVHKIYPSAYIKVFDLRQCHRQMQQQAATAQAAAAAQAAAVAGNIPGP
GSVGGIAPAISLSAAAGIGVDDLRRLCILRMSFVKGWGPDYPRQSIKETP
CWIEIHLHRALQLLDEVLHTMPIADPQPLD

The activity modulator may comprise or consist essentially of a truncated version of one of the MH2 domains outlined above, with a deletion of up to 24 amino acids from the C terminus of the wild type MH2 domain.

The activity modulator may comprises a mutation in the MH2 domain which increases the binding affinity of the dnSMAD for a phosphorylated TGFβ receptor. The activity modulator may consists essentially of or consists of: a MH2 domain of a SMAD4 polypeptide comprising a mutation R497H, K507Q and/or R515G where the amino acid numbering corresponds to the numbering set forth in SEQ ID NO: 49; an MH2 domain of SMAD3 comprising a mutation K378R and/or K314R where the amino acid numbering corresponds to the numbering set forth in SEQ ID NO:48.

The activity modulator may be a chimeric dnSMAD which comprises at least two dnSMAD polypeptides as defined above. The dnSMAD polypeptides of the chimeric dnSMAD may be connected by a linker domain.

The amino acid sequence of a chimeric dnSMAD comprising a dnSMAD2 polypeptide and a dnSMAD3 polypeptide is shown below as SEQ ID No. 50.

(SEQ ID NO: 50)
WCSIAYYELNQRVGETFHASQPSLTVDGFTDPSNSERFCLGLLSNVNRNA
TVEMTRRHIGRGVRLYYIGGEVFAECLSDSAIFVQSPNCNQRYGWHPATV
CKIPPGCNLKIFNNQEFAALLAQSVNQGFEAVYQLTRMCTIRMSFVKGWG
AEYRRQTVTSTPCWIELHLNGPLQWLDKVLTQMLEYSGGGSGGGSLEWCS
ISYYELNQRVGETFHASQPSMTVDGFTDPSNSERFCLGLLSNVNRNAAVE
LTRRHIGRGVRLYYIGGEVFAECLSDSAIFVQSPNCNQRYGWHPATVCKI
PPGCNLKIFNNQEFAALLAQSVNQGFEAVYQLTRMCTIRMSFVKGWGAEY
RRQTVTSTPCWIELHLNGPLQWLDKVLTQM

A dnSMAD or chimeric dnSMAD may comprise a sequence shown as: SEQ ID NO: 47 to 50; or a variant having at least 80% (preferably at least 85%, at least 90%, at least 95%, at least 97%, or at least 99%) sequence identity to SEQ ID NO: 47 to 50 provided that the variant sequence retains the capacity to compete with the wild type SMAD protein for the receptor-docking domains and for the binding with partner proteins and reduces or blocks TGFβ signalling.

### 7. Metabolic Enzymes

The activity modulator may be a metabolic enzyme such as AMP-activated protein kinase (AMPK) or Isocitrate dehydrogenase (IDH)

AMPK plays a role in cellular energy homeostasis, largely to activate glucose and fatty acid uptake and oxidation when cellular energy is low.

AMPK is a heterotrimeric protein complex formed by α, β, and γ subunits. Each of these three subunits takes on a specific role in both the stability and activity of AMPK. Specifically, the γ subunit includes four particular Cystathionine beta synthase (CBS) domains giving AMPK its ability to sensitively detect shifts in the AMP:ATP ratio. The four CBS domains create two binding sites for AMP commonly referred to as Bateman domains. Binding of one AMP to a Bateman domain cooperatively increases the binding affinity of the second AMP to the other Bateman domain. As AMP binds both Bateman domains the γ subunit undergoes a conformational change which exposes the catalytic domain found on the a subunit. It is in this catalytic domain where AMPK becomes activated when phosphorylation takes place at threonine-172 by an upstream AMPK kinase (AMPKK). The α, β, and γ subunits can also be found in different isoforms: the γ subunit can exist as either the γ1, γ2 or γ3 isoform; the β subunit can exist as either the β1 or B2 isoform; and the a subunit can exist as either the α1 or α2 isoform.

The following human genes encode AMPK subunits:
α-PRKAA1, PRKAA2
β-PRKAB1, PRKAB2
γ-PRKAG1, PRKAG2, PRKAG3

The activity modulator may comprise one or more AMPK subunits. The activity modulator may comprise α, β, and γ subunits from AMPK.

IDH catalyzes the oxidative decarboxylation of isocitrate, producing alpha-ketoglutarate (α-ketoglutarate) and CO₂. This is a two-step process, which involves oxidation of isocitrate (a secondary alcohol) to oxalosuccinate (a ketone), followed by the decarboxylation of the carboxyl group beta to the ketone, forming alpha-ketoglutarate. In humans, IDH exists in three isoforms: IDH3 catalyzes the third step of the citric acid cycle while converting NAD+ to NADH in the mitochondria. The isoforms IDH1 and IDH2 catalyze the same reaction outside the context of the citric acid cycle and use NADP+ as a cofactor instead of NAD+. They localize to the cytosol as well as the mitochondrion and peroxisome.

The activity modulator may IHD1, IHD2 or IHD3. The amino acid sequences for human IDH1, 2 and 3 are on the NCBI database with the following accession numbers: CAG38738.1 (IDH1); NP_002159.2 (IDH2, isoform 1); NP_001276839.1 (IDH2, isoform 2); NP_001277043.1 (IDH2, isoform 3); NP_689996.4 (IDH3, isoform 1); NP_001274178.1 (IDH3, isoform 2); NP_001339753.1 (IDH3, isoform 3).

### 8. Co-Stimulatory Signals

The activity modulator of the present invention may provide co-stimulatory signal to the T-cell.

For example, the activity modulatory may be a TNF receptor, a chimeric TNF receptor or a TNF receptor ligand.

TNF-family co-stimulatory molecules provide survival and expansion signals for T-cells during their ontogeny. These TNF receptors (TNFRs) signal via TNF receptor associated factor (TRAF) second messengers.

TNFRSF9 (4-1BB), TNFRSF4 (OX40), TNFRSF5 (CD40) and TNFRSF14 (GITR) transmit survival signals to T-cells. TNFRSF7 (CD27) and TNFRSF14 (HVEM) are expressed by naïve T-cells. The expression of OX40 and 4-1BB is induced in response to antigen stimulation, these TNFRs have been proposed to be markers of effector T cells. Although CD27 and GITR can be constitutively expressed by conventional T cells, their expression is also strongly upregulated following T-cell activation, possibly in parallel with the upregulation of OX40 and 4-1BB expression.

The induction or upregulation of OX40, 4-1BB and GITR expression occurs within 24 hours following the recognition of antigen by and activation of naive T cells, and much more rapidly by memory T cells; the expression of these receptors can last for several hours or even days.

The TNF receptor TNFRSF35/Death receptor 3 (D3R) is activated by TL1A which is upregulated by inflamed tissue transiently and this interaction appears to be important for the late stage of T-cell activity after an established immune response.

CD40 is not expressed by T-cells, but CD40L is and CD40/CD40L is particularly important for B-cell differentiation and expansion.

TNFRSF11A (RANK) is not expressed by T-cells, but the RANK/RANK-L pathway is important to immune development as well as being a key pathway for osteoclast activity and is active during bone metastasis.

TNFRSF12A (Fn14) is not expressed by T-cells, but is expressed along with its ligand TWEAK in damaged or inflamed tissues and most cancers.

### 8.1 Chimeric TNF Receptors

International Patent Application No. PCT/GB2018/053629 describes chimeric TNF receptors which comprise (a) a binding domain which is capable of binding a TNFR ligand; and (b) a TNFR signalling domain.

The presence of a chimeric TNFR enables the tight temporal and/or spatial control of TNFR signalling to be decoupled in order to provide improved survival signals for engineered cells, for example CAR T cells. The chimeric TNFR may compensate for the lack of a complete physiological immune response in a tumour microenvironment. The chimeric TNFR may be constructed such that the antigen-binding domain is engaged, and thus a required co-stimulatory signal induced, in the tumour microenvironment.

The antigen-binding domain of the chimeric TNFR may comprise the ligand binding domain of a TNFR. For example, the antigen-binding domain may comprise the ligand binding domain of D3R, HVEM, CD27, CD40, RANK or Fn14.

The signalling domain of the chimeric TNFR may be an activating signalling domain, such as one which is capable of signalling via TNFR-associated factors (TRAFs). For example, the activating signalling domain may comprise the signalling portion of the 4-1BB, OX40, or GITR endodomain.

The activity modulator may be a HVEM-41BB chimera, a CD27-41BB chimera, a RANK-41BB chimera or an Fn14-41BB chimera. Examples of suitable amino acid sequences for these chimeric TNF receptors are shown below as SEQ ID No. 51 to 54, in which the ectodomain is shown in normal text, the transmembrane domain in bold and the 41BB endodomain in italics.

SEQ ID No. 51
(HVEM-41BB)
MEPPGDWGPPPWRSTPRTDVLRLVLYLTFLGAPCYAPALPSCKEDEYPVG
SECCPKCSPGYRVKEACGELTGTVCEPCPPGTYIAHLNGLSKCLQCQMCD
PAMGLRASRNCSRTENAVCGCSPGHFCIVQDGDHCAACRAYATSSPGQRV
QKGGTESQDTLCQNCPPGTFSPNGTLEECQHQTKCSWLVTKAGAGTSSSH
WVIISFFLALTSTALLFLLFFLTLRFSVVKRGRKKLLYIFKQPFMRPVQT
TQEEDGCSCRFPEEEEGGCEL
SEQ ID No. 52
(CD27-41BB)
MARPHPWWLCVLGTLVGLSATPAPKSCPERHYWAQGKLCCQMCEPGTFLV
KDCDQHRKAAQCDPCIPGVSFSPDHHTRPHCESCRHCNSGLLVRNCTITA
NAECACRNGWQCRDKECTECDPLPNPSLTARSSQALSPHPQPTHLPYVSE
MLEARTAGHMQTLADFRQLPARTLSTHWPPQRSLCSSDFIRIISFFLALT
STALLFLLFFLTLRFSVVKRGRKKLLYIFKQPFMRPVQTTQEEDGCSCRF
PEEEEGGCEL
SEQ ID No. 53
(RANK-41BB)
MAPRARRRRPLFALLLLCALLARLQVALQIAPPCTSEKHYEHLGRCCNKC
EPGKYMSSKCTTTSDSVCLPCGPDEYLDSWNEEDKCLLHKVCDTGKALVA
VVAGNSTTPRRCACTAGYHWSQDCECCRRNTECAPGLGAQHPLQLNKDTV
CKPCLAGYFSDAFSSTDKCRPWTNCTFLGKRVEHHGTEKSDAVCSSSLPA
RKPPNEPHVYLPIISFFLALTSTALLFLLFFLTLRFSVVKRGRKKLLYIF
KQPFMRPVQTTQEEDGCSCRFPEEEEGGCEL
SEQ ID No. 54
(Fn14-41BB)
MARGSLRRLLRLLVLGLWLALLRSVAGEQAPGTAPCSRGSSWSADLDKCM
DCASCRARPHSDFCLGCAAAPPAPFRLLWPIISFFLALTSTALLFLLFFL
TLRFSVVKRGRKKLLYIFKQPFMRPVQTTQEEDGCSCRFPEEEEGGCEL

### 8.2 TNF Receptor Ligands

The TNF-related cytokines (TNF family ligands) are type II transmembrane proteins (intracellular N-terminus) with a short cytoplasmic tail (15 to 25 residues in length) and a larger extracellular region (˜50 amino acids) containing the signature TNF homology domain where the receptor binding sites are located.

A summary of TNFRs and their ligands is provided in Table 3.

TABLE 3

| Protein | | | |
|---|---|---|---|
| (member #) | Synonyms | Gene | Ligand(s) |
| Tumor necrosis | CD120a | TNFRSF1A | TNF-alpha |
| factor receptor 1 | | | (cachectin) |
| Tumor necrosis | CD120b | TNFRSF1B | TNF-alpha |
| factor receptor 2 | | | (cachectin) |
| Lymphotoxin beta | CD18 | LTBR | Lymphotoxin |
| receptor | | | beta (TNF-C) |
| OX40 | CD134 | TNFRSF4 | OX40L |
| CD40 | Bp50 | CD40 | CD154 |
| Fas receptor | Apo-1, CD95 | FAS | FasL |
| Decoy receptor 3 | TR6, M68 | TNFRSF6B | FasL, LIGHT, |
| | | | TL1A |
| CD27 | S152, Tp55 | CD27 | CD70, Siva |
| CD30 | Ki-1 | TNFRSF8 | CD153 |
| 4-1BB | CD137 | TNFRSF9 | 4-1BB ligand |
| Death receptor 4 | TRAILR1, Apo-2, | TNFRSF10A | TRAIL |
| | CD261 | | |
| Death receptor 5 | TRAILR2, CD262 | TNFRSF10B | TRAIL |
| Decoy receptor 1 | TRAILR3, LIT, | TNFRSF10C | TRAIL |
| | TRID, CD263 | | |
| Decoy receptor 2 | TRAILR4, | TNFRSF10D | TRAIL |
| | TRUNDD, CD264 | | |
| RANK | CD265 | TNFRSF11A | RANKL |
| Osteoprotegerin | OCIF, TR1 | TNFRSF11B | |
| TWEAK receptor | Fn14, CD266 | TNFRSF12A | TWEAK |
| TACI | IGAD2, CD267 | TNFRSF13B | APRIL, BAFF, |
| | | | CAMLG |
| BAFF receptor | CD268 | TNFRSF13C | BAFF |
| Herpesvirus entry | ATAR, TR2, | TNFRSF14 | LIGHT |
| mediator | CD270 | | |
| Nerve growth | p75NTR, CD271 | NGFR | NGF, BDNF, |
| factor receptor | | | NT-3, NT-4 |
| B-cell maturation | TNFRSF13A, | TNFRSF17 | BAFF |
| antigen | CD269 | | |
| Glucocorticoid- | AITR, CD357 | TNFRSF18 | GITR ligand |
| induced TNFR- | | | |
| related | | | |
| TROY | TAJ, TRADE | TNFRSF19 | unknown |
| Death receptor 6 | CD358 | TNFRSF21 | |
| Death receptor 3 | Apo-3, TRAMP, | TNFRSF25 | TL1A |
| | LARD, WS-1 | | |
| Ectodysplasin A2 | XEDAR | EDA2R | EDA-A2 |
| receptor | | | |

The activity modulator may be or comprise a TNF receptor ligand such as CD40L (CD154), OX40L (CD134) or 41BBL. The amino acid sequences for these proteins are shown below as SEQ ID No. 55 to 57

SEQ ID No. 55
(CD40L)
MIETYNQTSPRSAATGLPISMKIFMYLLTVFLITQMIGSALFAVYLHRRL
DKIEDERNLHEDFVFMKTIQRCNTGERSLSLLNCEEIKSQFEGFVKDIML
NKEETKKENSFEMQKGDQNPQIAAHVISEASSKTTSVLQWAEKGYYTMSN
NLVTLENGKQLTVKRQGLYYIYAQVTFCSNREASSQAPFIASLCLKSPGR
FERILLRAANTHSSAKPCGQQSIHLGGVFELQPGASVFVNVTDPSQVSHG
TGFTSFGLLKL
SEQ ID NO. 56
(OX40L)
MERVQPLEENVGNAARPRFERNKLLLVASVIQGLGLLLCFTYICLHFSAL
QVSHRYPRIQSIKVQFTEYKKEKGFILTSQKEDEIMKVQNNSVIINCDGF
YLISLKGYFSQEVNISLHYQKDEEPLFQLKKVRSVNSLMVASLTYKDKVY
LNVTTDNTSLDDFHVNGGELILIHQNPGEFCVL
SEQ ID NO. 57
(41BBL)
MEYASDASLDPEAPWPPAPRARACRVLPWALVAGLLLLLLLAAACAVFLA
CPWAVSGARASPGSAASPRLREGPELSPDDPAGLLDLRQGMFAQLVAQNV
LLIDGPLSWYSDPGLAGVSLTGGLSYKEDTKELVVAKAGVYYVFFQLELR
RVVAGEGSGSVSLALHLQPLRSAAGAAALALTVDLPPASSEARNSAFGFQ
GRLLHLSAGQRLGVHLHTEARARHAWQLTQGATVLGLFRVTPEIPAGLPS
PRSE

### Chimeric RTK

Receptor Protein-Tyrosine Kinase (RPTKs) constitute a family of intracellular signal regulators that mediate embryonic development, cell growth, metabolism, and immune function. RPTKs are often dysregulated in cancer, driving proliferation.

The proliferative capacity of CAR T-cells is inconsistent between different binders and structures, and the characteristics bestowing maximum proliferative incentive are elusive. CAR T-cells are exposed to the hostile tumour microenvironment, and incorporation of RPTK in a dimer format can provide a proliferative advantage to the CAR T-cells. Expression of an RTK which is capable of signalling in the absence of cognate ligand breaks the tumour immune-depriving milieu in order to improve the CAR T-cell survival signal.

RTKs signal through ligand-induced dimerization/oligomerisation, which leads to auto-phosphorylation of tyrosine residues in the kinase domain activation loop of their cytoplasmic tail. Ligand-mediated oligomerisation of the RTK leads to a two-step activation, the increase in catalytic activity and the creation of docking sites for downstream signalling proteins.

Typically RTKs homodimerize in order to signal.

The RTK auto-phosphorylation may occur in cis or in trans. The phosphorylated tyrosine residues constitute docking sites for numerous SH2-containing signalling molecules. Generally, all RTKs signal through common downstream signal protein such as: PI3 kinase, Ras-Raf-MAPK, JNK, and PLCγ. The signalling is mediated by the JAK-STAT pathway.

The activity modulator may be a receptor tyrosine kinase (RTK) which is capable of signalling in the absence of cognate ligand. Such RTK are described in GB patent application No. 1803079.1.

The RTK may be over-expressed and/or comprise a mutation such that it is capable of signaling in the absence of cognate ligand.

The RTK may be a chimeric RTK. The chimeric RTK may comprise an ectodomain or an endodomain which mediates dimerization or oligomerization of the chimeric RTK.

The domain which mediates dimerization or oligomerization of the chimeric RTK may comprise a disulphide bond, for example it may be or comprise a hinge domain.

Alternatively, the domain which mediates dimerization or oligomerization of the chimeric RTK may comprise a chemically operable dimerization or oligomerization domain. Dimerization or oligomerization of the chimeric RTK may inducible by an agent.

A summary of human RTK subfamilies and RTKs is shown in Table 4.

TABLE 4

| RTK | | RTK | | RTK | |
|---|---|---|---|---|---|
| Family | RTK | Family | RTK | Family | RTK |
| Epidermal | EGFR | Fibro- | FGFR-1 | Related | RYK |
| growth | | blast | | to | |
| factor | | growth | | receptor | |
| receptor | | factor | | tyrosine | |
| (EGFR) | | receptor | | kinase | |
| | | (FGFR) | | (RYK) | |
| | ERBB2 | | FGFR-2 | Discoidin | DDR1 |
| | ERBB3 | | FGFR-3 | domain | DDR2 |
| | | | | receptor | |
| | | | | family | |
| | | | | (DDR) | |
| | ERBB4 | | FGFR-4 | RET | RET |
| Insulin | INSR | KLG/CCK | CCK4 | Leukocyte | LTK |
| Receptor | IGF-1R | Erythro- | EPHA1 | receptor | ALK |
| | | poietin- | | tyrosine | |
| | | producing | | kinase | |
| | | human | | (LTK) | |
| | IRR | hepato- | EPHA2 | Muscle- | MUSK |
| | | cellular | | Specific | |
| | | receptors | | Kinase | |
| | | (EPHR) | | (MUSK) | |
| Platelet | PDGFR-α | | EPHA3 | NGFR | TRKA |
| derived | PDGFR-β | | EPHA4 | | TRKB |
| growth | CSF-1R | | EPHA5 | | TRKC |
| factor | KIT/SCFR | | EPHA6 | Hepatocyte | MET |
| receptor | KLK2/FLT3 | | EPHA7 | growth | RON |
| (PDGFR) | | | | factor | |
| | | | | receptor | |
| | | | | (HGFR) | |
| Vascular | VEGFR1 | | EPHA8 | LMR | AATYK |
| endothe- | VEGFR2 | | EPHB1 | | AATYK2 |
| lial growth | VEGFR3 | | EPHB2 | | AATYK3 |
| factor | | | | | |
| receptor | | | | | |
| (VEGFR) | | | | | |
| TIE | TIE | | EPHB3 | Tyrosine- | AXL |
| | TEK | | EPHB4 | protein | MER |
| Receptor | ROR1 | | EPHB5 | kinase | TYRO3 |
| tyrosine | | | | receptor | |
| kinase-like | | | | (AXL) | |
| orphan | ROR2 | | EPHB6 | Unknown | RTK106 |
| receptors | | | | | |
| (ROR) | | | | | |
| ROS | ROS | | | | |

Illustrative UniProt Accession Numbers and associated amino acid sequences of the human RTKs shown in Table 2 are provided in Table 5.

TABLE 5

| RTK | UniProt Accession Number |
|---|---|
| EGFR | P00533 |
| ERBB2 | P04626 |
| ERBB3 | P21860 |
| ERBB4 | Q15303 |
| INSR | P06213 |
| IGF-1R | P08069 |
| IRR | P14616 |
| PDGFR-α | P16234 |
| PDGFR-β | P09619 |
| CSF-1R | P07333 |
| KIT/SCFR | P10721 |
| KLK2/FLT3 | P20151 |
| VEGFR1 | P17948 |
| VEGFR2 | P35968 |
| VEGFR3 | P35916 |
| TIE | P35590 |
| TEK | Q02763 |
| ROR1 | Q01973 |
| ROR2 | Q01974 |
| ROS | P08922 |
| FGFR-1 | P11362 |
| FGFR-2 | P21802 |
| FGFR-3 | P22607 |
| FGFR-4 | P22455 |
| CCK4 | Q13308 |
| EPHA1 | P21709 |
| EPHA2 | P29317 |
| EPHA3 | P29320 |
| EPHA4 | P54764 |
| EPHA5 | P54756 |
| EPHA6 | Q9UF33 |
| EPHA7 | Q15375 |
| EPHA8 | P29322 |
| EPHB1 | P54762 |
| EPHB2 | P29323 |
| EPHB3 | P54753 |
| EPHB4 | P54760 |
| EPHB5 | |
| EPHB6 | O15197 |
| RYK | P34925 |
| DDR1 | Q08345 |
| DDR2 | Q16832 |
| RET | P07949 |
| LTK | P29376 |
| ALK | Q9UM73 |
| MUSK | O15146 |
| TRKA | P04629 |
| TRKB | Q16620 |
| TRKC | Q16288 |
| MET | P08581 |
| RON | Q04912 |
| AATYK | Q6ZMQ8 |
| AATYK2 | Q8IWU2 |
| AATYK3 | Q5XJV6 |
| AXL | P30530 |
| MER | Q12866 |
| TYRO3 | Q06418 |
| RTK106 | |

### Modulating Target Cell Activity

The activity modulator may modulate the activity of the target cell, for example, the tumour cell.

For example, the agent may be a toxin, such as a toxin which is toxic to tumour cells. For example, the agent may be diphtheria toxin, pseudomonas toxin or shigella toxin.

Alternatively the activity modulator may be a pro-drug or a pro-drug activating compound. The activity modulator may be a pro-drug activating enzyme.

Prodrugs are widely used in the targeted delivery of cytotoxic compounds to cancer cells. Prodrugs are inactive or less active derivatives of drug molecules which undergo enzymatic or chemical transformation to regenerate the active forms.

In targeted cancer therapy, conventional chemotherapeutic agents, which lack intrinsic target specificity, are rationally modified to focus and redirect their cytotoxicity to tumor cells. The usefulness of many conventional, nonspecific chemotherapeutic agents, such as doxorubicin, paclitaxel, camptothecin, cisplatin, and their derivatives have been significantly extended by modification into prodrugs, particularly those harboring cell-targeting moieties.

The activity modulator may be an enzyme which activates a specific inactive substrate (prodrug), which is administered separately, to a cytotoxic product. The activity modulator may be or comprise cytosine deaminase (CD) which converts the prodrug 5-fluorocytosine (5-FC) to 5-fluorouracil (5-FU), whose downstream antimetabolites lead to a so-called “thymineless death”. Alternatively the activity modulator may be or comprise a cytosine deaminase/uracil phospho-ribosyltransferase fusion (CD/UPRT; encoded by the Fcy::Fur gene) which has also been used to generate the 5-FU-based antimetabolites. Other antimetabolite prodrugs include the nucleoside analogs such as acyclovir and ganciclovir, which are activated to their active triphosphate using recombinant thymidylate kinase, as well as 6-methyl-2′-deoxyriboside and 2-fluoro-2′-deoxyadenosine, which are converted by purine nucleoside phosphorylase to 6′-methylpurine and 2-fluoroadenine, respectively. Human deoxycytidine kinase (DCK) and thymidylate kinase (tmpk) are capable of mono-phosphorylating a range of (non-physiologic) prodrugs such as gemcitabine (dFdC), bromovinyl-deoxyuridine (BVdU), cytarabine (AraC), and 3′-azido-3′-deoxythymidine (AZT) monophosphate. A chimeric fusion of DCK with uridine monophosphate kinase (DCK::UMK) has also been developed to directly activate gemcitabine to its cytotoxic diphosphate metabolite (dFdCDP) in pancreatic carcinoma. There are also “designer” prodrugs in which a chemotherapeutic agent is derivatized to a substrate for a specific activating enzyme. Examples include phenoxyacetamide conjugates of doxorubicin and melphalan that are hydrolyzed by penicillin-V amidase, a dipiperidinyl conjugate of etoposide (VP-16) that is hydrolyzed by a recombinant carboxylesterase, and a cephalosporin conjugate of 5-FU designed for hydrolysis by β-lactamase.

The activity modulator may be an enzyme capable of cleaving a prodrug-congugate for example, a pro-drug conjugate comprising a toxin. Like prodrug conjugates, many targeted toxins consist of a targeting moiety (e.g., an antibody, in the case of immunotoxins), a cleavable linker, and a drug (cytotoxic enzyme). Moxetumomab pasudotox, consists of a truncated exotoxin A from Pseudomonas aeruginosa in which the native receptor-binding domain (located in the N-terminal 250 residues) has been replaced with a single-chain variable fragment targeting the cell-surface CD22 antigen. Cytotoxic activity is conferred entirely by the C-terminal segment (residues 405 to 613, termed PE3). As is, this conjugate is an inactive toxin: cytotoxic activation requires cleavage by the protease furin during endocytosis between residues 279 and 280. Thus, moxetumomab pasudotox is functionally a targeted prodrug conjugate in which residues 251 to 364 from exotoxin A (domain II) serves as a linker whose cleavage releases the cytotoxic PE3.

### 1. Biosynthetic CAR T Cell

The activity modulator may be an enzyme which is capable of synthesising a small molecule when expressed or expressed in combination in a cell.

International patent application No. PCT/GB2018/053262 describes engineered cells which encode a transgenic synthetic biology pathway that enables the engineered cell to produce a small molecule, in particular a therapeutic small molecule. The engineered cell may comprises; (i) a chimeric antigen receptor (CAR) or a transgenic T-cell receptor (TCR); and (ii) one or more engineered polynucleotides which encode one or more enzymes which are capable of synthesising a therapeutic small molecule when expressed in combination in the cell.

There may be, for example one, two, three four or five enzymes. The one or more enzymes may be encoded in one or more open reading frames. The one or more enzymes may be encoded in a single open reading frame. Suitably, each enzyme may be separated by a cleavage site. The cleavage site may be a self-cleavage site, such as a sequence encoding a FMD-2A like peptide.

The therapeutic small molecule may, for example be a cytotoxic molecule; a cytostatic molecule; an agent which is capable of inducing differentiation of the tumour; or a proinflammatory molecule. In particular, the small molecule may be violacein or a derivative thereof, or geraniol.

### Target Cell Microenvironment Modulating Agent

The activity modulator may be an agent which modulates the environment of the target cell, for example, the tumour cell.

For example, the agent may be a cytokine such as IL-7 or IL-12 or a chemokine such as CCL19 as discussed above. Alternatively, the agent may affect the expression or activity of a cytokine or chemokine as discussed above.

### 1. CAR-T Cell Secreting Enzymes

The immune microenvironment contains small molecule metabolites and nutrients which can alter the balance between the tumour survival and/or progression and an immune response. Modulation of the microenvironment may alter the balance in favour of the immune response and/or can improve the activity or efficacy of adaptive immune therapy, such as the efficacy of engineered cells expressing a CAR or transgenic TCR.

An activity modulatory in connection with the present invention may modulate the level of one or more metabolites or nutrients in the tumour microenvironment, skewing the balance in favour of immune cells such as T-cells involved in an immune response and/or away from tumour cells.

For example, the activity modulator be one or more enzymes which, when secreted or expressed at the cell surface, causes depletion of a molecule extracellular to the engineered cell which is:
(i) required by a tumour cell for survival, proliferation, metastasis or chemoresistance, and/or
(ii) detrimental to the survival, proliferation or activity of the engineered cell.

United Kingdom application No. 1820443.8 describes engineered cells which secrete such enzymes or express them at the cell surface.

The enzyme(s) may cause the depletion of an amino acid or amino acid metabolite, a nucleobase (such as a nucleoside or nucleotide) or a lipid.

Where the activity modulator causes the depletion of a nutrient or metabolite which is needed for tumour cell growth or survival, the immune cell (e.g. CAR-T cell) may be engineered to survive in the absence of the molecule in the extracellular environment. For example, the cell may be engineered to synthesise the molecule or a precursor thereof intracellularly.

### Cell Composition

The present invention also provides a cell composition made by the method of the present invention.

The invention provides cell composition made by transduction of cells with a plurality of viral vectors such that the composition comprises a mixture of untransduced cells, singly transduced cells and combinatorially transduced cells.

At least one vector in the mixture of viral vectors used in the method of the present invention comprises a nucleic acid sequence encoding a CAR. The cell composition may therefore comprise a mixture of singly and combinatorially transduced CAR-expressing cells.

“Combinatorially transduced” means that the cell is transduced with at least two viral vectors. For example, if cells are transduced with two vectors, one comprising transgene A and one comprising transgene B, the transduced cells will be a mixture of cells expressing A alone; B alone; and cell expressing both A and B. In this situation, cells expressing A and B are combinatorially transduced.

For cells transduced with three vectors each comprising a transgene, the resulting transduced cells will be a mixture of: A alone; B alone; C alone; A and B; A and C; B and C; and cells expressing A, B and C. In this situation, the three sub-populations, expressing A and B; A and C; B and C; and cells expressing A, B and C are combinatorially transduced.

The cell composition comprises a plurality of sub-populations derived by transduction with different vector combinations in the mixture of viral vectors.

The cell composition may comprise cytolytic immune cells such as a T cells and/or or NK cells.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

Natural Killer cells (or NK cells) form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The cells of the invention may be any of the cell types mentioned above.

The cells to be transduced with a method of the invention may be derived from a blood sample, for example from a leukapheresate. The cells may be or comprise peripheral blood mononuclear cells (PBMCs).

Cells may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, cells may be derived from ex vivo differentiation of inducible progenitor cells or embryonic progenitor cells to, for example, T or NK cells. Alternatively, an immortalized T-cell line which retains its lytic function and could act as a therapeutic may be used.

The cells may be activated and/or expanded prior to being transduced with nucleic acid encoding the molecules providing the chimeric polypeptide according to the first aspect of the invention, for example by treatment with an anti-CD3 monoclonal antibody.

After transduction, the cells may then by purified, for example, selected on the basis of expression of the CAR. It may be desirable to select cells on the basis of CAR expression when there may be a sub-population of cells post-transduction which express an activity modulator in the absence of a CAR. Where each of the vectors in the mixture of viral vectors comprises a nucleic acid sequence encoding a CAR, it may not be necessary to purify or sort cells on the basis of CAR-expression, as it should not be possible for any cells to express an activity modulator in the absence of a CAR.

### PHARMACEUTICAL COMPOSITION

The cell composition of the present invention, comprising a mixture of singly and combinatorially transduced CAR-expressing cells, may be administered to a patient as a pharmaceutical composition.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

### METHOD OF TREATMENT

The present invention provides a method for treating a disease which comprises the step of administering the a cell composition of the present invention (for example in a pharmaceutical composition as described above) to a subject.

A method for treating a disease relates to the therapeutic use of the cell composition of the present invention. The cell composition may be administered to a subject having an existing disease or condition in order to lessen, reduce or improve at least one symptom associated with the disease and/or to slow down, reduce or block the progression of the disease.

The method for preventing a disease relates to the prophylactic use of the cell composition of the present invention. The cell composition may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
(i) isolating a cell-containing sample;
(ii) transducing the such cells with a mixture of at least two viral vectors;
(iii) administering the cells from (ii) to a subject.

The present invention also provides a cell composition of the present invention for use in treating and/or preventing a disease.

The invention also relates to the use of a cell composition of the present invention in the manufacture of a medicament for the treatment of a disease.

The disease to be treated by the methods of the present invention may be a cancerous disease, such as bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

The disease may be Multiple Myeloma (MM), B-cell Acute Lymphoblastic Leukaemia (B-ALL), Chronic Lymphocytic Leukaemia (CLL), Neuroblastoma, T-cell acute Lymphoblastic Leukaemia (T-ALL) or diffuse large B-cell lymphoma (DLBCL).

The disease may be a plasma cell disorder such as plasmacytoma, plasma cell leukemia, multiple myeloma, macroglobulinemia, amyloidosis, Waldenstrom's macroglobulinemia, solitary bone plasmacytoma, extramedullary plasmacytoma, osteosclerotic myeloma, heavy chain diseases, monoclonal gammopathy of undetermined significance or smoldering multiple myeloma.

The cells of the composition of the present invention may be capable of killing target cells, such as cancer cells. The target cell may be characterised by the presence of a tumour secreted ligand or chemokine ligand in the vicinity of the target cell. The target cell may be characterised by the presence of a soluble ligand together with the expression of a tumour-associated antigen (TAA) at the target cell surface.

Different supopulations of cells within the composition of the invention may have different levels of ability to kill target cells, both between different patients having the same disease and within a patient at different disease sites (e.g. tumour sites).

### Results-Based Engineering of Car-Expressing Cells

The present invention also provides a method for determining the optimal combination of components for a CAR-expressing cell to treat a disease.

As explained above, the cell composition of the invention comprises a mixture of CAR-expressing cells, some of which are single transduced and some of which are combinatorially transduced. The number of different subpopulations will depend on the number of viral vectors in the mixture used for transduction. For three vectors A, B and C, there should be seven populations of transduced cells: those transduced with: A alone; B alone; C alone; A and B; A and C; B and C; and A, B and C. For four vectors, A, B, C and D, there should be 15 different subpopulations: A alone; B alone; C alone; D alone; A and B; A and C; A and D; B and C; B and D; C and D; A, B and C; A, B and D; A, C and D; B, C and D; and A, B, C and D.

If each vector in the composition comprises a nucleic acid encoding a CAR and/or a nucleic acid encoding an activity modulator, then the cell composition will be a mixture subpopulations of transduced cells each expressing a different combination of CAR(s) and activity modulator(s).

For example, the mixture of viral vectors may express one or more CAR(s) and a plurality of different activity modulators. CAR-expressing cells made by transduction of cells with the mixture of vectors will express a plurality of different combinations of activity modulator(s) which may give the CAR-expressing cell different properties in terms of target cell killing, survival, engraftment, resistance to checkpoint inhibition and/or resistance to the hostile tumour microenvironment. In vivo, one subpopulation of cells expressing a particular combination of CAR(s) and activity modulators will be best suited to the particular conditions within the patient or within a particular site in the patient. That subpopulation will receive the most effective activation, survival and/or proliferation signal and will “win out” from other subpopulations in the cell composition.

By analysing CAR-expressing cells in a patient following administration, it is possible to work out which subpopulation is best survive, engraft and kill target cells. By analysing the phenotype or genotype of this subpopulation, it is then possible to work out which combination of vectors the subpopulation were successfully transduced with during the making of the cell composition.

This information may be used to design a homogenous CAR-expressing cell composition, in which every transduced cells expresses the most successful combination of CAR(s) and activity modulator(s).

The method may comprise the following steps:
(i) administering a cell composition to a subject having the disease;
(ii) monitoring the patient or sample(s) from the patient to determine which sub-population of cells in the cell composition show the greatest level of survival, engraftment, proliferation activation and/or target cell killing; and
(iii) analysing the phenotype/genotype of the cells in the sub-population to ascertain the CAR(s) and/or activity modulator(s) expressed by those cells.

There is also provided a method for producing a CAR-expressing cell composition for use in the treatment of a disease which comprises the step of determining the optimal combination of components for a CAR-expressing cell to treat a disease by a method described above, then transducing cells with a single vector expressing the identified combination of components. The resulting cell composition will be homogeneous in the sense that all CAR-expressing cells will express the same combination of components.

It is also possible to transducing cells with two or more vectors expressing the identified combination of components, then sort for cells which express all identified components in order to arrive at a homogeneous CAR-expressing cell composition.

The present invention also provides a cell composition made by such a method.

The invention will now be further described by way of Examples, which are meant to serve to assist one of ordinary skill in the art in carrying out the invention and are not intended in any way to limit the scope of the invention.

### EXAMPLES

### Example 1—Generation of a CAR-T Cell Composition Transduced with Multiple Vectors

Lentiviral vectors were generated expressing either a) the second-generation anti-CD19 CAR described in WO2016/139487 which comprises an anti-CD19 antigen binding domain, a CD8 stalk spacer and transmembrane domain, and a compound 4-1BB-CD3ζ endodomain under the control of a PGK promoter (pCCL.PGK.aCD19cat-CD8STK-41BBZ); or b) an anti-CD22 CAR having CDRs shown as SEQ ID No. 10-15 above, a CD8 stalk spacer and a second generation endodomain comprising CD33 and a 4-1BB costimulatory domain, under the control of an EF1α promoter (pCCL.EF1a.aCD22_9A8-1-64_LH_scFv-CD8STK-41BBz).

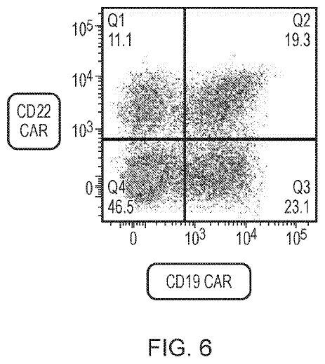

Two separate lentiviral supernatants were produced and mixed 1:1 at an MOI of 2.5+2.5. The pattern of expression of the two CARs was investigated by flow cytometry using an anti-idiotype antibody to stain for expression of the anti-CD19 CAR and soluble CD22 to stain for expression of the CD22 CAR. The results are shown in FIG. 6. Post-transduction with the lentiviral composition, the cells are a mixture of untransduced cells (46.5%); cells expressing the anti-CD19 CAR alone (23.1%); cells expressing the anti-CD22 CAR alone (11.1%) and cells expressing both the CD19 and CD22 CARs (19.3%).

### Example 2—Expression of CAR(s) in Combination with Enhancement Module(s) in a Combinatorial Fashion by Transduction with Multiple Vectors

Vectors are constructed which contain a second generation anti-CD19 CAR (Fmc63-41BBz) in combination with a marker gene and one of the following enhancement modules, which are predicted to display activity under specific conditions:
dnSHP2—A truncated version of SHP2 which has the SH2 domains but lacks the phosphatase domain. The sequence is shown above as SEQ ID No. 29. The truncated protein acts as a dominant negative and competes with the wild-type proteins for binding to phosphorylated ITIM on inhibitory immunoreceptors such as PD1.
dnTGFβRII—A dominant negative TGFβ receptor which lacks the kinase domain. The sequence is shown above as SEQ ID No. 46. The dnTGFβRII competes with wild-type TGF-β receptor for binding to TGF-β, downregulating TGFβ-mediated signalling.
CCR—A constitutively active chimeric cytokine receptor having an IL-2, IL-7 or GM-CSF receptor endodomain, as described above. The sequences for components of for such constitutively active CCRs are given above as SEQ ID No. 30-43. The present of such a receptor causes constitutive cytokine signalling (i.e. in the absence of the relevant cytokine).

Retroviral vectors are constructed containing the following cassettes, in which GFP, mKate and RQR8 are marker genes:
Vector A—CAR+dnSHP+GFP
Vector B—CAR+dnTβRII+mKate
Vector C—CAR+CCR+RQR8

T cells are transduced with a mix of two retroviruses encoding two of the above vectors in a 1:1 mixture at an MOI of 2.5:2.5. The resulting mixed population contains T cells expressing the first vector OR the second vector OR both vectors together. Transduction is detected and measured by flow cytometry measuring the expression of the marker genes. For example, for cells transduced with a mixture of vectors A and B, transduction is detected and measured using the marker genes GFP and mKate.

The transduced T cell population is labelled with the dye Cell Trace Violet (CTV), a fluorescent dye which is hydrolysed and retained within the cell. It is excited by the 405 nm (violet) laser and fluorescence can be detected in the pacific blue channel. The T-cells are resuspended at 2×10⁶ cells per ml in PBS, and 1 ul/ml of 5 mM CTV is added. The T-cells are incubated with the CTV for 20 minutes at 37° C. Subsequently, the cells are quenched by adding 5V of complete media. After a 5 minutes incubation, the T-cells are washed and resuspended in 2 ml of complete media. An additional 10 minute incubation at room temperature allows the occurrence of acetate hydrolysis and retention of the dye.

Labelled T-cells are co-cultured with CD-19 expressing target cells for four or seven days. In order to investigate the function of the dnSHP2 enhancement module in vector mixtures comprising Vector A, target cells are used which co-express the target antigen and PDL1. In order to investigate the function of the dnTβRII enhancement module in vector mixtures comprising vector B, T-cells are co-cultured with target cells in the presence of soluble TGF-β.

The assay is carried out in a 96-well plate in 0.2 ml total volume using 5×10⁴ transduced T-cells per well and an equal number of target cells (ratio 1:1). At the day four or day 7 time point, the T-cells are analysed by flow cytometry to measure the dilution of the CTV which occurs as the T-cells divide. The number of T-cells present at the end of the co-culture is calculated, and expressed as a fold of proliferation compared to the input number of T cells.

Preferential expansion in the module corresponding to the immunosuppression exerted by the target cells is assessed by a decrease in cell trace violet dye in that population and by an increase in the marker corresponding to that population.

### Example 3—Generation of an Anti-GD2-CAR T-Cell Product with Enhancement Modules by Dual Transduction with Two Separate Retroviral Vectors

Background: Neuroblastoma is the most common extracranial solid cancer in children with poor long-term survival in those with high-risk disease.

A currently ongoing phase I clinical study of GD2-targeted CART for refractory/relapsed neuroblastoma (NCT02761915) shows activity against disseminated disease without inducing on target/off tumor toxicity. However, CART persistence was limited and clinical activity transient and incomplete.

Building on the GD2 CAR used in this study, we have developed a next generation T-cell product candidate termed AUTO6NG. The AUTO6NG product consists of 3 distinct populations of GD2-targeted CAR T-cells, produced by dual transduction of T-cells with two separate retroviral vectors. The first vector directs the expression of a GD2-targeting CAR, co-expressed with a constitutively signalling IL7 cytokine receptor (IL7R_CCR) or a constitutively signalling IL2 cytokine receptor (IL2R_CCR) (product A), while the second vector is a tri-cistronic retroviral vector encoding the same GD2 CAR, co-expressed with dominant negative TGFbRII (dnTGFbRII) and truncated SHP2 (dSHP2) (product B). dSHP2 confers resistance to inhibitory signals such as those from PD1.

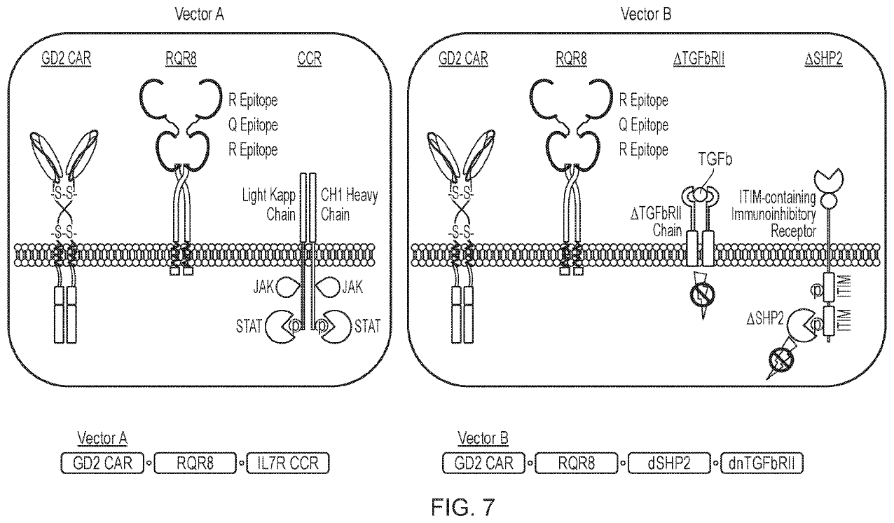

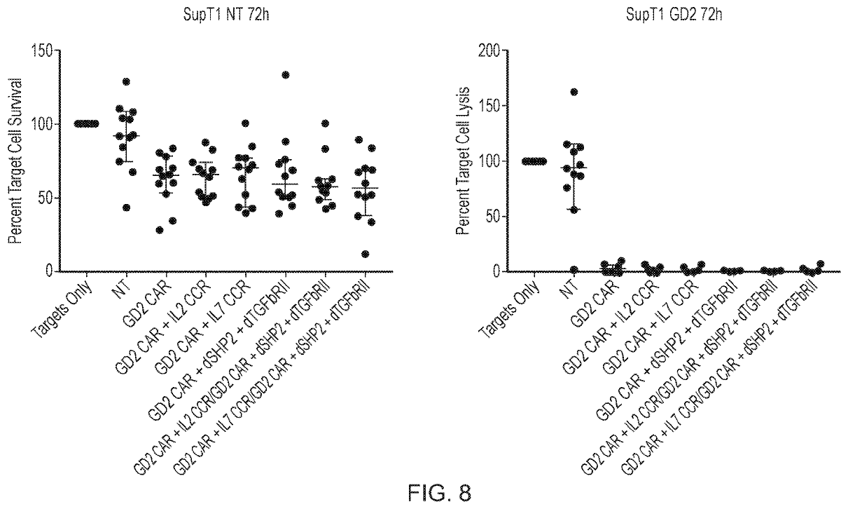

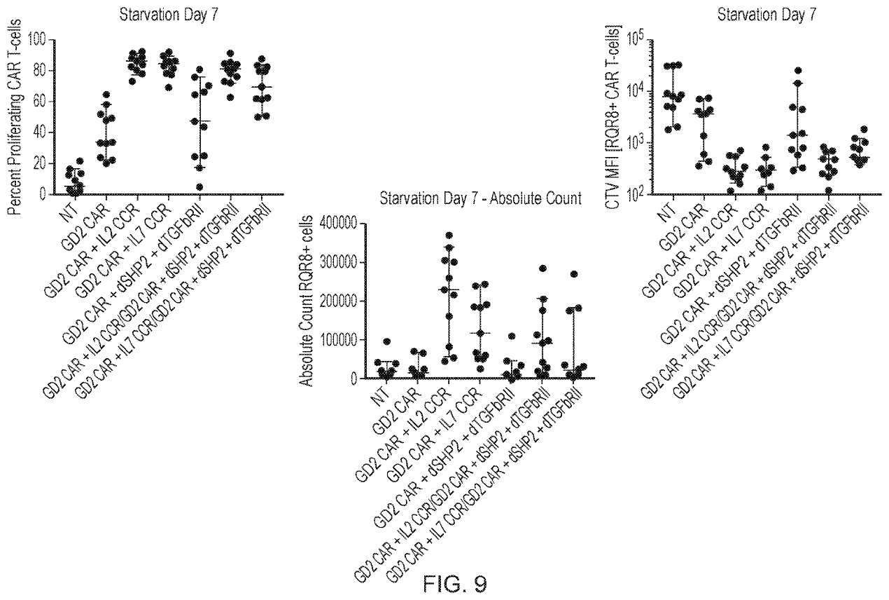

The vector design is illustrated schematically in FIG. 7.

The GD2 CAR is as described in WO2015/132604, with an antigen-binding domain with a VH domain having the sequence shown as SEQ ID No. 77 and a VL domain having the sequence shown as SEQ ID No. 78.

The constitutively signalling IL2 and IL7 cytokine receptors are as described in WO2017/029512, The IL2 CCR comprises a comprising a first polypeptide having an IL-2 receptor β-chain endodomain (SEQ ID No. 40) and a second polypeptide comprising a common γ-chain receptor endodomain (SEQ ID No. 39); whereas the IL7 CCR comprises a first polypeptide having an IL-7 receptor α-chain endodomain (SEQ ID No. 41) and a second polypeptide comprising a common γ-chain receptor endodomain (SEQ ID No. 39).

The sort/suicide gene RQR8 is as described in WO2013/153391 and has the sequence shown as SEQ ID No. 79.

The dominant negative TGFbRII (dnTGFbRII) has the sequence shown above as SEQ ID No. 46.

The truncated SHP2 (dSHP2) has the sequence shown above as SEQ ID No. 29.

### Example 4—Investigating the Cytotoxic Capacity of Single and Dual Transduced Cells And the Function of Various Vector-Expressed Elements In Vitro

Human T-cells were either dual transduced with both vectors yielding a mix of product A/B/A+B (AUTO6NG) or single transduced with each vector individually giving raise to product A or B. Controls included non-transduced cells (NT) and cells expressing GD2 CAR alone.

### i) Cytotoxicity Assay

The various effector cell types were co-cultured with GD2-expressing SupT1 target cells (SupT1 GD2) or control, non-transduced target cells (SupT1 NT) for 72 hours and the percentage of target cell lysis was analysed by flow cytometry. The results are shown in FIG. 8. All CAR-expressing effector cells were capable of killing GD2-expressing target cells. T-cells transduced with the dual vector composition (product A/B/A+B) were highly potent in cytotoxicity assays against GD2 positive tumour cell lines with no differences observed compared with single transduced CAR T-cells (product A or B).

### ii) Validation of the CCR

The various transduced CAR T-cells described above and control NT T-cells were labelled with Cell Trace Violet (CTV) and cultured in cytokine-free complete cell culture media for 7 days without further antigen stimulus. In vitro persistence was quantified as percent proliferating cells which have diluted the CTV dye and absolute CAR T-cell count after 7 days. The results are shown in FIG. 9. T cells transduced with product A, expressing either the constitutively signalling IL7 cytokine receptor (IL7R_CCR) or the constitutively signalling IL2 cytokine receptor (IL2R_CCR); or transduced with product A+B showed increased proliferation compared with untransduced cells (NT), cells transduced with a vector expressing GD2 CAR alone, or cells transduced with the vector expressing product B alone (GD2 CAR+dSHP2+dTGFbRII). Expression of either the IL2 or IL7R_CCR conferred exogenous-cytokine-independent viability and homeostatic proliferation of modified T-cells, without causing autonomous T-cell growth.

iii) Validation of the dTGFbRII

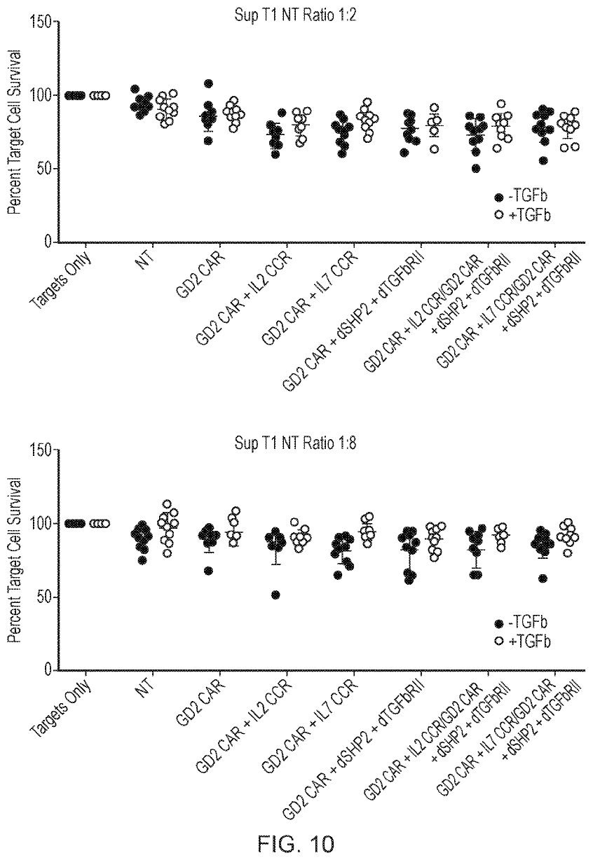

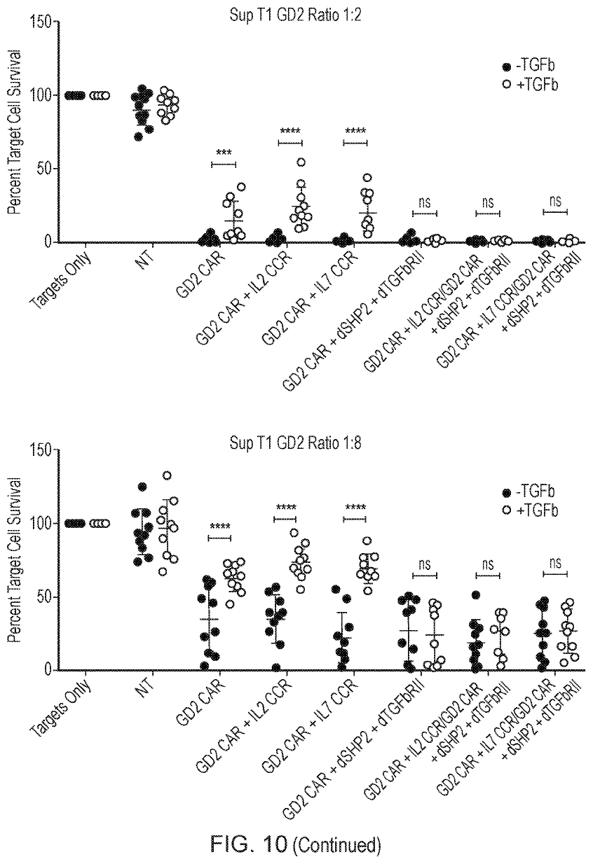

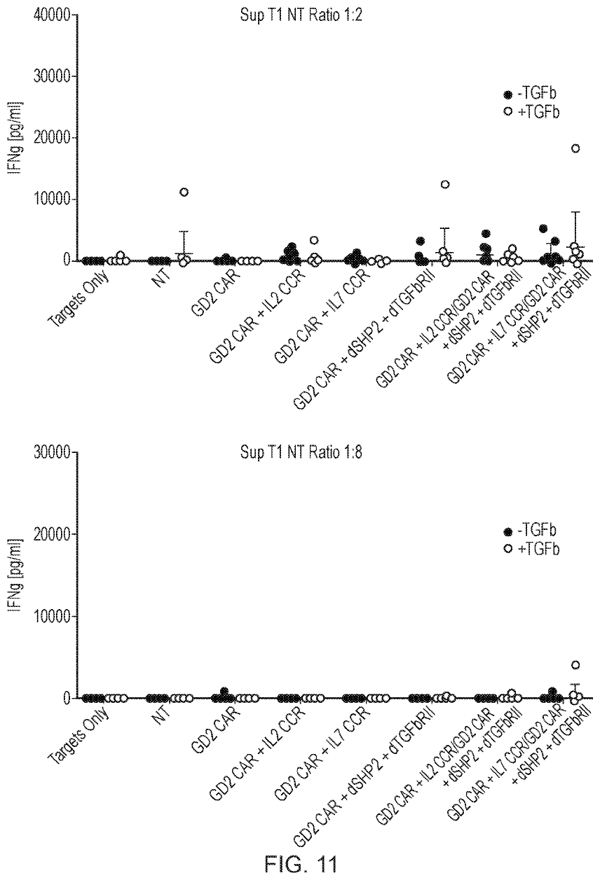

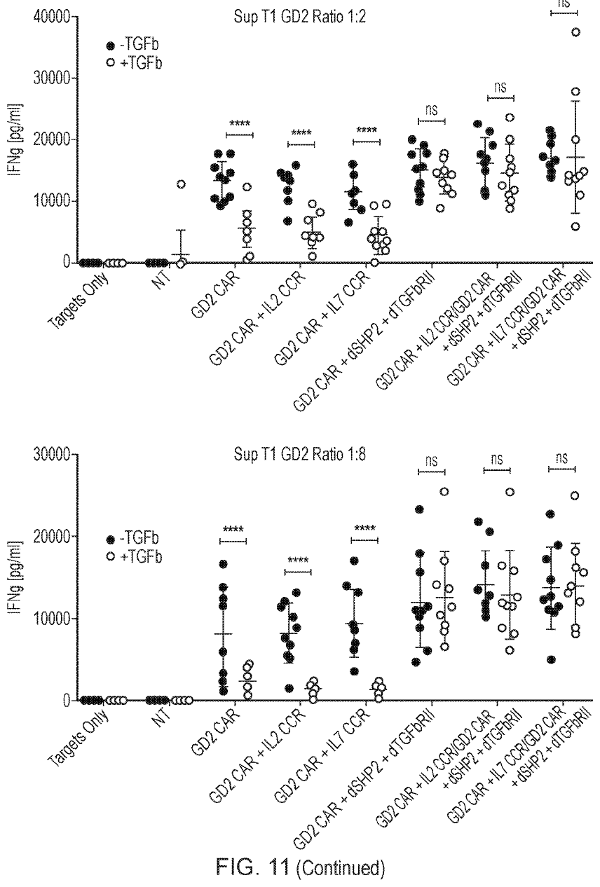

The various transduced CAR T-cells described above and control NT T-cells were co-cultured with GD2-expressing SupT1 target cells (SupT1 GD2) or control, non-transduced target cells (SupT1 NT) at a 1:2 or 1:8 E:T ratio, for 7 days, in the presence or absence of 10 ng/ml TGFβ. Killing of target cells was analysed by flow cytometry and secretion of IFNγ was analysed by ELISA. The results are shown in FIGS. 10 and 11 respectively. CAR T cells transduced with product B, expressing dnTGFbRII; or transduced with product A+B, showed resistance to TGFβ-mediated inhibition of target cell killing compared with CAR-T cells expressing GD2 CAR alone, or cells transduced with the vector expressing either product A alone (GD2 CAR+IL2 CCR or GD2 CAR+IL7 CCR). CAR T cells transduced with product B or product A+B, showed restoration of IFNγ secretion in the presence of TGFβ to a level comparable to that observed in the absence of TGFβ. By contrast, IFNγ secretion by CAR-T cells expressing GD2 CAR alone, or cells transduced with the vector expressing either product A alone, was significantly inhibited by the presence of TGFβ. T-cells expressing dnTGFbRII therefore proved resistant to TGFβ-mediated immunosuppression in vitro.

### Example 5-Investigating Anti-Tumour Activity of Dual-Transduced CAR-T Cell Product In Vivo in an Xenograft Model of Neuroblastoma

An in vivo assay was used to investigate the anti-tumour activity of T cells transduced with a dual vector composition by intravenous administration in an established neuroblastoma xenograft model in NSG mice. Ten- to 14-week-old female NSG mice were intravenously injected with 1 million Firefly luciferase expressing CHLA-255 cells (CHLA-255 FFluc). Xenografts were left to establish for 15 days until stable engraftment was detectable by BLI. CAR-T cells were made either by transducing cells with a single vector expressing a GD2 CAR (GD2 CAR) or by transducing cells with the dual vector composition described in Example 3 and Illustrated in FIG. 7 (GD2 CAR+IL7 CCR/GD2 CAR+dSHP2+dTGFbRII). Mice were injected intravenously with 10×10⁶ CAR T-cells (50% transduction efficiency), 3×10⁶ CAR T-cells (50% transduction efficiency), 20×10⁶ NT T-cells (total T-cells equivalent to 10×10⁶ CAR T-cell dose) or PBS. Fourteen days later, tumour growth was assessed by bi-weekly bioluminescent imaging.

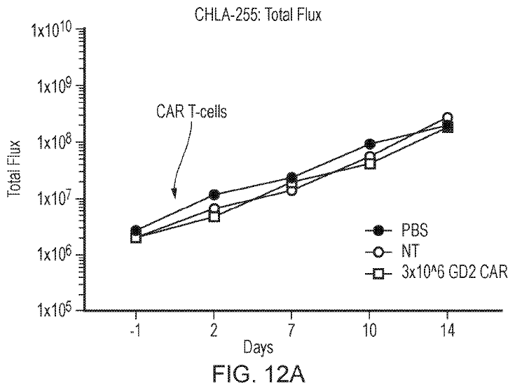

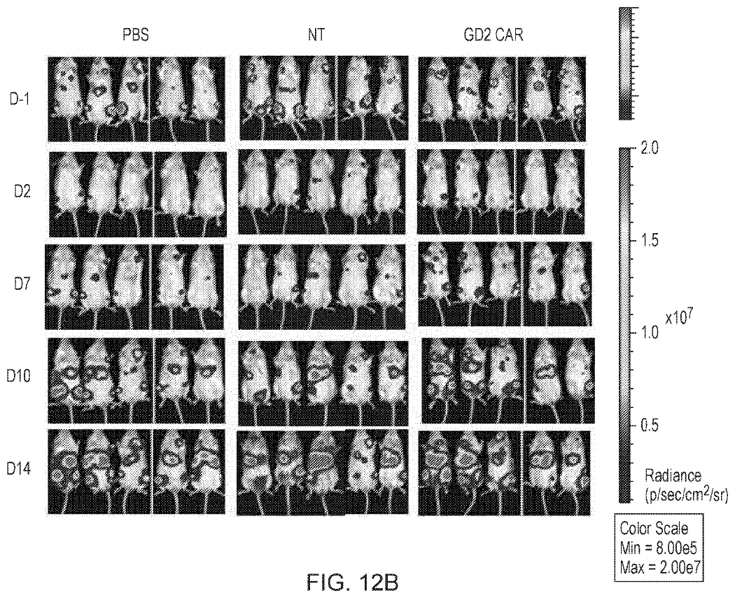

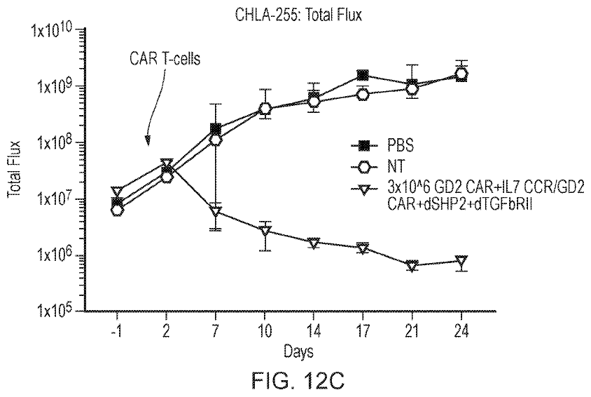

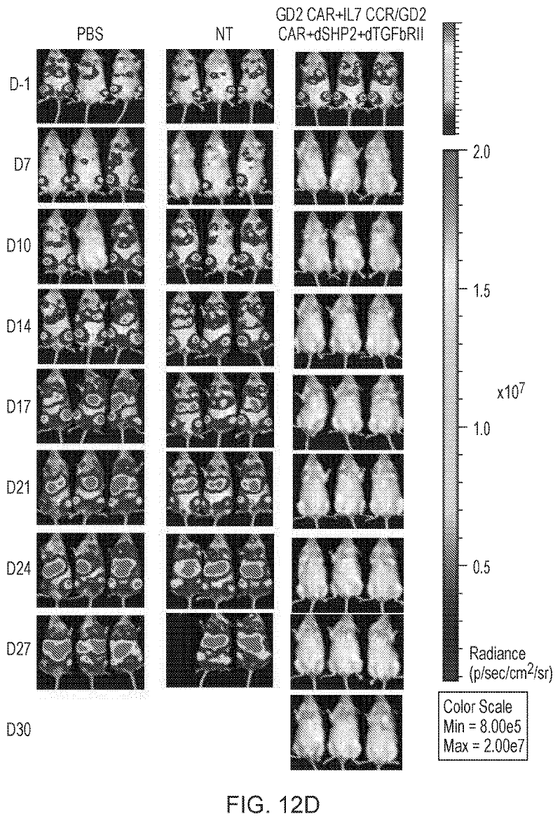

The results are shown in FIG. 12. Intravenous delivery of CAR T cells expressing a simple GD2 CAR alone had no significant effect on tumour growth (FIGS. 12A and B). By contrast, intravenous delivery of CAR T cells transduced with the dual vector composition at both the 3×10⁶ and 10×10⁶ doses exhibited potent anti-tumour activity and extended survival in NSG mice with established tumour burden (FIGS. 12C and D).

### Example 6—Generation of an Anti-PSMA CAR T-Cell Product with Enhancement Modules by Triple Transduction with Three Separate Retroviral Vectors

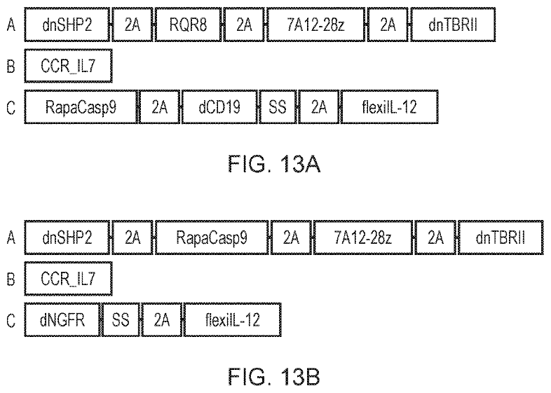

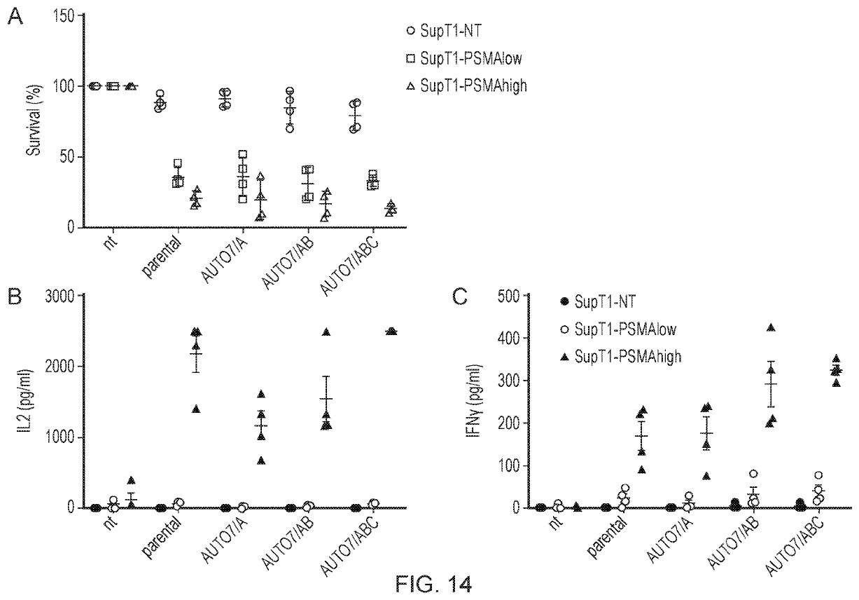

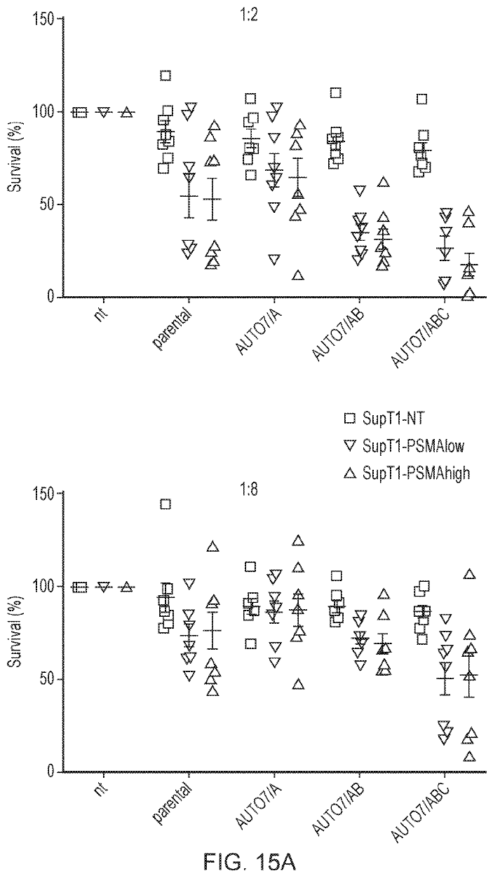

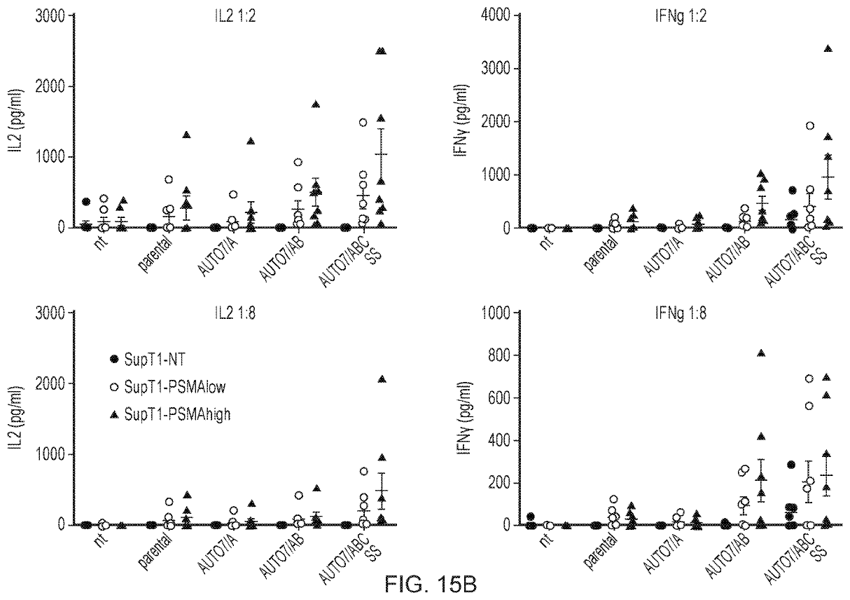

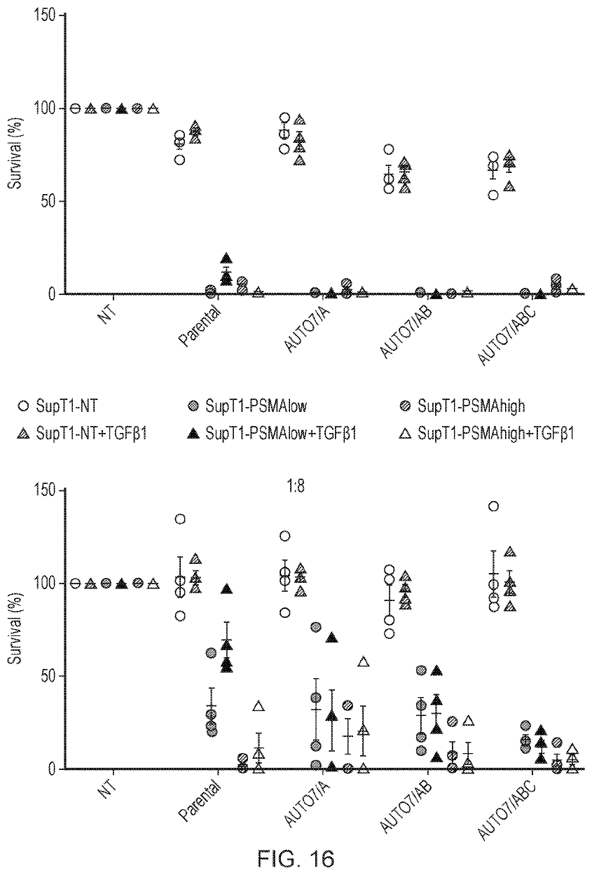

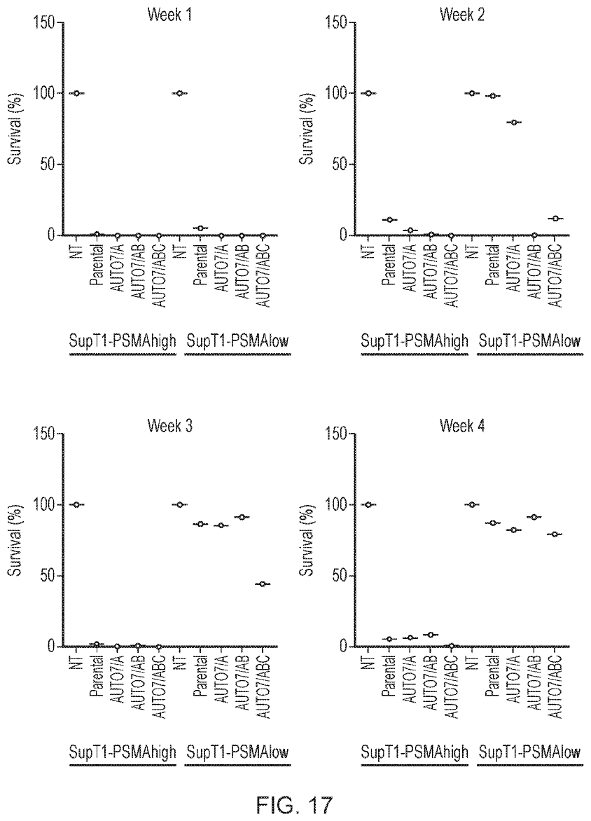

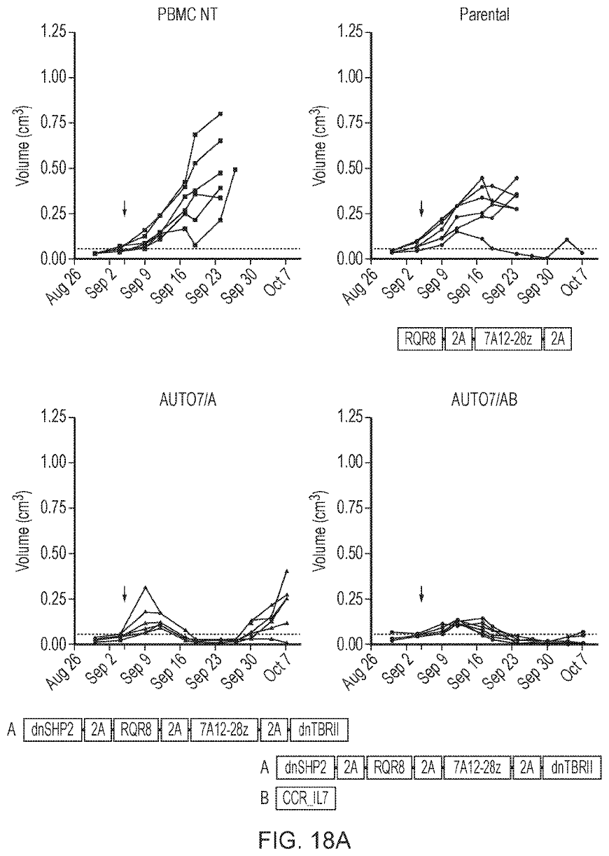

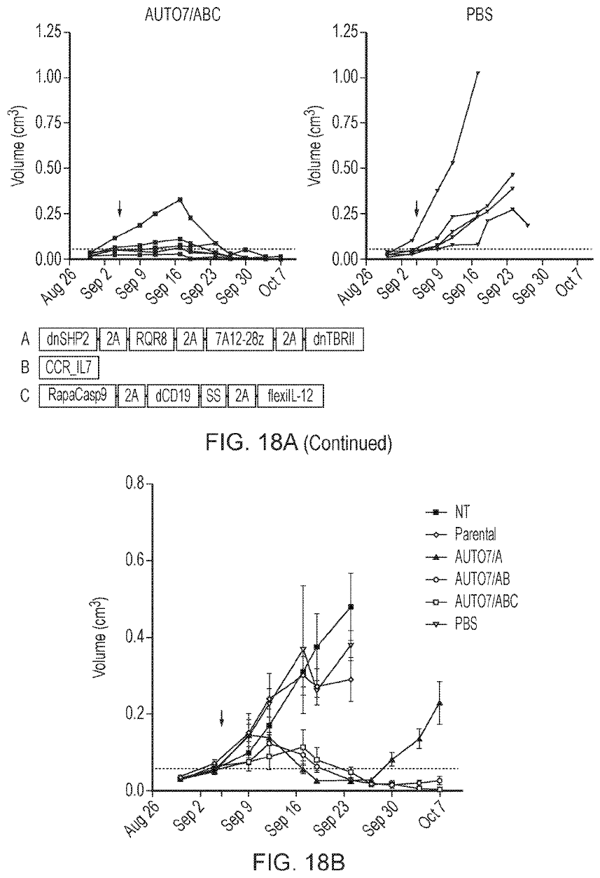

The present inventors developed a combinatorial/multi-modular CAR T therapeutic for the treatment of prostate cancer (“AUTO7”) which consists of several functional modules: 1) an anti-PSMA CAR having a second generation CD28-CD3z compound endodomain, (7A12-28z); 2) a safety switch, RapaCasp9 which is described in WO2016/135470 and has the sequence shown as SEQ ID No. 80 above; 3) dominant negative TGFβRII to induce TGFβ1 resistance (dnTβRII) having the sequence shown as SEQ ID No. 46 above; 4) truncated SHP2 (dnSHP2) for PD1/PD-L1 pathway inhibition having the sequence shown as SEQ ID No. 29 above; 5) a constitutively active IL7 receptor (CCR_IL7) to induce proliferation as described above; 6) a sort-suicide gene RQR8 having the sequence shown as SEQ ID No. 79; and 7) an ultra-low-secreting IL-12 (flexilL-12) for lymphocyte recruitment/activation which is positioned downstream of a stop-skip sequence (SS) which has the sequence shown as SEQ ID No. 81. The vector design is shown in FIG. 13A.

The first vector (A) encodes dnSHP2, the suicide gene RQR8, the CAR and dnTβRII; the second vector (B) encodes the constitutively active IL7 receptor; the third vector (C) encodes a second suicide gene: Rapcasp9; and flexi-IL12.

Having different suicide genes on vector A and vector C provides flexibility. If a CAR-related toxicity is observed in the patient, then it is possible to selectively remove cells expressing the CAR in the usual way treating the patient with Rituximab, which triggers apoptosis of these cells via the RQR8 sort/suicide gene. If, however, a toxicity is observed which is thought to be related to IL-12 secretion, then cells can be selectively destroyed which are transduced with vector C by adding rapamycin or a rapamycin analogue to trigger apoptosis via the RapaCasp9 suicide gene. As the therapeutic product comprises cells transduced with a mixture of three vectors, it will be a combinatorial product, with distinct subpopulations of cells transduced with all the various combinations of one, two or all three vectors. This means that some CAR-expressing cells should survive triggering of the RapaCasp9 suicide gene. For example, cell transduced with vector A alone or the combination of vectors A and B will not express RapaCasp9 and should be unaffected by treatment with rapamycin. This means that it is possible to “modify” the therapeutic product in vivo and selectively delete the IL-12 expressing cells, while leaving a proportion of CAR-expressing cells to maintain the CAR-mediated anti-tumour effect.

AUTO7 was investigated as product of a single transduction using the vector A. (“AUTO7/A”), or double transduction using vectors A and B (“AUTO7/AB”), or triple transduction using vectors A, B and C (“AUTO7/ABC”). AUTO7 was tested against a second generation CAR developed using the same anti-PSMA binder 7A12 (“Parental”).

### Example 7—Investigating the Cytotoxic Capacity of Single, Dual and Triple Transduced Cells and the Function of Various Vector-Expressed Elements In Vitro

The capacity of the T-cells transduced with the single, dual and triple vector combinations described above to kill target cells was investigated using a FACS-based killing assay. SupT1 cells engineered to express human PSMA antigen at different levels (SupT1-PSMAhigh, SupT1-PSMAlow) were used as target cells. Non-engineered SupT1 cells (SupT1-NT) were used as a negative control. CAR T-cells were co-cultured with target cells at 1:2 effector to target ratios. FBK was assayed after 24 h of incubation by cytofluorimetry analysis and the results are shown in FIG. 14A. Secretion of IL-2 and IFNγ by CAR T-cells was measured by collecting supernatant at 24 hr from the co-cultures described and detected by ELISA and the results are shown in FIGS. 14B and C respectively.

It was found that cell compositions produced by single transduction using the vector A. (“AUTO7/A”), double transduction using vectors A and B (“AUTO7/AB”), or triple transduction using vectors A, B and C (“AUTO7/ABC”) were all highly potent in cytotoxicity assays against PSMA positive tumour cell lines. Cytotoxicity and cytokine release were comparable to those observed with a second generation CAR developed using the same anti PSMA binder 7A12 (“Parental”) which was used as a CAR alone control.

In order to investigate the capacity of the single, dual and triple transduced AUTO7 T cells to kill target cells following culture in the absence of IL-2, transduced T-cells were cultured in starvation conditions with media without the supplement of IL2 for 7 days. After 7 days CAR T-cells were counted and plated together with SupT1-PSMAhigh and SupT1-PSMAlow targets cells (or SupT1-NT cells as negative control). CAR T-cells were co-cultured with target cells at 1:2 and 1:8 effector to target ratios. FBK was assayed after 24 h of incubation by cytofluorimetry analysis (FIG. 15A). Secretion of IL-2 and IFNγ by CAR T-cells was measured by collecting supernatant at 24 hr from the co-cultures described and detected by ELISA (FIG. 15B).

It was found that, although cells expressing the control CAR and cells transduced with vector A alone showed in inhibition in cytotoxicity and cytokine release following culture under starvation conditions, this effect was less pronounced with cells transduced with the dual or triple vector combination (vectors A+B or A, B+C). Vector B comprises a gene expressing a constitutively active cytokine receptor: the IL7R_CCR module. Expression of this module confers cytokine independent viability and proliferation, without interfering with the cytotoxicity; since after starvation, cells transduced with the AUTO7/AB or AUTO7/ABC were shown to be still efficient at killing PSMA expressing target cells.

In order to investigate the capacity of the single, dual and triple transduced AUTO7 T cells to kill target cells in the presence of TGFβ, transduced T-cells were co-cultured with SupT1-PSMAhigh and SupT1-PSMAlow targets for 7 days at ratio 1:2 and 1:8 (E:T) either in the presence or absence of 10 ng/ml TGFβ1 (SupT1-NT were used as control). Target cell killing was quantified by FACS and normalised to targets alone. The results are shown in FIG. 16.

At a 1:8 E:T ratio, killing of target cells by T cells expressing the CAR alone control was inhibited by the addition of TGFβ1 to the culture medium. This inhibitory effect was reduced for cells transduced with any of the vector combinations A; A+B; or A, B+C. Vector A encodes the dnTGFβRII element which has been previously shown to block TGFβ-mediated inhibition of T-cell signalling.

In order to investigate the capacity of the single, dual and triple transduced AUTO7 T cells to kill target cells following repeated exposure to target antigen, transduced cells were co-cultured with SupT1-PSMAhigh or SupT1-PSMAlow target cells at 1:1 ratio (E:T) and every 7 days CAR T-cells were re-stimulated with 5×10⁴ SupT1 cells. Target cell killing was quantified by FACS before each new re-stimulation. The results are shown in FIG. 17.

After two restimulation events (week 2) cells transduced with the vector combination A+B or A+B+C showed much better killing of target cells than CAR-alone or Vector A-transduced cells. After three restimulation events (week 3) cells transduced with the vector combination A+B+C showed the best killing of target cells.

### Example 8—Investigating the Cytotoxic Capacity of Single, Dual and Triple Transduced Cells and the Function of Various Vector-Expressed Elements In Vivo

An in vivo assay was used to investigate the anti-tumour activity of T cells transduced with a single, dual or triple vector composition by intravenous administration in an established xenograft model in NSG mice.

Female NSG mice were injected with 5×10⁶ PSMA positive PC3 human cell lines in the flank. Xenografts were left to establish for 3 weeks until stable engraftment was detectable by palpation and calliper measurement. CAR T-cells were administered i.v. at a dose of 1×10⁶ CAR T-cells/mouse. Calliper measurement was taken 2/3 times a week. The results are shown in FIG. 18.

Mice treated with T-cells expressing the CAR alone (Parenteral) showed slowed tumour growth than those receiving non-transduced cells, but the tumour growth was not controlled. Mice receiving T cells transduced with vector A, which encodes CAR, dnSHP2 and dnTGFβRII, showed an initial growth in tumour (September 9) followed by a reduction in tumour size (Sep23). At the end of the study, some resumption of tumour growth was observed (October 7).

Mice receiving T cells transduced with vector A+B showed an initial growth in tumour (September 9-16), but then the tumour size reduced and this effect continued for the duration of the study. Triple transduced AUTO7 CAR T cells completely eradicated tumour with no signs of toxicity. The vector combinations, especially A+B and A+B+C show a significant improvement over CAR alone in reducing tumour growth and promoting survival of mice in a xenograft model.

The results demonstrate the feasibility and efficacy of the multi-modular AUTO7 product. Addition of IL7R_CCR, ss_fIL12, dnTGFβRII and dSHP2 modules to the anti-PSMA CAR product enhanced T cell functions by extending persistence, proliferation, activation and resistance to TGFβ1 and PD1/PDL1 driven immune inhibition.

### Methodology

### Binder Generation

PSMA-binder was generated by CDR grafting of an anti-PSMA antibody derived from genetically vaccinated rats.

### Cell Lines

PC3 cells, SupT1 cell lines (NT and PSMA+) were cultured in RPMI-1640 Medium supplemented with 10% fetal bovine serum (FBS) and 1% GlutaMAX. T-cells were isolated from peripheral blood mononuclear cells (PBMCs) and maintained in RPMI-1640 Medium supplemented with 10% FBS, 1% GlutaMAX and 100 U/mL IL-2.

### Transduction

Retrovirus was generated by transiently transfecting HEK293T cells using GeneJuice with RDF plasmid (RD114 envelope), gag/pol plasmid and CAR plasmid. Retroviral viral supernatant was harvested at 48 and 72 hours. T cells were stimulated using 0.5 μg/mL of anti-CD3 and anti-CD28 antibodies in T175 TC-treated flasks and maintained in 100 U/mL IL-2. Non-TC treated six-well plates were coated with Retronectin and incubated at 4° C. for 24 hours prior to T cell transduction. A total of 3 ml of viral supernatant/supernatants was plated prior to the addition of 1 ml of activated T cells at a concentration of 1×10⁶ cells/ml, 100 U/ml of IL-2 was then added and centrifuged at 1000×g for 40 minutes at room temperature and incubated at 37° C. and 5% CO₂ for 2-3 days.

Human T-Cells were:
triple transduced with vector A., B. and C. yielding a mix of product (AUTO7/ABC)·
dual transduced with vector A. and B. yielding a mix of product (AUTO7/AB)·
single transduced with vector A. yielding a single product (AUTO7/A)

### Cytotoxicity Assay

CAR T-cells were co-cultured with SupT1-NT and SupT1-PSMA at an effector to target ratio (E:T ratio) of 1:2 or 1:8 a TC-treated 96-well plate. Readouts were taken 24 hours post co-culture by staining with anti-CD3-PeCy7 and Qben10-APC to differentiate effector T-cells and target cells, SYTOX Blue7-AAD dead cell stain was used to exclude dead cells. Cytotoxicity readouts were accessed by flow cytometry.

### Cytotoxicity Assay in Presence of TGF

CAR T-cells were co-cultured with SupT1-NT and SupT1-PSMAhigh and SupT1-PSMAlow at an effector to target ratio (E:T ratio) of 1:2 or 1:8 a TC-treated 96-well plate. TGFβ1 was added at a concentration of 10 ng/ml on day 0, and cytotoxicity readouts were accessed by flow cytometry on day 7.

### In Vitro Re-Stimulation Assay

CAR T-cells were co-cultured with SupT1-PSMAhigh or SupT1-PSMAlow target cells at 1:1 ratio (E:T). Every 7 days CAR T-cells were re-stimulated with 5×10⁴ SupT1 cells. Cytotoxicity was assessed by FBK assay as describe above, and target:effectors ratio after re-stimulation was ascended by cytofluorimetric analysis. Supernatants were collected to assess cytokine release.

### Cytokine ELISA

Human IL-2 ELISA MAX™ Deluxe and Human IFN-γ ELISA MAX™ Deluxe kits were used to access the levels of cytokine secreted into co-culture supernatants taken from Cytotoxicity Assay.

### In Vivo Experiment

5×10⁶ PSMA positive PC3 human cell lines were injected in the flank of female NSG mice. Xenografts were left to establish for 3 weeks until stable engraftment was detectable by palpation and calliper measurement. Human PBMC were created by single, double or triple transduction. CAR T-cells were administered i.v. at a dose of 1×10⁶ CAR T-cells/mouse. Calliper measurement was taken 2/3 times a week and followed up for until the end of the animal protocol.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1-21. (canceled)

22. A cell composition made by transducing a population of cells with a mixture of at least two viral vectors, wherein at least one vector comprises a nucleic acid sequence which encodes a chimeric antigen receptor (CAR); and wherein at least one vector comprises a nucleic acid encoding an activity modulator which modulates the activity of the CAR, of a cell expressing the CAR, or of a target cell

23. The cell composition according to claim 22, wherein the activity modulator is a dominant negative SHP-1 or SHP-2.

24. The cell composition according to claim 22, wherein the activity modulator is a dominant negative transforming growth factor (TGF)β receptor.

25. The cell composition according to claim 22, wherein the activity modulator is a constitutively active chimeric cytokine receptor.

26. The cell composition according to claim 22, wherein in the mixture of viral vectors at least one vector comprises a nucleic acid sequence which encodes a dominant negative SHP-1 or SHP-2; and at least one vector comprises a nucleic acid sequence which encodes a dominant negative transforming growth factor (TGF)β receptor.

27. The cell composition according to claim 22, wherein the mixture of viral vectors comprises two, three, four, five or six viral vectors.

28. The cell composition according to claim 22, wherein the cell composition is made by, after transducing the population of cells, then selecting CAR-expressing cells from the transduced cell population.

29. The cell composition according to claim 22, wherein each of the viral vectors comprises a nucleic acid sequence encoding a CAR.

30. The cell composition of claim 29, wherein nucleic acid sequences of the viral vectors encode the same CAR.

31. The cell composition of claim 29, wherein each of the viral vectors comprises a nucleic acid encoding an activity modulator which modulates the activity of the CAR, of a cell expressing the CAR, or of a target cell.

32. The cell composition of claim 31, wherein the activity modulators are selected from a dominant negative SHP-1 or SHP-2; a dominant negative transforming growth factor (TGF)β receptor; and a constitutively active chimeric cytokine receptor.

33. The cell composition of claim 32, wherein one vector comprises a nucleic acid sequence encoding a dominant negative SHP-1 or SHP-2 and a nucleic acid sequence encoding a dominant negative transforming growth factor (TGF)β receptor; and the other vector comprises a nucleic acid sequence encoding a constitutively active chimeric cytokine receptor.

34. The cell composition of claim 30, wherein the CAR has an antigen-binding domain which binds disialoganglioside (GD2).

35. The cell composition of claim 29, wherein at least one vector comprises a nucleic acid sequence encoding a suicide gene

36. A method for treating a disease in a subject which comprises the step of administering a cell composition according to claim 22 to the subject.

37. A method for determining the optimal combination of components for a CAR-expressing cell to treat a disease, which comprises the following steps:
(i) administering a cell composition according to claim 22 to a subject having the disease,
(ii) monitoring the patient or samples from the patient to determine which sub-population of cells in the cell composition show the greatest level of engraftment and/or proliferation, and
(iii) analysing the phenotype/genotype of the cells in the sub-population to ascertain the CAR(s) and/or activity modulator(s) expressed by those cells.


SEQUENCE LISTING (81 sequences)

SEQ ID NO: 1 - protein, 7 aa, synthetic construct
  REGION 1..7: note = heavy chain variable region (VH) complementarity determining region (CDR) CDR1
GYAFSSS

SEQ ID NO: 2 - protein, 6 aa, synthetic construct
  REGION 1..6: note = VH CDR, CDR2
YPGDED

SEQ ID NO: 3 - protein, 10 aa, synthetic construct
  REGION 1..10: note = VH CDR, CDR3
SLLYGDYLDY

SEQ ID NO: 4 - protein, 10 aa, synthetic construct
  REGION 1..10: note = light chain variable region (VL) CDR, CDR1
SASSSVSYMH

SEQ ID NO: 5 - protein, 7 aa, synthetic construct
  REGION 1..7: note = VL CDR, CDR2
DTSKLAS

SEQ ID NO: 6 - protein, 9 aa, synthetic construct
  REGION 1..9: note = VL CDR, CDR3
QQWNINPLT

SEQ ID NO: 7 - protein, 119 aa, synthetic construct
  REGION 1..119: note = VH domain sequence
QVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSS

SEQ ID NO: 8 - protein, 107 aa, synthetic construct
  REGION 1..107: note = VL domain sequence
QIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKR

SEQ ID NO: 9 - protein, 20 aa, Picornavirus
RAEGRGSLLTCGDVEENPGP

SEQ ID NO: 10 - protein, 5 aa, synthetic construct
  REGION 1..5: note = VH CDR, CDR1
NFAMA

SEQ ID NO: 11 - protein, 17 aa, synthetic construct
  REGION 1..17: note = VH CDR, CDR2
SISTGGGNTYYRDSVKG

SEQ ID NO: 12 - protein, 18 aa, synthetic construct
  REGION 1..18: note = VH CDR, CDR3
QRNYYDGSYDYEGYTMDA

SEQ ID NO: 13 - protein, 11 aa, synthetic construct
  REGION 1..11: note = VL CDR, CDR1
RSSQDIGNYLT

SEQ ID NO: 14 - protein, 7 aa, synthetic construct
  REGION 1..7: note = VL CDR, CDR2
GAIKLED

SEQ ID NO: 15 - protein, 7 aa, synthetic construct
  REGION 1..7: note = VL CDR, CDR3
LQSIQYP

SEQ ID NO: 16 - protein, 127 aa, synthetic construct
  REGION 1..127: note = VH domain sequence
EVQLVESGGGLVQPGRSLKLSCAASGFTFSNFAMAWVRQPPTKGLEWVASISTGGGNTYYRDSVKGRFTISRDDAKNTQYLQMDSLRSEDTATYYCARQRNYYDGSYDYEGYTMDAWGQGTSVTVSS

SEQ ID NO: 17 - protein, 107 aa, synthetic construct
  REGION 1..107: note = VL domain sequence
DIQMTQSPSSLSASLGDRVTITCRSSQDIGNYLTWFQQKVGRSPRRMIYGAIKLEDGVPSRFSGSRSGSDYSLTISSLESEDVADYQCLQSIQYPFTFGSGTKLEIK

SEQ ID NO: 18 - protein, 254 aa, synthetic construct
  REGION 1..254: note = tyrosine kinase domain of Fyn
LQLIKRLGNGQFGEVWMGTWNGNTKVAIKTLKPGTMSPESFLEEAQIMKKLKHDKLVQLYAVVSEEPIYIVTEYMNKGSLLDFLKDGEGRALKLPNLVDMAAQVAAGMAYIERMNYIHRDLRSANILVGNGLICKIADFGLARLIEDNEYTARQGAKFPIKWTAPERALYGRFTIKSDVWSFGILLTELVTKGRVPYPGMNNREVLEQVERGYRMPCPQDCPISLHELMIHCWKKDPEERPTFEYLQSFLEDYF

SEQ ID NO: 19 - protein, 254 aa, synthetic construct
  REGION 1..254: note = tyrosine kinase domain of Src
LRLEVKLGQGCFGEVWMGTWNGTTRVAIKTLKPGTMSPEAFLQEAQVMKKLRHEKLVQLYAVVSEEPIYIVTEYMSKGSLLDFLKGETGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKEPEERPTFEYLQAFLEDYF

SEQ ID NO: 20 - protein, 254 aa, synthetic construct
  REGION 1..254: note = tyrosine kinase domain of Lck
LKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFF

SEQ ID NO: 21 - protein, 254 aa, synthetic construct
  REGION 1..254: note = tyrosine kinase domain of Lck_Y505F
LKLVERLGAGQFGEVWMGYYNGHTKVAVRSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFF

SEQ ID NO: 22 - protein, 40 aa, synthetic construct
  REGION 1..40: note = cytoplasmic tail of CD4
CVRCRHRRRQAERMSQIKRLLSEKKTCQCPHRFQKTCSPI

SEQ ID NO: 23 - protein, 32 aa, synthetic construct
  REGION 1..32: note = cytoplasmic tail of CD8
LYCNHRNRRRVCKCPRPVVKSGDKPSLSARYV

SEQ ID NO: 24 - protein, 214 aa, synthetic construct
  REGION 1..214: note = SHP-1 SH2 complete domain
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYY

SEQ ID NO: 25 - protein, 97 aa, synthetic construct
  REGION 1..97: note = SHP-1 SH2 1
WFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPL

SEQ ID NO: 26 - protein, 104 aa, synthetic construct
  REGION 1..104: note = SHP-1 SH2 2
WYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPY

SEQ ID NO: 27 - protein, 97 aa, synthetic construct
  REGION 1..97: note = SHP-2 first SH2 domain
WFHPNITGVEAENLLLTRGVDGSFLARPSKSNPGDFTLSVRRNGAVTHIKIQNTGDYYDLYGGEKFATLAELVQYYMEHHGQLKEKNGDVIELKYPL

SEQ ID NO: 28 - protein, 105 aa, synthetic construct
  REGION 1..105: note = SHP-2 second SH2 domain
WFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPL

SEQ ID NO: 29 - protein, 211 aa, synthetic construct
  REGION 1..211: note = SHP-2 both SH2 domains
WFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPL

SEQ ID NO: 30 - protein, 107 aa, synthetic construct [sequence omitted]

SEQ ID NO: 31 - protein, 19 aa, synthetic construct
  REGION 1..19: note = Human Hinge
EPKSCDKTHTCPPCPKDPK

SEQ ID NO: 32 - protein, 97 aa, synthetic construct
  REGION 1..97: note = Human CH1
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 33 - protein, 21 aa, synthetic construct
  REGION 1..21: note = Transmembrane domain from human IL2R common gamma chain
VVISVGSMGLIISLLCVYFWL

SEQ ID NO: 34 - protein, 25 aa, synthetic construct
  REGION 1..25: note = Transmembrane domain from human IL-2R beta
IPWLGHLLVGLSGAFGFIILVYLLI

SEQ ID NO: 35 - skipped

SEQ ID NO: 36 - protein, 25 aa, synthetic construct
  REGION 1..25: note = Transmembrane domain from human IL-7R alpha
PILLTISILSFFSVALLVILACVLW

SEQ ID NO: 37 - protein, 26 aa, synthetic construct
  REGION 1..26: note = Transmembrane domain from Human GF-CSFR alpha
NLGSVYIYVLLIVGTLVCGIVLGFLF

SEQ ID NO: 38 - protein, 17 aa, synthetic construct
  REGION 1..17: note = Transmembrane domain from Human GM-CSFR common beta chain
VLALIVIFLTIAVLLAL

SEQ ID NO: 39 - protein, 86 aa, synthetic construct
  REGION 1..86: note = Endodomain from human IL2R common gamma chain
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 40 - protein, 286 aa, synthetic construct
  REGION 1..286: note = Endodomain from human IL-2R beta
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 41 - protein, 196 aa, synthetic construct
  REGION 1..196: note = Endodomain from human IL-7R alpha
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNEQ

SEQ ID NO: 42 - protein, 54 aa, synthetic construct
  REGION 1..54: note = Endodomain Derived from Human GM-CSFR alpha
KRFLRIQRLFPPVPQIKDKLNDNHEVEDEIIWEEFTPEEGKGYREEVLTVKEIT

SEQ ID NO: 43 - protein, 437 aa, synthetic construct
  REGION 1..437: note = Endodomain from GM-CSFR common beta chain
RFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 44 - protein, 112 aa, synthetic construct
  REGION 1..112: note = mature protein of wild-type TGFbeta2
ALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 45 - protein, 394 aa, synthetic construct
  REGION 1..394: note = TGFbeta monomer sequence
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFRKDLGWKWIHEPKGYNANFCAGACPYRASKSPSCVSQDLEPLTIVYYVGRKPKVEQLSNMIVKSCKCS

SEQ ID NO: 46 - protein, 177 aa, synthetic construct
  REGION 1..177: note = dominant-negative TGFbeta RII (dnTGFbeta RII)
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 47 - protein, 194 aa, synthetic construct
  REGION 1..194: note = MH2 domain of SMAD2
WCSIAYYELNQRVGETFHASQPSLTVDGFTDPSNSERFCLGLLSNVNRNATVEMTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSVRCSSMS

SEQ ID NO: 48 - protein, 194 aa, synthetic construct
  REGION 1..194: note = MH2 domain of SMAD3
WCSISYYELNQRVGETFHASQPSMTVDGFTDPSNSERFCLGLLSNVNRNAAVELTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSIRCSSVS

SEQ ID NO: 49 - protein, 230 aa, synthetic construct
  REGION 1..230: note = MH2 domain of SMAD4
WCSIAYFEMDVQVGETFKVPSSCPIVTVDGYVDPSGGDRFCLGQLSNVHRTEAIERARLHIGKGVQLECKGEGDVWVRCLSDHAVFVQSYYLDREAGRAPGDAVHKIYPSAYIKVFDLRQCHRQMQQQAATAQAAAAAQAAAVAGNIPGPGSVGGIAPAISLSAAAGIGVDDLRRLCILRMSFVKGWGPDYPRQSIKETPCWIEIHLHRALQLLDEVLHTMPIADPQPLD

SEQ ID NO: 50 - protein, 380 aa, synthetic construct
  REGION 1..380: note = chimeric dnSMAD comprising a dnSMAD2 polypeptide and a dnSMAD3 polypeptide
WCSIAYYELNQRVGETFHASQPSLTVDGFTDPSNSERFCLGLLSNVNRNATVEMTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMLEYSGGGSGGGSLEWCSISYYELNQRVGETFHASQPSMTVDGFTDPSNSERFCLGLLSNVNRNAAVELTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQM

SEQ ID NO: 51 - protein, 271 aa, synthetic construct
  REGION 1..271: note = chimeric TNF receptor HVEM-41BB
MEPPGDWGPPPWRSTPRTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVIISFFLALTSTALLFLLFFLTLRFSVVKRGRKKLLYIFKQPFMRPVQTTQEEDGCSCRFPEEEEGGCEL

SEQ ID NO: 52 - protein, 260 aa, synthetic construct
  REGION 1..260: note = chimeric TNF receptor CD27-41BB
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRIISFFLALTSTALLFLLFFLTLRFSVVKRGRKKLLYIFKQPFMRPVQTTQEEDGCSCRFPEEEEGGCEL

SEQ ID NO: 53 - protein, 281 aa, synthetic construct
  REGION 1..281: note = chimeric TNF receptor RANK-41BB
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPIISFFLALTSTALLFLLFFLTLRFSVVKRGRKKLLYIFKQPFMRPVQTTQEEDGCSCRFPEEEEGGCEL

SEQ ID NO: 54 - protein, 149 aa, synthetic construct
  REGION 1..149: note = chimeric TNF receptor Fn14-41BB
MARGSLRRLLRLLVLGLWLALLRSVAGEQAPGTAPCSRGSSWSADLDKCMDCASCRARPHSDFCLGCAAAPPAPFRLLWPIISFFLALTSTALLFLLFFLTLRFSVVKRGRKKLLYIFKQPFMRPVQTTQEEDGCSCRFPEEEEGGCEL

SEQ ID NO: 55 - protein, 261 aa, synthetic construct
  REGION 1..261: note = TNF receptor ligand CD40L
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 56 - protein, 183 aa, synthetic construct
  REGION 1..183: note = TNF receptor ligand OX40L
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 57 - protein, 254 aa, synthetic construct
  REGION 1..254: note = TNF receptor ligand 41BBL
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 58 - skipped

SEQ ID NO: 59 - protein, 7 aa, synthetic construct
  REGION 1..7: note = consensus Tobacco Etch Virus (TEV) cleavage site
ENLYFQS

SEQ ID NO: 60-70 - skipped (11 entries)

SEQ ID NO: 71 - protein, 5 aa, synthetic construct
  REGION 1..5: note = VH CDR, CDR1
SYNIH

SEQ ID NO: 72 - protein, 16 aa, synthetic construct
  REGION 1..16: note = VH CDR, CDR2
VIWAGGSTNYNSALMS

SEQ ID NO: 73 - protein, 10 aa, synthetic construct
  REGION 1..10: note = VH CDR, CDR3
RSDDYSWFAY

SEQ ID NO: 74 - protein, 12 aa, synthetic construct
  REGION 1..12: note = VL CDR, CDR1
RASSSVSSSYLH

SEQ ID NO: 75 - protein, 7 aa, synthetic construct
  REGION 1..7: note = VL CDR, CDR2
STSNLAS

SEQ ID NO: 76 - protein, 9 aa, synthetic construct
  REGION 1..9: note = VL CDR, CDR3
QQYSGYPIT

SEQ ID NO: 77 - protein, 118 aa, synthetic construct
  REGION 1..118: note = humanised KM666 VH sequence
QVQLQESGPGLVKPSQTLSITCTVSGFSLASYNIHWVRQPPGKGLEWLGVIWAGGSTNYNSALMSRLTISKDNSKNQVFLKMSSLTAADTAVYYCAKRSDDYSWFAYWGQGTLVTVSS

SEQ ID NO: 78 - protein, 108 aa, synthetic construct
  REGION 1..108: note = humanised KM666 VL sequence
ENQMTQSPSSLSASVGDRVTMTCRASSSVSSSYLHWYQQKSGKAPKVWIYSTSNLASGVPSRFSGSGSGTDYTLTISSLQPEDFATYYCQQYSGYPITFGQGTKVEIK

SEQ ID NO: 79 - protein, 136 aa, synthetic construct
  REGION 1..136: note = sort/suicide gene RQR8
CPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 80 - protein, 517 aa, synthetic construct
  REGION 1..517: note = suicide gene Rapcasp9
MASRILWHEMWHEGLEEASRLYFGERNVKGMFEVLEPLHAMMERGPQTLKETSFNQAYGRDLMEAQEWCRKYMKSGNVKDLLQAWDLYYHVFRRISKLEYSGGGSLEGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGGSGGGGSGGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 81 - protein, 520 aa, synthetic construct
  REGION 1..520: note = a flexi-IL-12 sequence
MWIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPLATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDSKMYQVEFKTMNAKLLMDPKRQIFLDQNMLAVIDELMQALNFNSETVPQKSSLEEPDFYKTKIKLCILLHAFRIRAVTIDRVMSYLNAS